(19) United States
(12) Patent Application Publication
(10) Pub. No.: US 2017/0321285 A1
(43) Pub. Date: Nov. 9, 2017

(54) NLRC5 AS A BIOMARKER FOR CANCER PATIENTS AND A TARGET FOR CANCER THERAPY

(71) Applicant: THE TEXAS A&M UNIVERSITY SYSTEM, COLLEGE STATION, TX (US)

(72) Inventor: KOICHI KOBAYASHI, BRYAN, TX (US)

(21) Appl. No.: 15/585,761

(22) Filed: May 3, 2017

Related U.S. Application Data
(60) Provisional application No. 62/331,121, filed on May 3, 2016.

(51) Int. Cl. C12Q 1/68; C07K 16/28

(52) U.S. Cl. CPC C12Q 1/6886; C07K 16/2818; C12Q 2600/156; C12Q 2600/154; C12Q 2600/106; C07K 2317/21; C12Q 2600/158; C12Q 2600/112

(57) ABSTRACT

The invention pertains to biomarkers for identifying a cancer that is likely or not likely to evade the immune system of a subject, thus, is likely or not likely to show better prognosis (prognostic biomarker) and/or better responses to cancer therapies (predictive biomarker). The invention provides a method of identifying a subject as having a cancer that is likely to evade the immune system of the subject based on one or more of the following biomarkers in the cancer cells of the subject: a) reduced amount of NLRC5 mRNA or protein; b) reduced activity of NLRC5 protein; c) a mutation that reduces the activity of NLRC5 protein; d) increased methylation of nlrc5 or a portion thereof; and e) reduced copy number of nlrc5. These variables are useful to predict both patient survival (prognostic biomarker) and patient responses to immunotherapies (predictive biomarker). Furthermore, this invention provides a method of identifying a subject as having a cancer that is likely to evade the immune system of the subject with greater prediction power by utilizing multiple variables, in addition to above a)-e) variables, including neoantigen load, mutation number, or expression of genes involved in immune responses, including but not limited to CTLA4, PD1, PD-L1 and PD-L2. The invention also pertains to a method of treating a cancer likely to evade the immune system of the subject by administering an immunotherapy and a therapy designed to activate the MHC class I antigen presentation pathway by activating the expression and/or activity of NLRC5 protein.

### CROSS-REFERENCE TO RELATED APPLICATION

This application claims the benefit of U.S. Provisional Application Ser. No. 62/331,121, filed May 3, 2016, the disclosure of which is hereby incorporated by reference in its entirety, including all figures, tables and amino acid or nucleic acid sequences.

This invention was made with government support under R01DK074738 awarded by National Institutes of Health. The government has certain rights in the invention.

The Sequence Listing for this application is labeled “Seq-List.txt” which was created on Apr. 14, 2017 and is 46 KB. The entire content of the sequence listing is incorporated herein by reference in its entirety.

### BACKGROUND OF THE INVENTION

Cancer immunotherapy, for example, checkpoint blockade using antibodies against cytotoxic T-lymphocyte-associated protein (CTLA4), programmed cell death protein 1 (PD-1) or programmed death ligand 1 (PD-L1), has emerged as a promising cancer treatment. However, its effectiveness is negligible if cancer cells evade anti-tumor immune responses. Indeed, only a fraction of patients with specific cancer types respond to current immunotherapies. Therefore, uncovering the molecular mechanism by which cancer cells escape from the host immune system would facilitate therapeutic strategies with better efficacy for a broader range of cancer types. Loss of MHC class I found in cancer cells at high frequency has been considered as an immune evasion mechanism. While many molecular mechanisms for loss of MHC class I are reported, none of these mechanisms explain the cancer immune evasion phenotype in a broad range of malignancies.

### BRIEF SUMMARY OF THE INVENTION

The invention describes a molecular mechanism by which a cancer cell induces the loss of MHC class I and related pathways of antigen presentation. The invention also provides biomarkers for identifying a subject as having a cancer that is likely or not likely to evade the immune system of the subject, thus useful for predicting patient survival (prognostic biomarker) and for predicting responses to cancer treatment (predictive biomarker). The biomarkers presented herein include the amount of an NLR caspase recruitment domain (CARD) containing 5 (NLRC5) mRNA, the amount of NLRC5 protein, the activity of NLRC5 protein, the level of methylation of nlrc5 or a portion thereof, a mutation in nlrc5, and the copy number of nlrc5. Accordingly, a subject is determined for the prognosis and likeliness to respond to cancer therapy based on the following biomarkers in a sample of cancer cells obtained from the subject:

a) reduced amount of NLRC5 mRNA or NLRC5 protein,

b) reduced activity of NLRC5 protein,

c) a mutation that reduces the activity of NLRC5 protein,

d) increased methylation of nlrc5 or a portion thereof, and

e) reduced copy number of nlrc5.

The invention includes use of these status of NLRC5 as a prognostic and predictive biomarker, by itself or in combination with other variables, including, but not limited to neoantigen load, mutation number, and expression of genes involved in immune responses, including but not limited to CTLA4, PD1, PD-L1 and PD-L2.

The invention also provides kits and reagents to conduct the assays to quantify the biomarkers described herein.

Further, the invention provides methods of treating a subject having cancer. In one embodiment, a methods of treating a subject having a cancer that is likely to evade the immune system of the subject comprises administering a first therapy and a second therapy to the subject, wherein the first therapy is an immunotherapy and the second therapy is designed to reduce the ability of the cancer cells to evade the immune system of the subject. In one embodiment, the second therapy comprises one or more agents that increase the expression of NLRC5 mRNA or NLRC5 protein, increase the activity of NLRC5 protein or induce demethylation of the genomic DNA, particularly of nlrc5 or a portion thereof.

### BRIEF DESCRIPTION OF THE DRAWINGS

The patent or application file contains at least one drawing executed in color. Copies of this patent or patent application publication, with color drawing(s), will be provided by the Office upon request and payment of the necessary fee.

FIGS. 1A-1F. Expression of nlrc5 and MEW class I genes are positively correlated. FIG. 1A. Scatter plots for the expression of nlrc5 (x-axis; log₁₀ values in transcripts per million; TPM) and hla-b (y-axis; log₁₀ values in TPM) in 20 TCGA tumor types (n=7747). FIG. 1B. Spearman rank correlation coefficient between the expression of nlrc5 and hla-b. Sixteen representative tumor types carrying at least 100 samples are shown. FIG. 1C. Scatter plots for the expression of nlrc5 and hla-b in 6 tumor types showing a high correlation coefficient. FIG. 1D. Scatter plots for the expression of nlrc5 and other MHC class I-related genes in melanoma that have the highest correlation coefficient. FIG. 1E. Scatter plots for the expression of nlrc5 and granzyme A (gzma) or perforin (prf1) showing cytolytic activity in 20 TCGA tumor types (n=7749). FIG. 1F. Scatter plots for the expression of nlrc5 and cd8a in 20 TCGA tumor types (n=6277) or cd56 in 19 TCGA tumor types (n=5685). FIGS. 1A-1F. Pairwise correlations were calculated using Spearman's ranked correlation test. r: Spearman rho coefficient.

FIGS. 2A-2J. Preferential DNA-methylation in the nlrc5 promoter in cancer cells is associated with impaired MHC class I-dependent cytotoxic T cell activity. FIG. 2A. Indicated cancer cell lines were treated with 3 μM of 5-azacytidine (5-Aza) for DNA demethylation for the indicated period and the expression of nlrc5 and hla-b were quantified by qPCR. FIG. 2B. Schematic representation of methylation-specific probe on nlrc5 promoter region. The nlrc5 promoter has a CpG island of −578 bp starting at position −278. To examine the methylation status of the nlrc5 promoter, methylation specific probe (cg16411857, blue line) on the CpG island was used. The transcription start site is indicated as −1, NFκB binding site at −313, STAT1 binding site GAS at −570 and a TATA box at −612. Scatter plots show the expression of nlrc5 (y-axis; log₁₀ values in TPM) and methylation level of nlrc5 promoter (x-axis; β values) in 18 TCGA tumor types (n=6523). FIG. 2C. Spearman rank correlation coefficient between nlrc5 expression and DNA methylation of nlrc5 promoter in 15 TCGA tumor types that hold at least 100 samples. FIG. 2D. Scatter plots for the expression of various MHC class I-related genes and methylation level of nlrc5 promoter in thyroid cancer that exhibited the highest negative correlation coefficient. FIG. 2E. Scatter plots for the expression of hla-b and methylation level of ciita promoter in 17 TCGA tumor types (n=5667). FIG. 2F. Scatter plots for the expression of granzyme A (gzma) or perforin (prf1) and methylation level of nlrc5 promoter in 18 TCGA tumor types (n=6528). FIG. 2G. Scatter plots for the expression of cd8a in 18 TCGA tumor types (n=6277) or cd56 and methylation level of nlrc5 promoter in 17 TCGA tumor types (n=5685). FIG. 2H. Dot plots for the methylation level of various MHC class I-related genes (y-axis; β values) in thyroid cancer (n=502) and all cancer types (19 TCGA tumor types, n=6547). The horizontal line corresponds to the median. Statistical significance was determined by the Mann-Whitney test. **: p<0.01. FIG. 2I. Spearman rank correlation coefficient between the expression and methylation of indicated MHC class I-related genes in 19 TCGA tumor types. FIG. 2J. Scatter plots for the expression and methylation level of various MHC class I-related genes in 19 TCGA tumor types. FIGS. 2B-2G, 2I and 2J. Pairwise correlations were calculated using Spearman's ranked correlation test. r: Spearman rho coefficient.

FIGS. 3A-3J. Copy number loss and somatic mutations in nlrc5 are associated with reduced MHC class I gene expressions. FIG. 3A. Percentage of cancer patients who carry nlrc5 copy number (CN) loss among 20 TCGA tumor types. Based on GISTIC values, samples were classified into nlrc5 diploid group and CN loss group: GISTIC 0, diploid; −1 and −2, CN loss. FIG. 3B. Percentage of cancer patients who carry copy number (CN) loss of various MHC class I-related genes for 10 TCGA tumor types for which data are available (bladder, breast, colon, head/neck, lung adeno, lung squamous, ovarian, prostate, rectal and uterine cancer, top) and ovarian cancer (bottom). Statistical significance was determined by chi-square test. *: p<0.01, **: p<0.0001. FIG. 3C. Heatmap showing gene expression of nlrc5 and hla-b in nlrc5 diploid group (n=2028) or CN loss group (n=890) for 17 TCGA tumor types in which the nlrc5 promoter is not methylated. FIG. 3D. The box plots showing nlrc5 and MHC class I-related gene expression in the nlrc5 diploid group or CN loss group in breast cancer patients. FIG. 3E. Pie chart representing the percentage distribution of different types of mutations in nlrc5 in various cancer patients (n=7752). FIG. 3F. Mutation rate in 20 TCGA tumor types. FIG. 3G. Representation of nlrc5 indicating 13 mutations found in at least 2 different cancer patients. FIG. 3H. HEK293T cells were co-transfected with either empty control vector or the respective nlrc5 mutant plasmid with hla-b reporter plasmid and hla-b promoter activity was assessed by the dual-luciferase assay and normalized against Renilla firefly activity. Data are representative of two independent experiments performed in duplicate and is plotted as fold induction with respect to the control vector. Error bar: ±sd. FIG. 3I. Scatter plots for the expression of nlrc5 and hla-b genes for the nlrc5 wild-type group (blue circle) and nlrc5 mutant group (black cross) in 20 TCGA tumor types (n=7752). FIG. 3J. Box plots for the expression level of MHC class I-related genes normalized by the expression level of nlrc5 in 20 TCGA tumor types that are either nlrc5 wild-type or nlrc5 mutant. FIGS. 3D-3J. The horizontal line corresponds to the median, the box to the 25th-75th percentile and the lines to the confidence interval (5th-95th percentile). Statistical significance was determined by the Mann-Whitney test. *:p<0.05, **: p<0.01

FIGS. 4A-4G. Higher expression of nlrc5 is correlated with better survival in multiple cancer types. FIG. 4A. 5-year survival rate in high and low nlrc5 expression groups in 20 TCGA tumor types (right). Difference of 5-year survival rate between nlrc5 high low groups is indicated (left). Patients were divided into 4 groups by the nlrc5 expression level and the top quartiles (nlrc5 high) and the bottom quartiles (nlrc5 low) were analyzed. Statistical significance was determined by the chi-square test. *: p<0.05, **: p<0.01. FIG. 4B. Kaplan-Meier survival curves for indicated tumor types between low and high nlrc5 expression groups. FIG. 4C. Kaplan-Meier survival curves of melanoma for low and high expression groups of the indicated NLRC5-dependent MHC class I-related genes. FIG. 4D. Kaplan-Meier survival curves of melanoma for low and high expression groups of the indicated NLRC5-independent MHC class I-related genes. FIG. 4E. Kaplan-Meier survival curves of melanoma for low and high expression groups of the CD8A and indicated markers for cytotoxic CD8⁺ T cell activity. FIG. 4F. Kaplan-Meier survival curves of melanoma for low and high methylation groups of the nlrc5 promoter and the indicated MHC class I-related genes. FIGS. 4B-4F. Statistical significance was determined by the log-rank and Gehan-Breslow-Wilcoxon tests. FIG. 4G. Model of cancer evolution targeting NLRC5 for immune evasion. NLRC5-dependent MHC class I expression is crucial for CD8⁺ T cell-mediated anti-tumor responses and the elimination of cancer cells. Genetic and epigenetic changes, such as mutations, copy number loss or promoter methylation of the nlrc5 occur during the evolution of cancer cells, leading to an impaired MHC class I system. These changes result in the impaired ability to elicit anti-tumor CD8⁺ T cell responses and reduced infiltration in cancer tissues. Cancer cells successful at immune evasion cause efficient tumor development, leading to poor prognosis of cancer-bearing patients. Cancer cells (gray) and CD8⁺ T cells (orange) are shown.

FIGS. 5A-5B. Expression of nlrc5 and MHC Class I genes are positively correlated. FIG. 5A. Scatter plots for the expression of nlrc5 (x-axis; log₁₀ values in TPM) and other MHC class I-related genes (y-axis; log₁₀ values in TPM) in biopsy samples from patients of bladder cancer (top), thyroid cancer (middle) and breast cancer (bottom). FIG. 5B. Scatter plots for the expression of nlrc5 and granzyme A (GZMA, top) or perforin (PRF1, bottom) for 6 indicated TCGA tumor types. (FIGS. 5A and 5B) Pairwise correlations were calculated using Spearman's ranked correlation test. r: Spearman rho coefficient.

FIGS. 6A-6D. Methylation level of nlrc5 promoter and the expression of MHC class I genes are negatively correlated. FIG. 6A. Schematic representation of methylation-specific probe on nlrc5 promoter region. The nlrc5 promoter has a CpG island of −578 bp starting at position −278. To examine the methylation status of the nlrc5 promoter, a methylation specific probe (cg16411857, blue line) on the CpG island was used. The transcription start site is indicated as −1, the NFκB binding site at −313, the STAT1 binding site GAS at −570 and a TATA box at −612. FIG. 6B. Scatter plots for the expression of nlrc5 or hla-b expression (y-axis; log₁₀ values in TPM) and methylation level of nlrc5 promoter (x-axis; β values) in indicated tumor types showing negative correlation coefficient. FIG. 6C. Scatter plots for the expression of various MHC class I-related genes and methylation level of nlrc5 promoter in bladder cancer (top), uterine cancer (middle) and melanoma (bottom). FIG. 6D. Dot plots for the methylation level of the nlrc5 promoter (x-axis; β values) in 15 TCGA tumor types that hold at least 100 samples. The vertical line corresponds to the median. (FIGS. 6B and 6C) Pairwise correlations were calculated using Spearman's ranked correlation test. r: Spearman rho coefficient.

FIGS. 7A-7C. Copy number loss in nlrc5 is associated with reduced MHC class I gene expression. FIG. 7A. The box plots showing nlrc5 and MHC class I-related gene expression in the nlrc5 diploid group or copy number (CN) loss group in 20 TCGA tumor types. FIG. 7B. Reduction rate of nlrc5 expression calculated by using the mean expression of nlrc5 of the CN loss group divided by the mean of the diploid group in 8 TCGA tumor types that have at least 100 samples. FIG. 7C. The box plots showing nlrc5 and MHC class I-related gene expression in the nlrc5 diploid group or CN loss group in melanoma (top left), hepatocellular carcinoma (top right), ovarian cancer (bottom left) and lung adenocarcinoma (bottom right). Based on GISTIC values, samples were classified into the nlrc5 diploid group and CN loss group: GISTIC 0, diploid; −1 and −2, CN loss. (FIGS. 7A and 7C) The horizontal line corresponds to the median, the box to the 25th-75th percentile and the lines to the confidence interval (5th-95th percentile). Statistical significance was determined by the Mann-Whitney test. *: p<0.05, **:p<0.01.

FIG. 8. Somatic mutation in nlrc5. Positional representation of 161 mutations in nlrc5. Black bar; mutation found in one patient. Red bar; mutation found in at least two patients.

FIGS. 9A-9C. Survival curves of melanoma and bladder cancer in high and low expression or methylation groups of the indicated genes. FIG. 9A. Kaplan-Meier survival curves of melanoma for high and low expression groups of the indicated MHC class I-related genes. FIG. 9B. Kaplan-Meier survival curves of melanoma for high and low methylation groups of the indicated MHC class I genes. FIG. 9C. Kaplan-Meier survival curves of bladder cancer for high and low methylation groups of the nlrc5 promoter and the indicated MHC class I-related genes. (FIGS. 9A to 9C) Statistical significance was determined by the log-rank and Gehan-Breslow-Wilcoxon tests.

FIGS. 10A-10C. The expression of NLRC5 and MHC class I associated genes are correlated with response to anti-CTLA4 antibody therapy. The transcript levels of FIG. 10A NLRC5, FIG. 10B HLA-B, B2M, FIG. 10C CD8A, granzyme A (GZMA), perforin (PRF1) and CD56 between patient groups who benefited from anti-CTLA4 antibody therapy (Response, n=14) and who did not (Nonresponse, n=23). Bar represents the median value. P-values calculated using Mann-Whitney U test.

FIGS. 11A-11D. Multivariate analysis with NLRC5 expression and load of mutation or neoantigen provide predictive information for the response to anti-CTLA4 therapy. FIG. 11A: Comparison of mutation and neoantigen load between response (n=13) and non-response (n=22) groups. P-values were calculated using Mann-Whitney U test. FIG. 11B: Scatterplots for NLRC5 expression and mutation or neoantigen load (left panel). 95% confidence ellipses about the centroids were drawn for both response (red circle in right panel) and non-response group (blue circle in right panel). P-values were calculated using Hotelling's Test. FIG. 11C: Response rate to anti-CTLA4 therapy in the four groups stratified with NLRC5 expression and mutation/neoantigen load. Cohort was divided into four groups based on the level of NLRC5 expression and mutation or neoantigen load. The response rate (%) to the therapy among each group was calculated. Patients carrying higher value of the median are defined as high group (H), those carrying lower value of the median are defined as low group (L) in respective variables. Statistical significance between the groups of high NLRC5 expression/high mutation or neoantigen load and low NLRC5 expression/low mutation or neoantigen load were determined by the χ2 test. FIG. 11D: ROC curves for logistic regression models using the respective combination of variables. The curves represent a model with NLRC5 expression (dotted line in both panel), combination of NLRC5 expression and mutation load (solid line in left panel) and combination of NLRC5 expression and neoantigen load (solid line in right panel). The numbers with arrow are showing false positive rate with 100% sensitivity. AUC, area under the curve.

FIGS. 12A-12B. Combination of PDL2 expression with NLRC5 expression and mutation or neoantigen load are sensitive predictors for responses to anti-CTLA4 therapy. FIG. 12A: Scatterplots for NLRC5 and PDL2 expression with mutation load (left panel) or neoantigen load (right panel) for response (n=13) and nonresponse (n=22) groups. FIG. 12B: ROC curves for logistic regression models using the respective combination of variables. The curves represent a model with PDL2 expression (dotted line in both panel), combination of PDL2 expression, NLRC5 expression and mutation load (solid line in left panel) and combination of PDL2 expression, NLRC5 expression and neoantigen load (solid line in right panel). The numbers with arrow are showing false positive rate with 100% sensitivity. AUC, area under the curve.

FIGS. 13A-13C. Combination of NLRC5 expression and load of mutation or neoantigen provide prognostic information for the response to anti-CTLA4 therapy. FIG. 13A: Overall survival of patients with high and low of mutation load (Left), NLRC5 gene expression (Middle), and NLRC5 methylation (Right). Patients in the TCGA melanoma cohort (n=328) were divided into top 50% and bottom 50% hazard (group of high and low, respectively) using Cox regression model. FIG. 13B: Overall survival of patients with varying levels of two factors, NLRC5 expression and mutation load (Left) and NLRC5 methylation and mutation load (Right). Patients were stratified by two factors (NLRC5 expression/NLRC5 methylation and mutation load) in a similar fashion with (A), yielding four groups (high NLRC5 expression/NLRC5 methylation and high mutation load, likewise, high and low, low and high, low and low). Cox regression model was used to analyze the survival in respective groups. FIG. 13C (Top): Five-year survival rate in the indicated groups. Statistical significance was determined by the χ2 test: *P<0.05; **P<0.01. (Bottom) Maximum difference of 5-year survival rate in respective groups. Difference in the 5-year survival rate was showing in groups with high and low mutation load, NLRC5 expression or NLRC5 methylation were indicating. For combination of NLRC5 expression and mutation load, difference between higher in both NLRC5 expression and mutation load and lower in both was showing. For combination of NLRC5 methylation and mutation load, difference between the combination of lower NLRC5 expression/higher mutation load and higher NLRC5 methylation/lower mutation load was exhibiting.

FIGS. 14A-14B. MHC class I associated gene expressions are correlated with response to anti-CTAL4 therapy. FIG. 14A: A heat map for MEW Class I related genes generated using GSEA comparing response (n=13) and nonresponse (n=22) groups. Each rectangle represents a single patient. Significant upregulation of the genes was found in the response group with a p-value of 0.0596. FIG. 14B: Scatterplots for NLRC5 expression and indicated gene expressions for response (n=14) and nonresponse group (n=23). Pearson's correlation coefficient (r) and associated p-value were indicated.

FIG. 15. Mutation load and neoantigen load are highly correlated in anti-CTLA4 treated melanoma. Scatterplots for mutation load and neoantigen load in response (n=14) and nonresponse (n=23) groups. Pearson's correlation coefficient (r) and associated p-value were indicating.

FIG. 16. Multicollinearity between variables. Scatterplot matrix to detect multicollinearity between variables, including expression of NLRC5, CTLA-4, PD-1, PD-L1 and PD-L2, mutation load and neoantigen load, considered for logistic regression model. Upper panels depict the Pearson's correlation coefficient (r) with associated p-value.

### BRIEF DESCRIPTION OF THE SEQUENCES

SEQ ID NO: 1: Sequence of protein coding region of nlrc5.

SEQ ID NO: 2: Sequence of NLRC5 protein.

SEQ ID NO: 3: Sequence of NLRC5 mRNA.

SEQ ID NOs: 4-5: Sequences of nlrc5 promoter.

SEQ ID NO: 6: Sequence of a portion of nlrc5 promoter.

(CGGAGCTCAGGTGGGTGGGGACCCTGGGCCAAGACCCTGTCTCAGTG
CCT)

SEQ ID NO: 7-34: Sequences of the primers used for construction of selected nlrc5 mutant expression vectors as indicated in Table 3.

### DETAILED DISCLOSURE OF THE INVENTION

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”. The transitional terms/phrases (and any grammatical variations thereof) “comprising”, “comprises”, “comprise”, “consisting essentially of”, “consists essentially of”, “consisting” and “consists” can be used interchangeably. The term “and/or” is used in the context of items within the list connected by the term to mean each item singly or each item together (e.g., “1 and/or 2” means 1 alone, or 2 alone, or 1 and 2 together, or any combination of items recited within the listing). The phrase “one or more of the following biomarkers” means one of the biomarkers within the list (a single biomarker) or any combination of the listed biomarkers.

The term “about” or “approximately” means within an acceptable error range for the particular value as determined by one of ordinary skill in the art, which will depend in part on how the value is measured or determined, i.e., the limitations of the measurement system. Alternatively, “about” can mean a range of 0 to 10% of a given value.

The pharmaceutical agents described in the invention can be formulated in a pharmaceutical composition. A pharmaceutical composition comprises the active agent and a pharmaceutically acceptable carrier or excipient. “Pharmaceutically acceptable carrier or excipient” includes any and all solvents, dispersion media, coatings, antibacterial and antifungal agents, isotonic and absorption delaying agents and the like. The use of such media and agents for pharmaceutically active substances is well-known in the art. Except insofar as any conventional media or agent is incompatible with the antigen in the vaccine, its use in the vaccine compositions of the invention is contemplated.

“Treatment”, “treating”, “palliating” and “ameliorating” (and grammatical variants of these terms), as used herein, are used interchangeably. These terms refer to an approach for obtaining beneficial or desired results including but not limited to therapeutic benefit. A therapeutic benefit is achieved with the eradication or amelioration of one or more of the physiological symptoms associated with the underlying cancer such that an improvement is observed in the patient, notwithstanding that the patient may still be afflicted with the cancer.

As used herein, the term “cancer” refers to the presence of cells possessing abnormal growth characteristics, such as uncontrolled proliferation, immortality, metastatic potential, rapid growth and proliferation rate, perturbed oncogenic signaling, and certain characteristic morphological features. This includes but is not limited to the growth of: (1) benign or malignant cells (e.g., tumor cells) that correlates with overexpression of a serine/threonine kinase, or (2) benign or malignant cells (e.g., tumor cells) that correlates with abnormally high levels of serine/threonine kinase activity or lipid kinase activity. Non-limiting serine/threonine kinases implicated in cancer include but are not limited to PI-3K, mTOR, and AKT. Exemplary lipid kinases include but are not limited to PI3 kinases such as PBKα, PBKβ, PBKδ, and PBKγ.

The term “effective amount” or “therapeutically effective amount” refers to that amount of an inhibitor described herein that is sufficient to effect the intended application, including but not limited to disease treatment. The therapeutically effective amount may vary depending on the intended application (in vitro or in vivo) or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of proliferation or downregulation of activity of a target protein. The specific dose will vary depending on the particular compounds chosen, the dosing regimen to be followed, whether it is administered in combination with other compounds, timing of administration, the tissue to which it is administered, and the physical delivery system in which it is carried.

“Subject” refers to an animal, such as a mammal, for example a human. The methods described herein can be useful in both humans and non-human animals. In some embodiments, the subject is a mammal (such as an animal model of disease), and in some embodiments, the subject is a human. The terms “subject” and “patient” can be used interchangeably.

The terms “antagonist” and “inhibitor” may be used interchangeably, and they refer to a compound having the ability to inhibit a biological function of a target protein, whether by inhibiting the activity or expression of the target protein. Accordingly, the terms “antagonist” and “inhibitor” are defined in the context of the biological role of the target protein. The terms “agonists” and “activators” and their synonyms may be used interchangeably, and they refer to a compound having the ability to activate a biological function of a target protein, whether by increasing the activity or expression of the target protein. Accordingly, the terms “agonist” and “activator” are defined in the context of the biological role of the target protein.

Cancers suitable for treatment according to the disclosed methods include, but are not limited to: Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-related cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone cancer, Bone tumor, Breast cancer, Brenner tumor, Bronchial tumor, Bronchioloalveolar carcinoma, Brown tumor, Burkitt's lymphoma, Cancer of unknown primary site, Carcinoid tumor, Carcinoma, Carcinoma in situ, Carcinoma of the penis, Carcinoma of unknown primary site, Carcinosarcoma, Castleman disease, Central nervous system embryonal tumor, Cerebellar astrocytoma, Cerebral astrocytoma, Cervical cancer, Cholangiocarcinoma, Chondroma, Chondrosarcoma, Chordoma, Choriocarcinoma, Choroid plexus papilloma, Chronic lymphocytic leukemia, Chronic monocytic leukemia, Chronic myelogenous leukemia, Chronic myeloproliferative disorder, Chronic neutrophilic leukemia, Clear-cell tumor, Colon cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial uterine cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing family of tumors, Ewing sarcoma, Extracranial germ cell tumor, Extragonadal germ cell tumor, Extrahepatic bile duct cancer, Extramammary Paget's disease, Fallopian tube cancer, Fetus in fetu, Fibroma, Fibrosarcoma, Follicular lymphoma, Follicular thyroid cancer, Gallbladder cancer, Ganglioglioma, Ganglioneuroma, Gastric cancer, Gastric lymphoma, Gastrointestinal cancer, Gastrointestinal carcinoid tumor, Gastrointestinal stromal tumor, Germ cell tumor, Germinoma, Gestational choriocarcinoma, Gestational trophoblastic tumor, Giant cell tumor of bone, Glioblastoma multiforme, Glioma, Gliomatosis cerebri, Glomus tumor, Glucagonoma, Gonadoblastoma, Granulosa cell tumor, Hairy cell leukemia, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin's lymphoma, Hypopharyngeal cancer, Hypothalamic glioma, Inflammatory breast cancer, Intraocular melanoma, Islet cell carcinoma, Islet cell tumor, Juvenile myelomonocytic leukemia, Kaposi's sarcoma, Kidney cancer, Klatskin tumor, Krukenberg tumor, Laryngeal cancer, Lentigo maligna melanoma, Leukemia, Lip and oral cavity cancer, Liposarcoma, Lung cancer, Luteoma, Lymphangioma, Lymphangiosarcoma, Lymphoepithelioma, Lymphoid leukemia, Lymphoma, Macroglobulinemia, Malignant fibrous histiocytoma, Malignant fibrous histiocytoma of bone, Malignant glioma, Malignant mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloepithelioma, Melanoma, Meningioma, Merkel cell carcinoma, Mesothelioma, Metastatic squamous neck cancer with occult primary, Metastatic urothelial carcinoma, Mixed Mullerian tumor, Monocytic leukemia, Mouth cancer, Mucinous tumor, Multiple endocrine neoplasia syndrome, Multiple myeloma, Mycosis fungoides, Myelodysplasia disease, Myelodysplasia syndromes, Myeloid leukemia, Myeloid sarcoma, Myeloproliferative disease, Myxoma, Nasal cavity cancer, Nasopharyngeal cancer, Nasopharyngeal carcinoma, Neoplasm, Neurinoma, Neuroblastoma, Neurofibroma, Neuroma, Nodular melanoma, Non-Hodgkin's lymphoma, Nonmelanoma skin cancer, Non-small cell lung cancer, Ocular oncology, Oligoastrocytoma, Oligodendroglioma, Oncocytoma, Optic nerve sheath meningioma, Oral cancer, Oropharyngeal cancer, Osteosarcoma, Ovarian cancer, Ovarian epithelial cancer, Ovarian germ cell tumor, Ovarian low malignant potential tumor, Paget's disease of the breast, Pancoast tumor, Pancreatic cancer, Papillary thyroid cancer, Papillomatosis, Paraganglioma, Paranasal sinus cancer, Parathyroid cancer, Penile cancer, Perivascular epithelioid cell tumor, Pharyngeal cancer, Pheochromocytoma, Pineal parenchymal tumor of intermediate differentiation, Pineoblastoma, Pituicytoma, Pituitary adenoma, Pituitary tumor, Plasma cell neoplasm, Pleuropulmonary blastoma, Polyembryoma, precursor T-lymphoblastic lymphoma, Primary central nervous system lymphoma, Primary effusion lymphoma, Primary hepatocellular cancer, Primary liver cancer, Primary peritoneal cancer, Primitive neuroectodermal tumor, Prostate cancer, Pseudomyxoma peritonei, Rectal cancer, Renal cell carcinoma, Respiratory tract carcinoma involving the NUT gene on chromosome 15, Retinoblastoma, Rhabdomyoma, Rhabdomyosarcoma, Richter's transformation, Sacrococcygeal teratoma, Salivary gland cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary syndrome, Signet ring cell carcinoma, Skin cancer, Small blue round cell tumor, Small cell carcinoma, Small cell lung cancer, Small cell lymphoma, Small intestine cancer, Soft tissue sarcoma, Somatostatinoma, Soot wart, Spinal cord tumor, Spinal tumor, Splenic marginal zone lymphoma, Squamous cell carcinoma, Stomach cancer, Superficial spreading melanoma, Supratentorial primitive neuroectodermal tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat cancer, Thymic carcinoma, Thymoma, Thyroid cancer, Transitional cell cancer of renal pelvis and ureter, Transitional cell carcinoma, Urachal cancer, Urethral cancer, Urogenital neoplasm, Uterine sarcoma, Uveal melanoma, Vaginal cancer, Verner-Morrison syndrome, Verrucous carcinoma, Visual pathway glioma, Vulvar cancer, Waldenstrom macroglobulinemia, Warthin's tumor, Wilms' tumor, or any combinations thereof.

In some embodiments, the cancer treated according to the invention is a cancer of the skin, particularly a melanoma; rectum; bladder; cervix; head/neck; thyroid; breast; prostate; uterus; colon; adrenal; hepatocellular; lung, particularly a lung adenoma or lung squamous cancer; or kidney, particularly kidney chromophobe, kidney clear cell or kidney papillary cancer; or a glioma/glioblastoma.

As used herein, the name of a gene is written in lower case and italicized font and the word “gene” may not be spelled out, whereas the name of a protein or an mRNA is written in capital letters and regular font and whether a recitation refers to an mRNA or protein is specified wherever needed. For example, the term “nlrc5” (lower case and italicized) indicates “nlrc5 gene”, whereas the term “NLRC5” indicates NLRC5 protein or mRNA and is specified as mRNA or protein wherever needed.

Cancer cell development occurs under immune surveillance, thus necessitating immune escape for cancer growth. Cancer cells accumulate numerous mutations that constitute potentially immunogenic neo-epitopes. Thus, most tumors concurrently need to employ mechanisms that enable evasion from immune surveillance for successful cancer growth and progression. Cancer cells use multiple strategies of immune evasion, including increased resistance to cytotoxic T cell killing, induction of anergy in activated T cells, elimination of effector T cells, recruitment of regulatory immune cell subsets and reduced recognition of tumor-associated antigens by effector T-cells.

Impaired MEW class I-mediated antigen presentation is a major immune evasion mechanism in cancer, with MHC class I loss reported in cervical cancer (92%), penile cancer (80%), breast cancer (71%), non-small cell lung cancer (64%) and esophageal squamous cell carcinoma (67%), among others. Although a number of mechanisms have been described for HLA loss, including the loss of heterozygosity, HLA gene methylation, nonsense/missense mutations, and loss of tap1/2 or b2m, the dominant underlying molecular mechanism seems to reside at the transcriptional level. Transcriptional regulation of MHC class I genes remained largely undefined until the recent discovery of MHC class I transcriptional activator (CITA), known as NLRC5. NLRC5 is an IFN-γ-inducible nuclear protein that specifically associates with and activates promoters of MHC class I genes by generating a CITA enhanceosome complex with other transcription factors. A striking feature of NLRC5 is that it does not induce only MHC class I genes but also activates other critical genes involved in the MHC class I antigen presentation pathway, including the immunoproteasome component lmp2 (psmb9), peptide transporter tap1 and β2-microglobulin (b2m), thus regulating the entire MHC class I antigen presentation machinery. Nlrc5-deficient mice exhibit impaired constitutive and inducible expression of MHC class I genes. In addition, Nlrc5-deficient cells display an impaired ability to elicit CD8⁺ T cell activation, as evidenced by impaired IFN-γ production and diminished cytolytic activity.

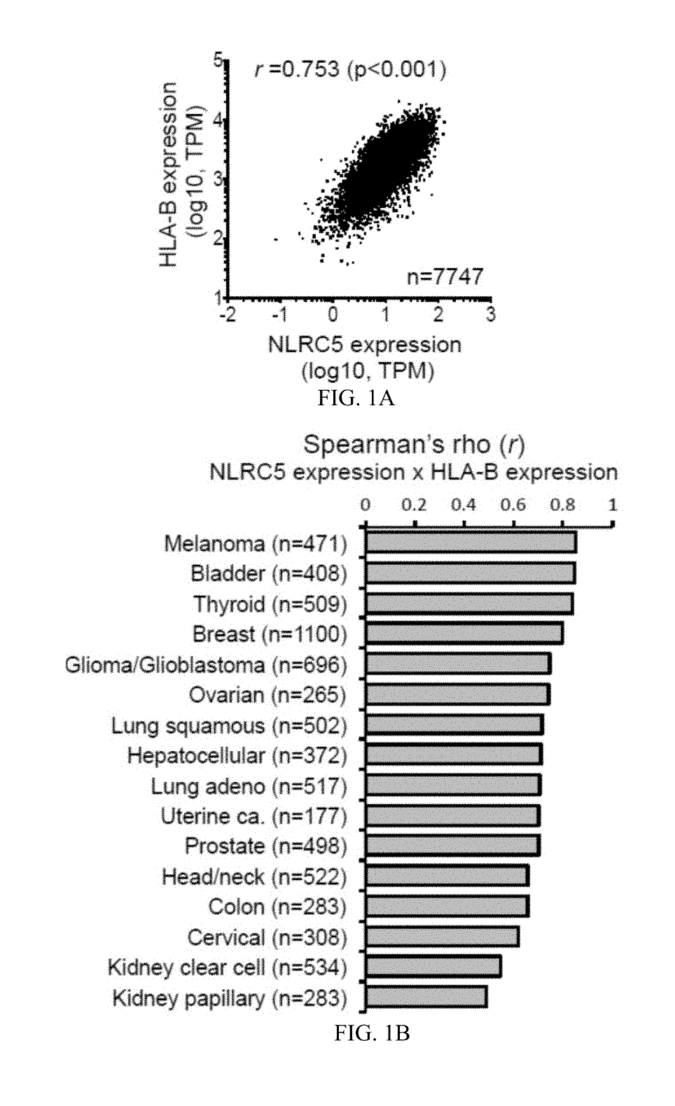

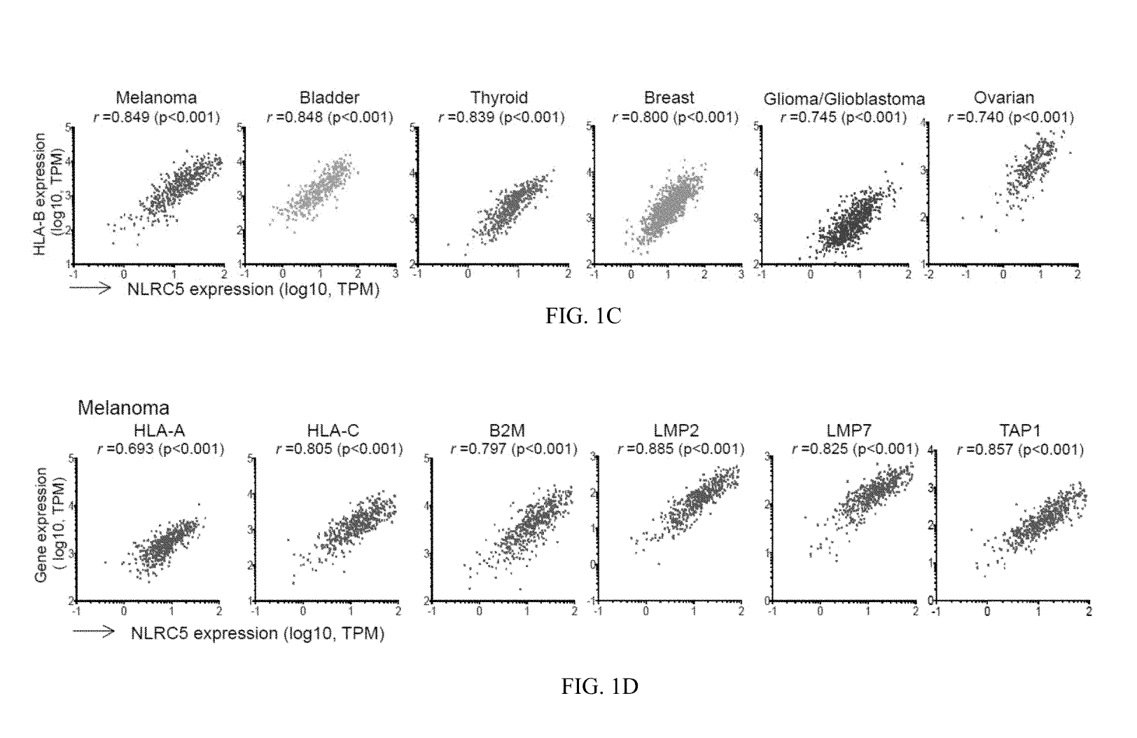

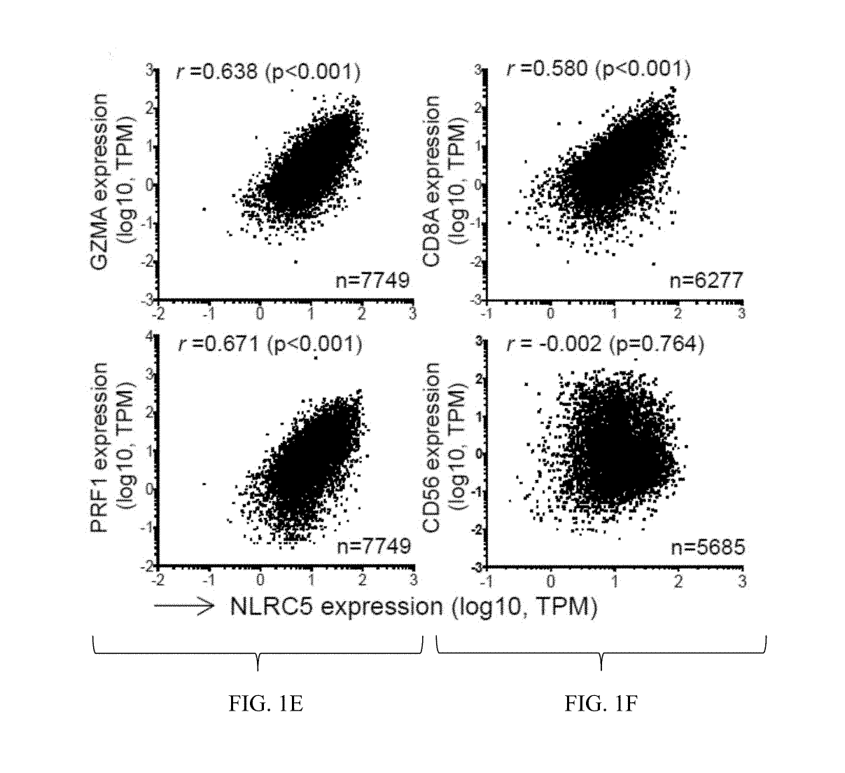

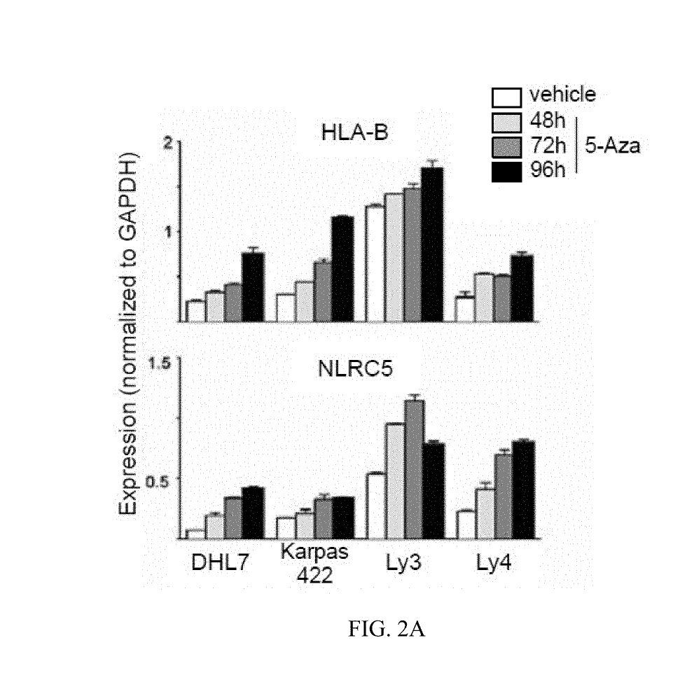

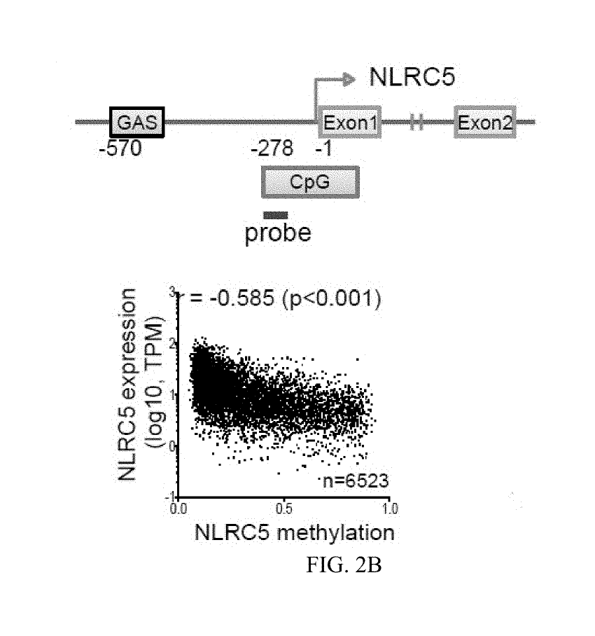

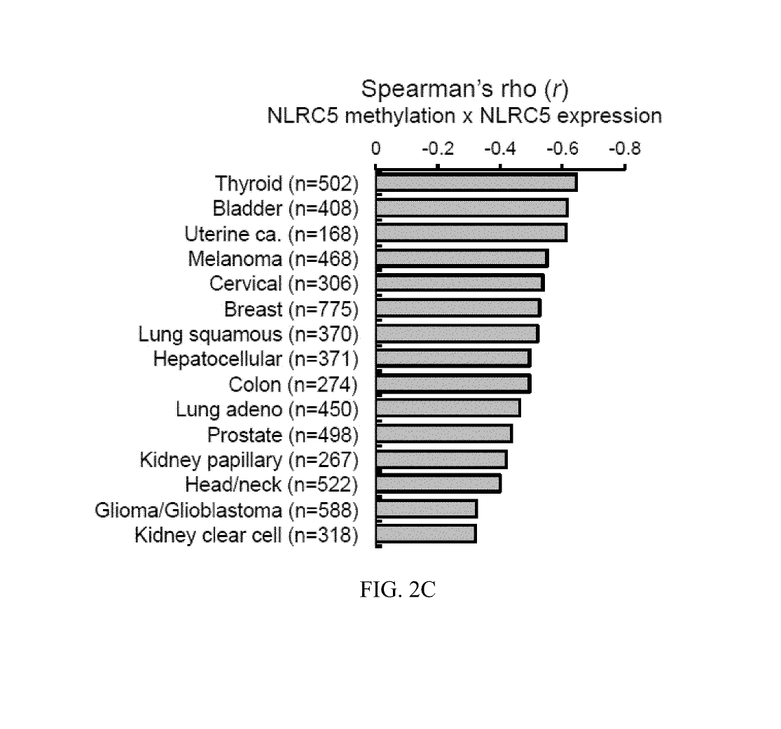

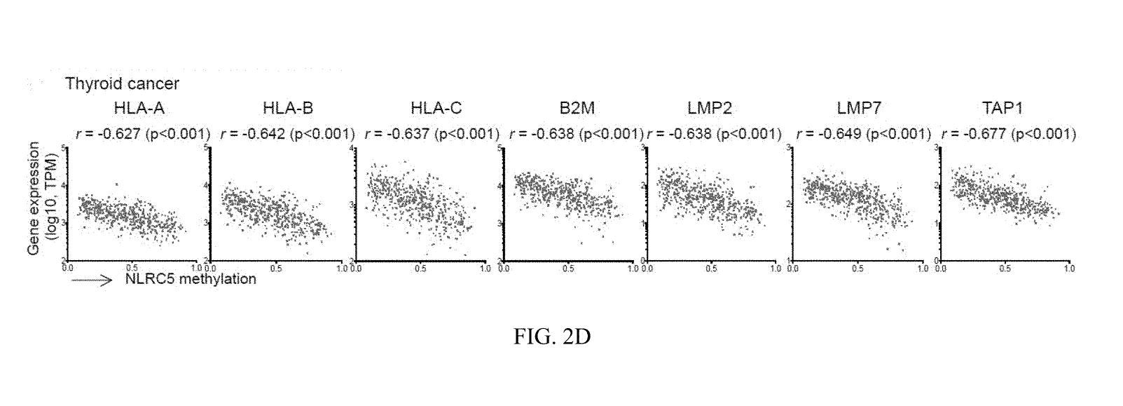

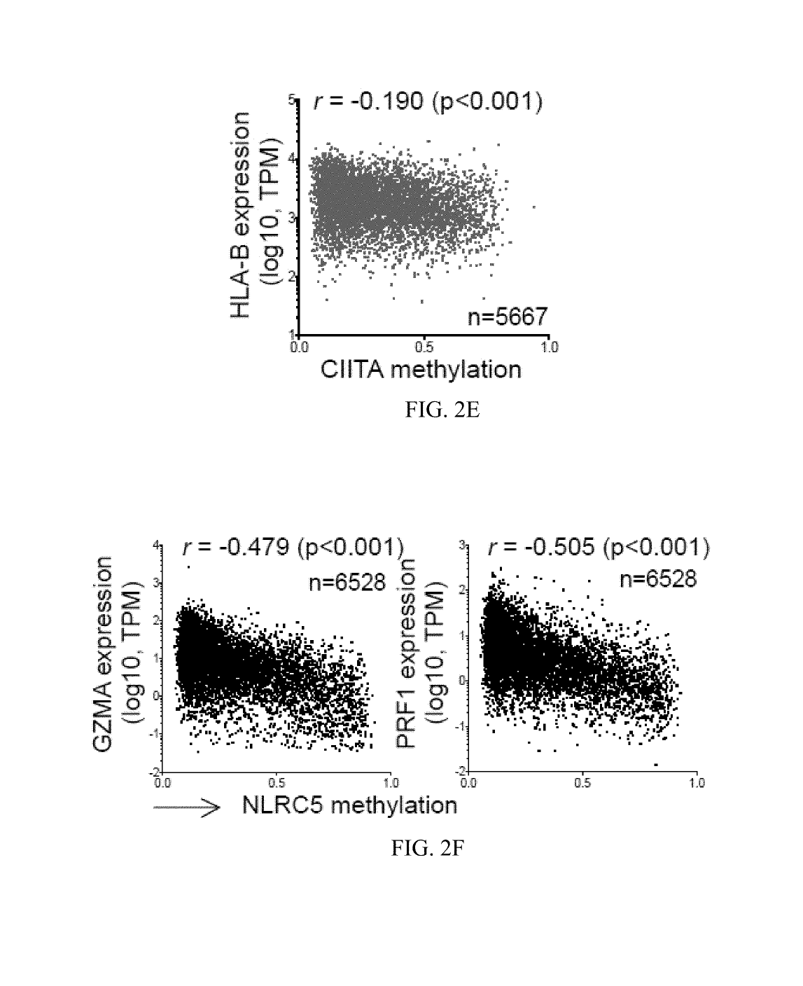

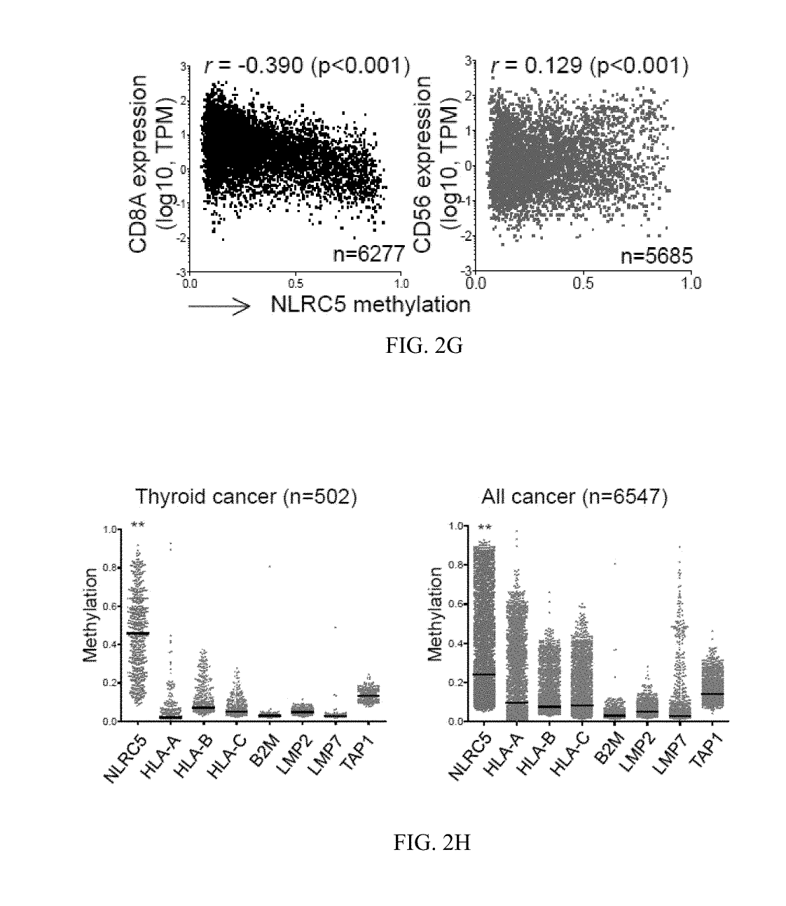

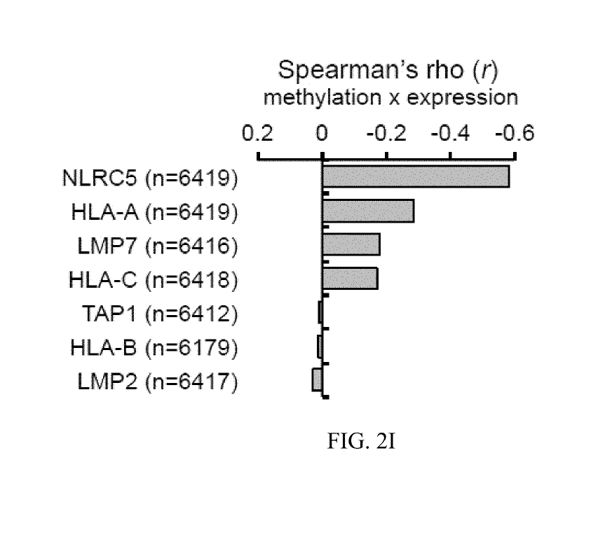

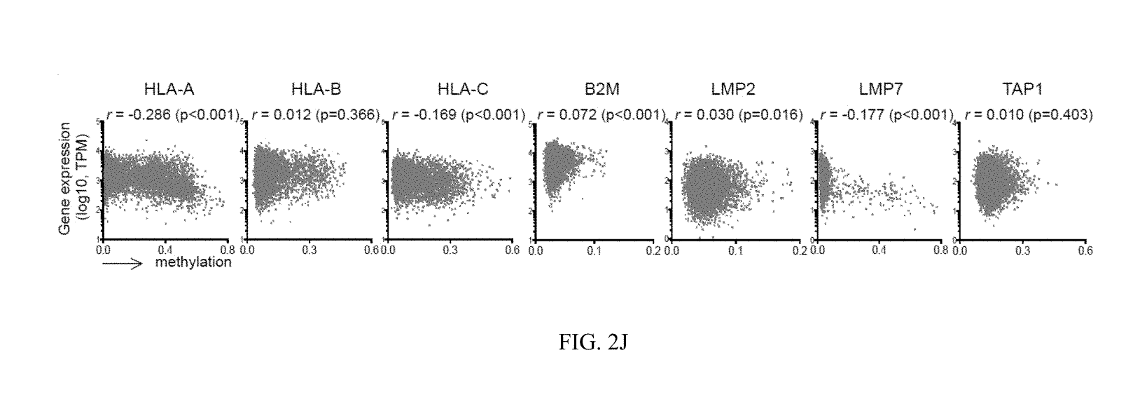

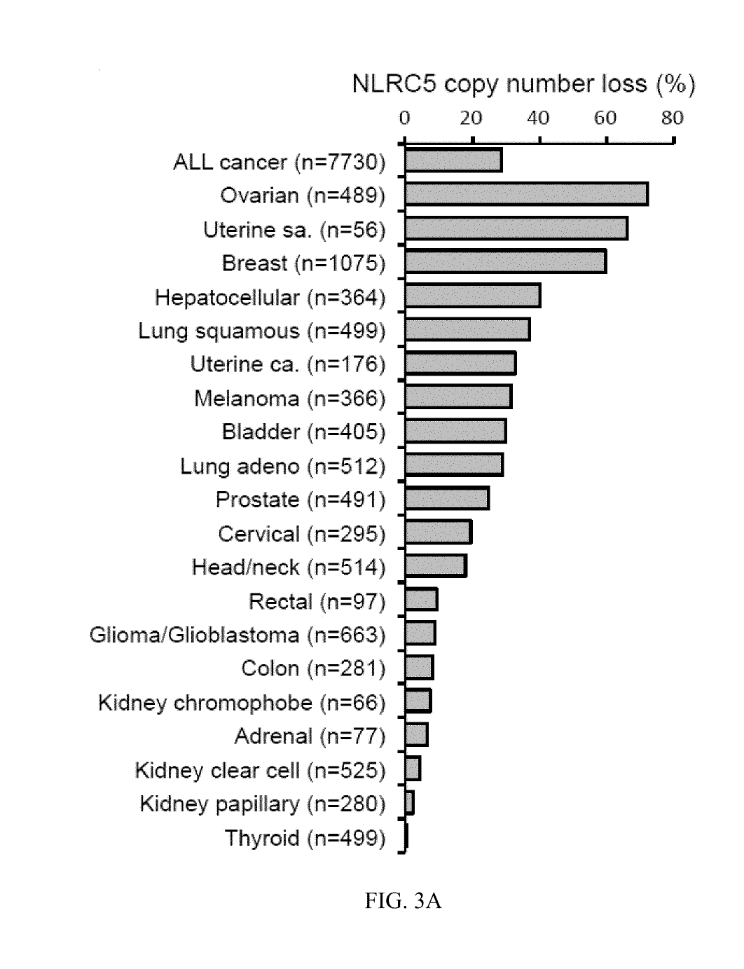

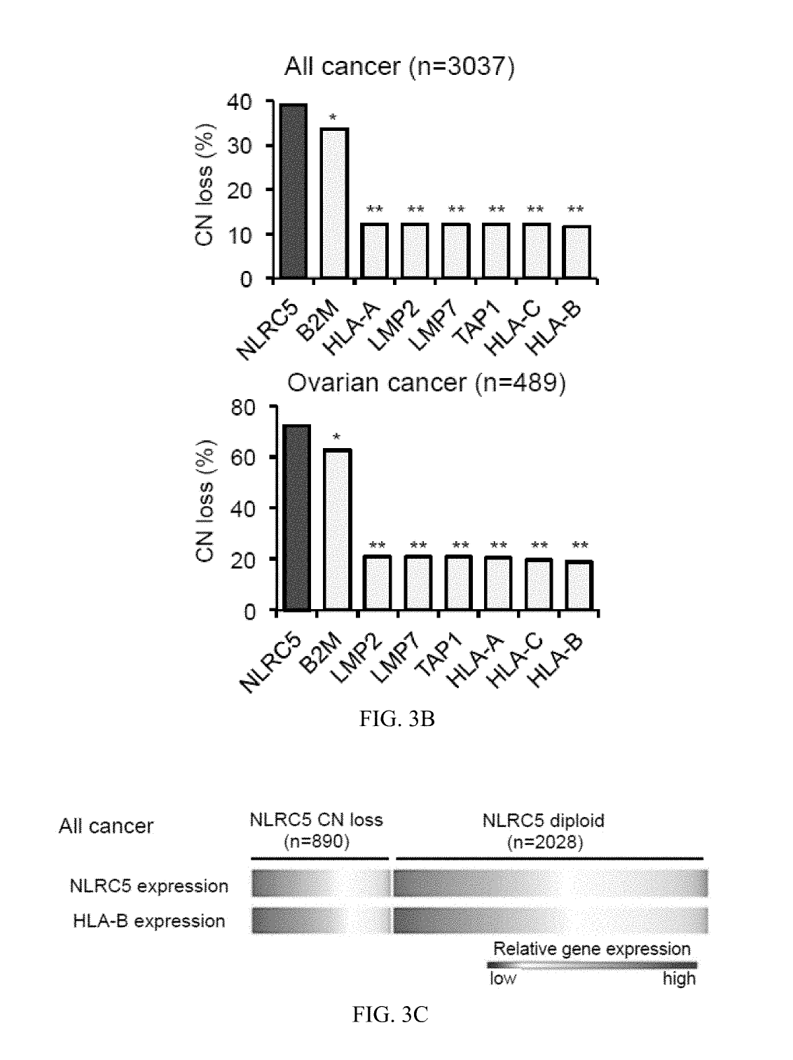

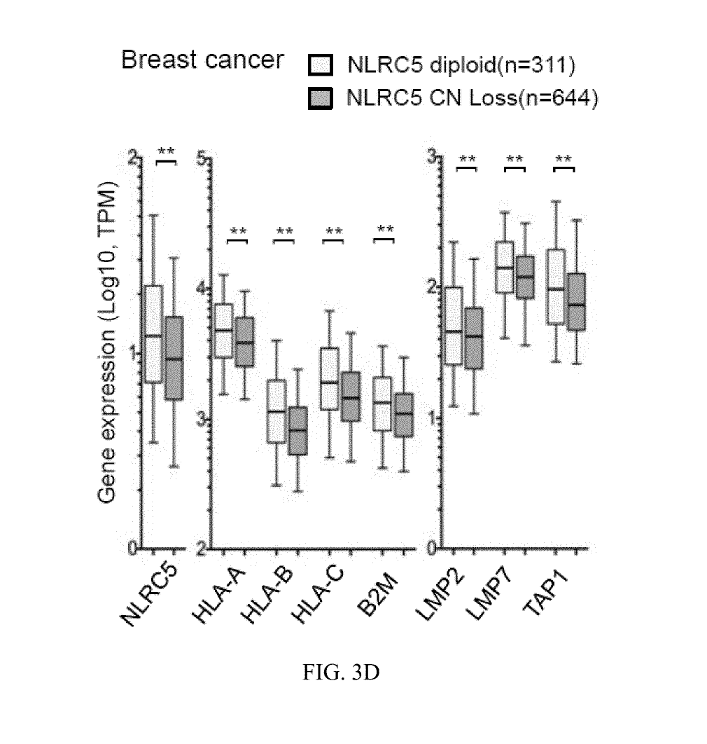

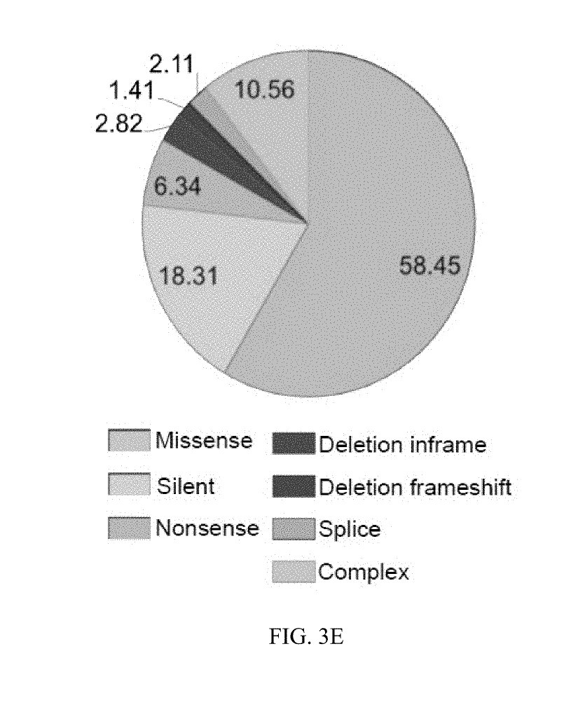

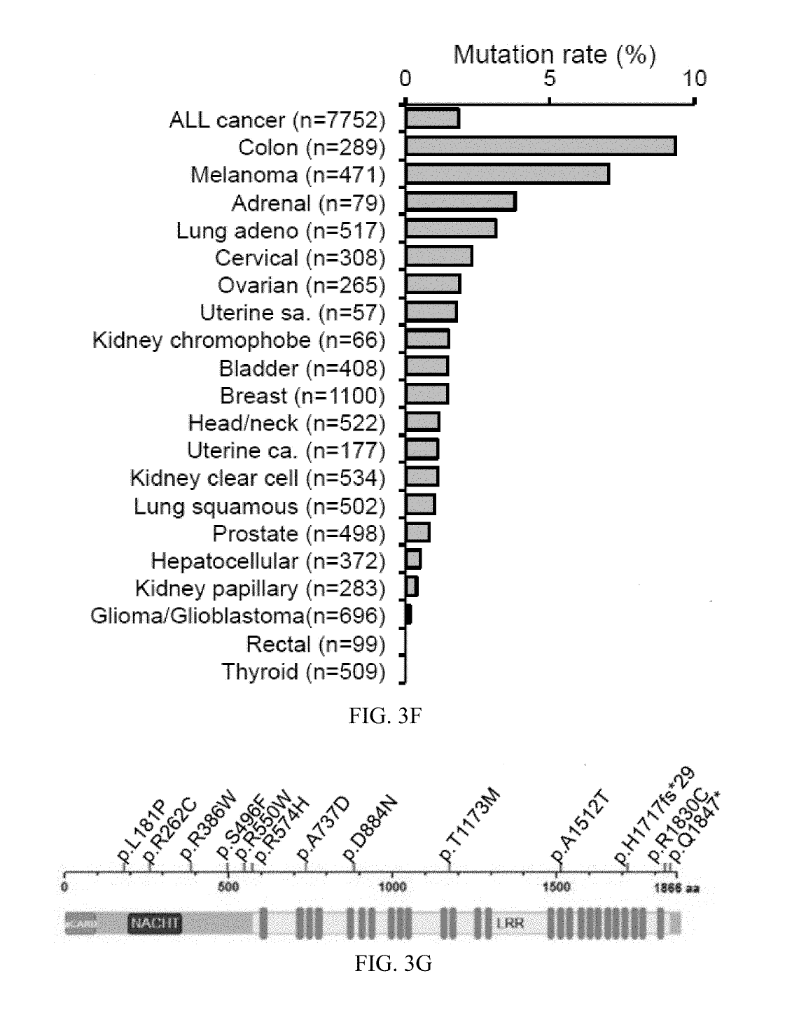

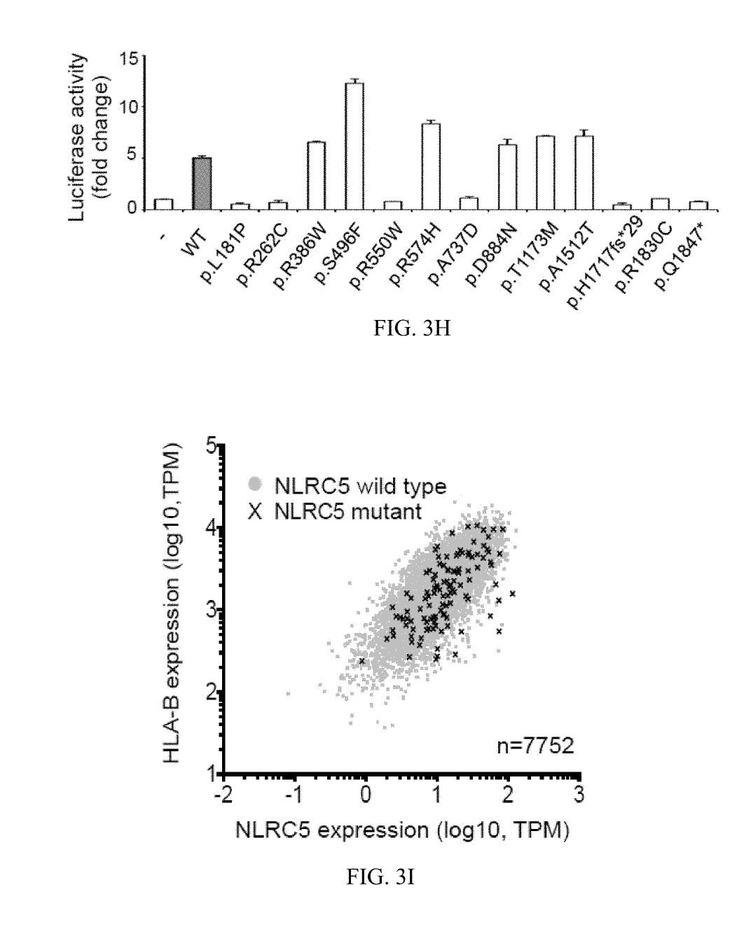

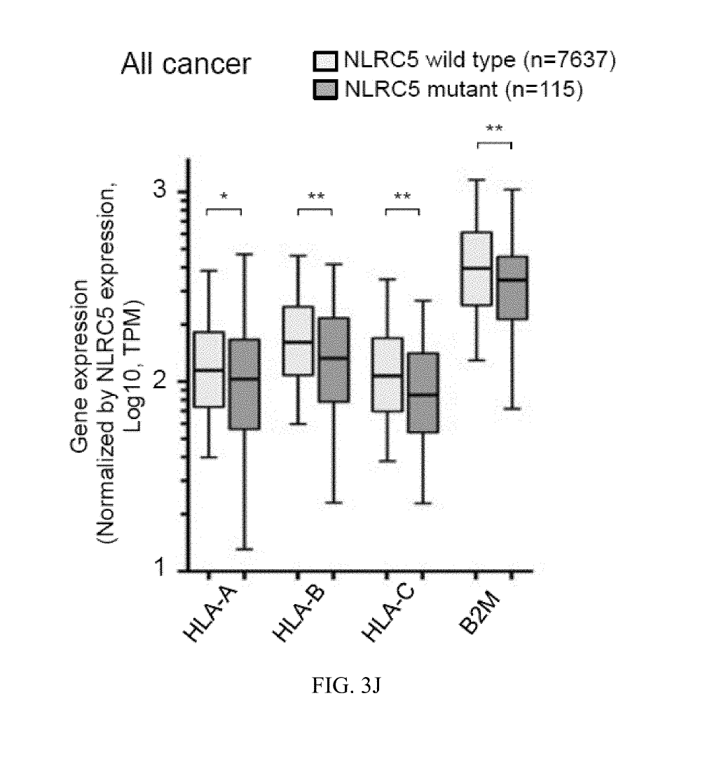

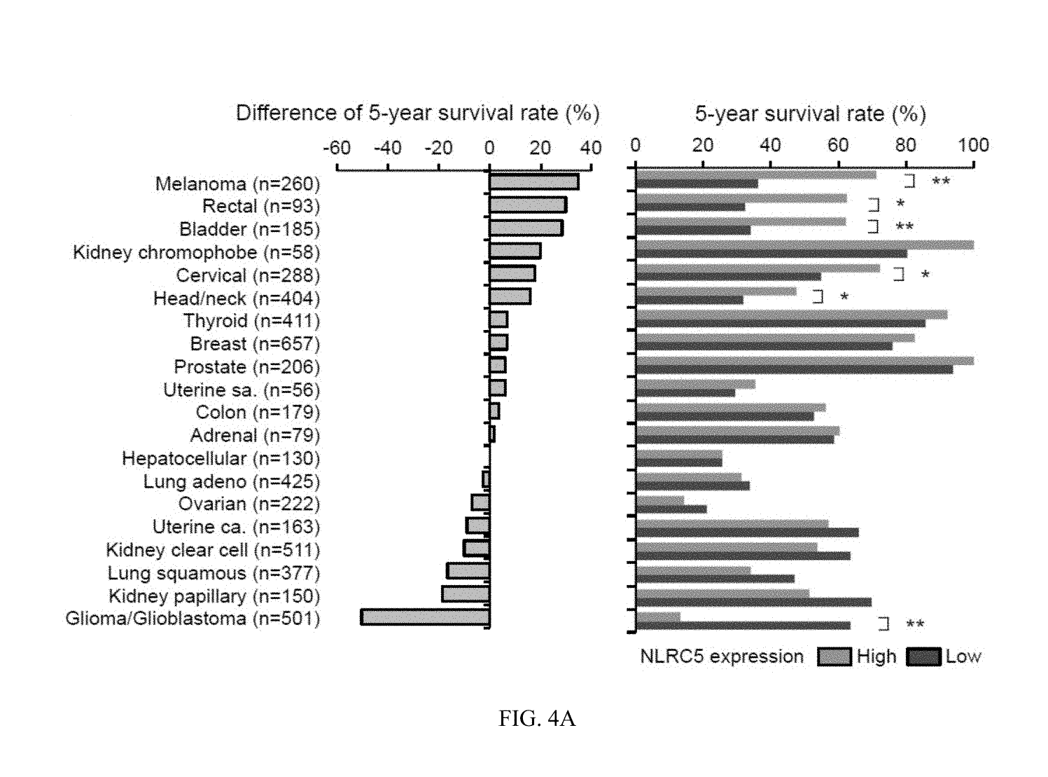

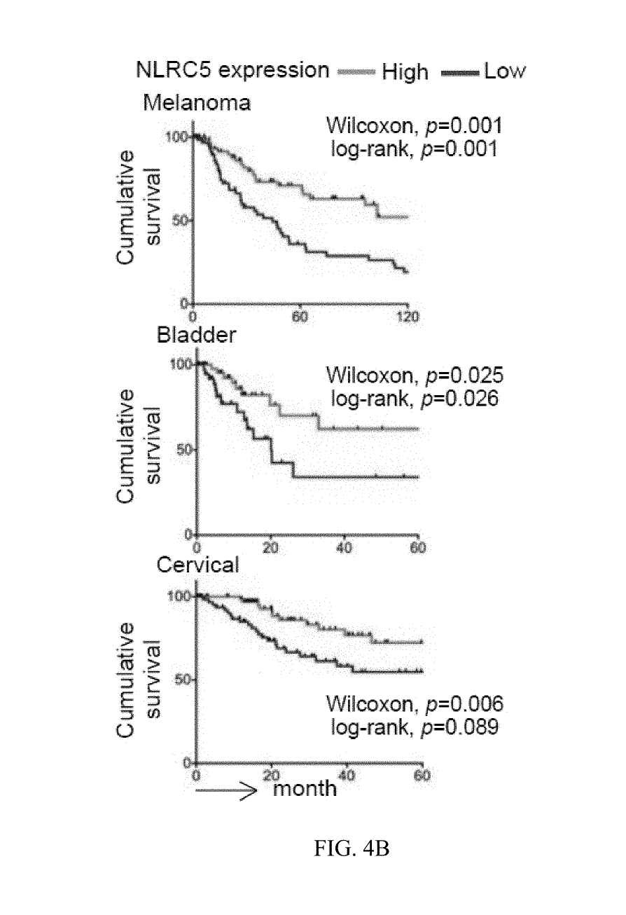

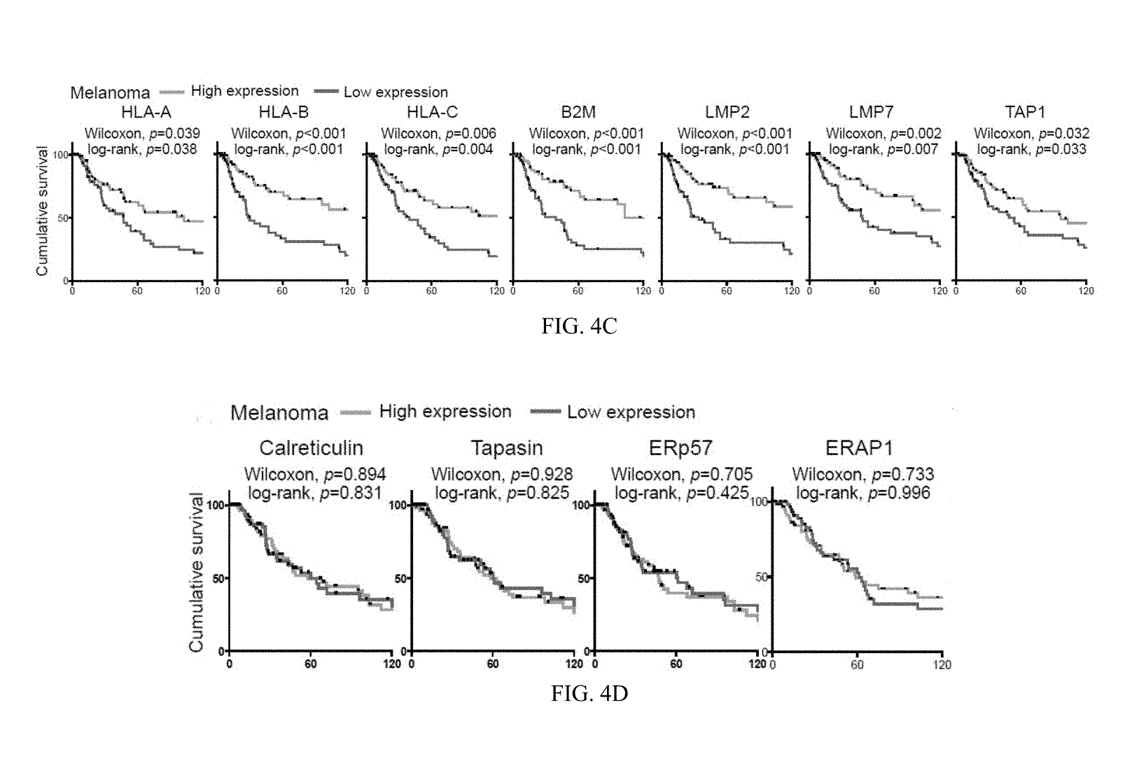

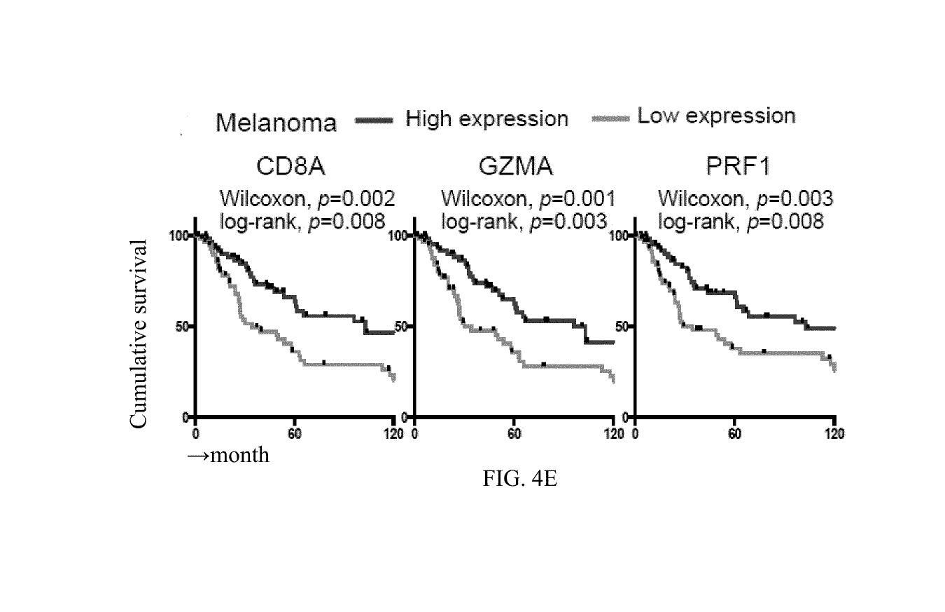

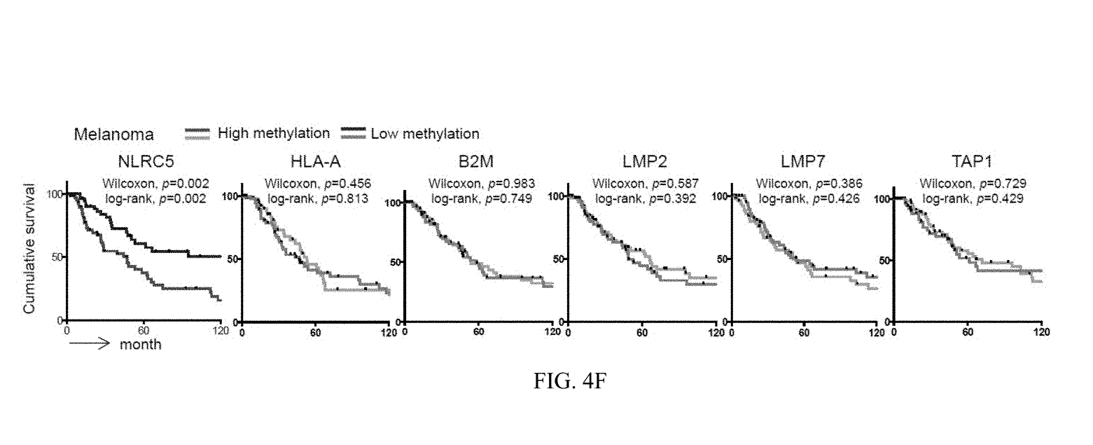

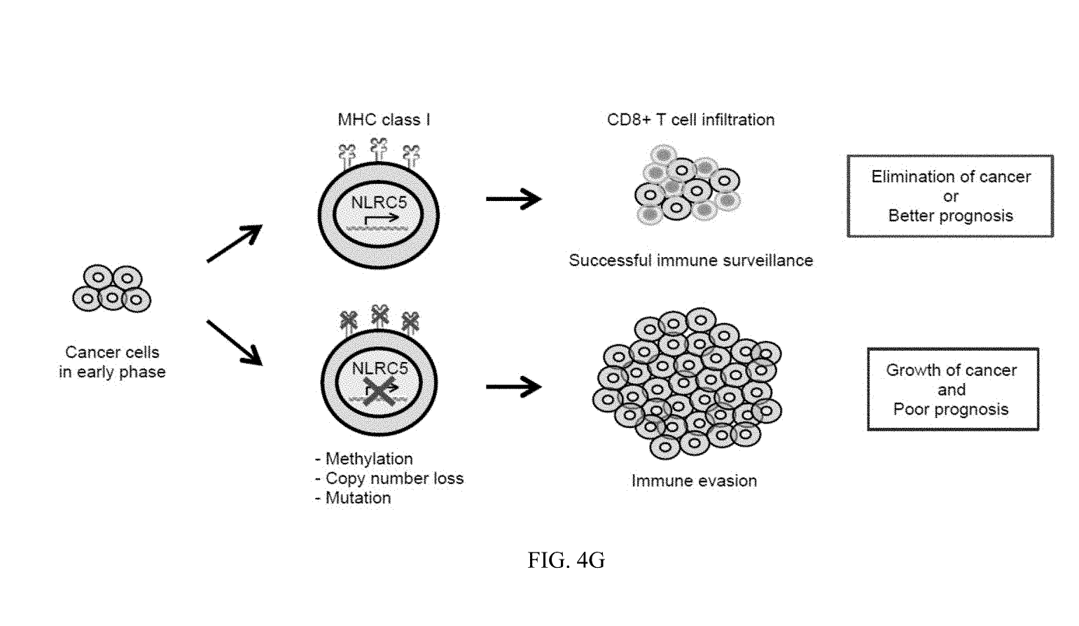

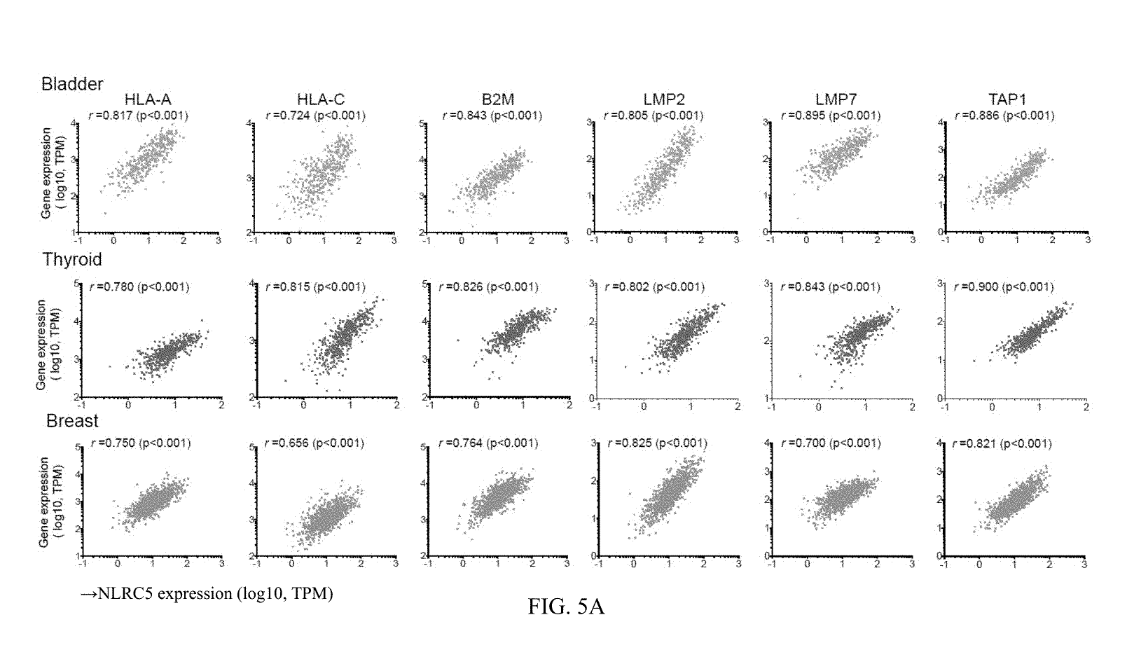

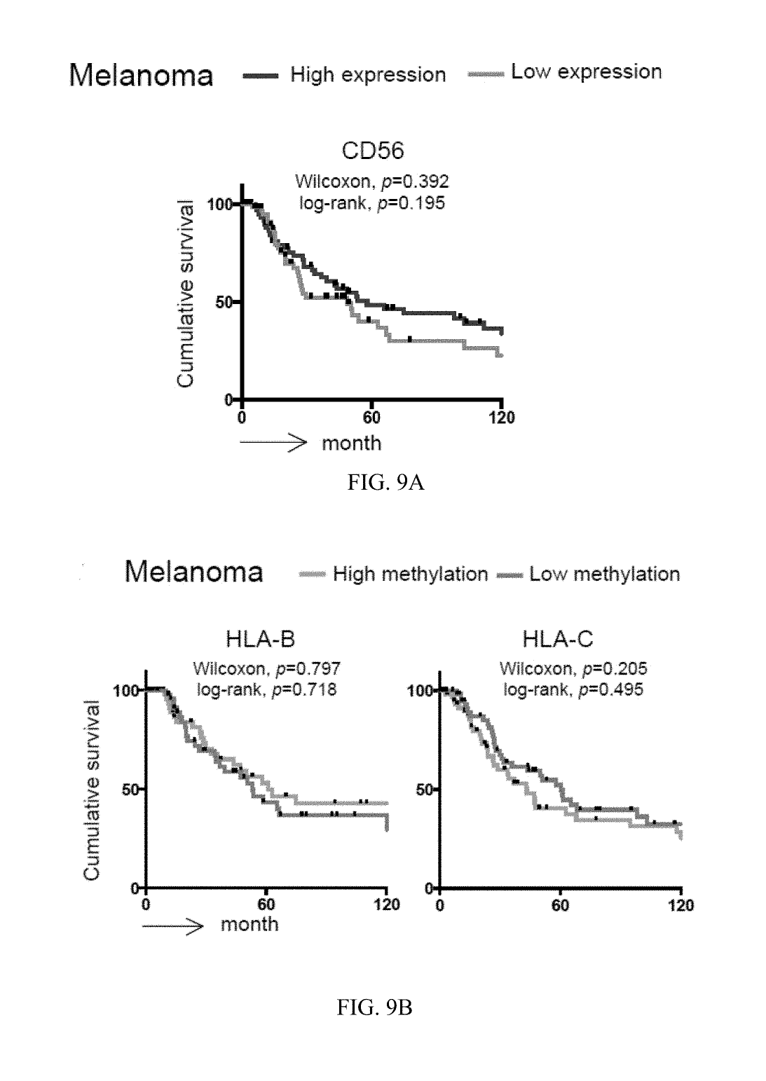

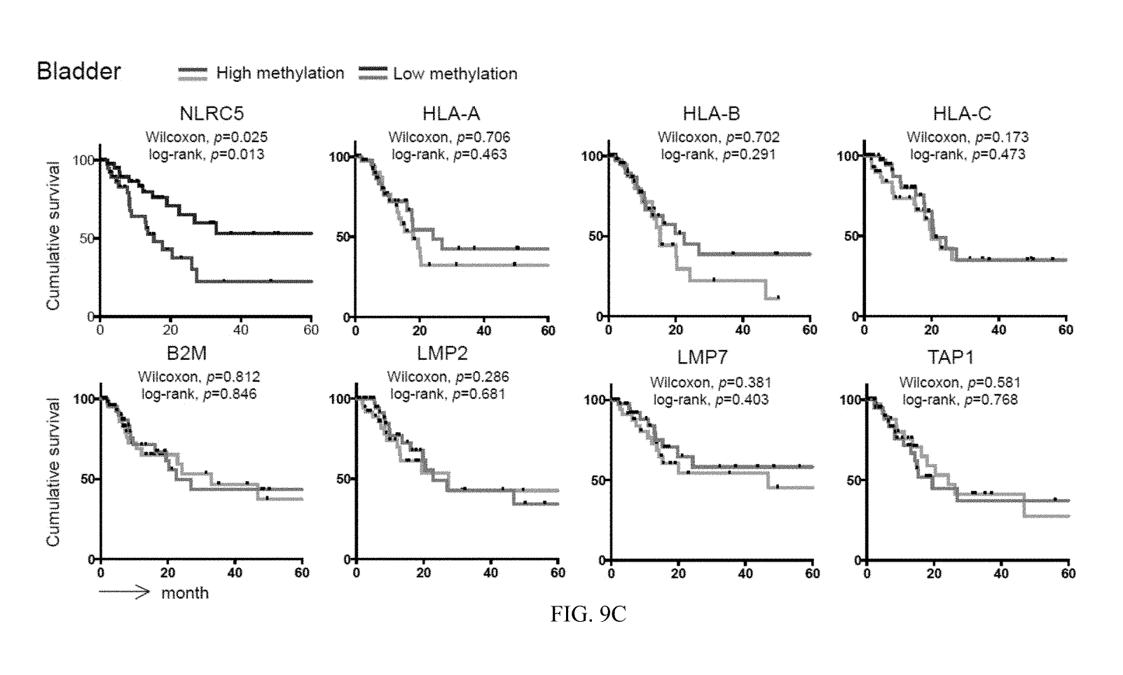

As such, the invention provides NLRC5 as a major target for immune evasion by cancer cells (FIG. 4G). During oncogenic transformation and cancer evolution, cancer cells need to develop ways to escape from the host immune system to sustain development, growth, invasion and metastasis. Reduction, alteration or total loss of tumor antigen expression is critical to avoid killing via activation of cytotoxic CD8⁺ T cells, and that can be achieved by at least three mechanisms: 1) lack of expression of tumor antigen; 2) loss of MHC class I molecules; and 3) impaired function or expression of genes in the class I antigen presentation pathway such as in the immunoproteasome or class I peptide loading complex at the endoplasmic reticulum. As a master regulator of MHC class I genes, impaired function or expression of NLRC5 affects the latter two steps concurrently, thus making NLRC5 an attractive target for cancer cells to evade CD8⁺ T cell-dependent immune responses. Indeed, the expression of NLRC5 correlates with markers for cytotoxic CD8⁺ T cell activity and is associated with better prognosis with prolonged patient survival in multiple cancers (FIGS. 1E, F and FIGS. 4A, B). Further, the expression of NLRC5-dependent but not independent genes involved in MHC class I antigen presentation is associated with cancer patient survival, further supporting the significance of the NLRC5-dependent MHC class I transactivation pathway in anti-tumor immunity (FIGS. 4C, D). Several lines of evidence demonstrated that cancer cells have evolved to preferentially target nlrc5 for immune evasion during their evolution. First, the nlrc5 promoter is highly methylated, more than any other gene in the MHC class I pathway (FIG. 2H). Second, the methylation-mediated suppression of gene expression is most effective for nlrc5 (FIGS. 2B, I and J). Third, copy number loss is most frequently observed in nlrc5 among all MHC class I related genes (FIG. 3B). The methylation status of nlrc5, but not of other MHC class I and related genes, was associated with changes in patient survival of melanoma and bladder cancer (FIG. 4F and FIGS. 9B, C). These data signify NLRC5 as a major target of immune evasion in cancers. In addition to promoter methylation and copy number loss, NLRC5-targeted immune evasion is achieved by mutations in nlrc5, which resulted in the reduction of expression of both MHC class I and related genes. Although nlrc5 is expressed in both cancer and infiltrating T cells, it is unlikely that aberrant promoter methylation, copy number loss and mutations in nlrc5 occur in normal infiltrating cells. Therefore, genetic as well as epigenetic alterations within the cancer cells impact MHC class I-dependent immune responses through altered activity of NLRC5. Since high expression and low methylation of nlrc5 correlate with better survival of cancer patients, nlrc5 expression and methylation status are useful biomarkers for patient prognosis and survival in multiple cancers. Also, the invention indicates that NLRC5 is an attractive therapeutic target. Checkpoint blockade immunotherapy such as anti-CTLA4 or anti-PD-1/PD-L1 therapy has emerged as a leading cancer treatment, although the efficacy of these therapies is hampered when cancer cells successfully evade immune responses. As such, the invention provides therapeutics targeting nlrc5, NLRC5 mRNA or NLRC5 protein to complement immunotherapies by breaking immune evasion in cancer.

As noted above, cancer cells can evade the immune system of a subject by reducing the expression of nlrc5. Accordingly, an embodiment of the invention provides a method of identifying a subject as having a cancer that is likely or not likely to evade the immune system of the subject or provide information regarding prognosis or the likelihood of responding to a treatment and optionally, treating the subject. The method comprises the steps of:

(a) determining the amount of NLRC5 mRNA or NLRC5 protein in:
i) a test sample obtained from the subject, and
ii) optionally, a control sample;

(b) optionally, obtaining one or more reference values for the amount of NLRC5 mRNA or NLRC5 protein; and
(i) identifying the subject as having a cancer that is likely to evade the immune system of the subject based on the amount of NLRC5 mRNA or NLRC5 protein in the test sample as compared to the control sample or the reference value and, optionally, administering a first therapy and/or a second therapy to the subject to treat the cancer, or
(ii) identifying the subject as having a cancer that is not likely to evade the immune system of the subject based on the amount of NLRC5 mRNA or NLRC5 protein in the test sample as compared to the control sample or the reference value and, optionally, administering a first therapy to the subject to treat the cancer and/or withholding the administration of the second therapy to the subject.

In various embodiments, the amount of neoantigen, mutation number and the expression of immune response gene including but not limited to CLTA4 PD1, PD-L1 and PD-L2 in the test sample from the subject is also obtained and compared against a control sample and subjects having elevated levels these variables are treated with a first therapy and/or withholding the administration of a second therapy.

In any aspects or embodiments disclosed herein, subjects having elevated levels the aforementioned variables show an increase of at least 5%, 10%, 20%, 30%, 40%, 50%, 60%, 70%, 80%, 90%, or 100% of the variables relative to expression levels in a control sample. Further, in any aspects or embodiments disclosed herein, subjects having lower levels the aforementioned variables show a decrease of at least 5%, 10%, 20%, 30%, 40%, 50%, 60%, 70%, 80%, 90%, or 100% of the variables relative to expression levels in a control sample. Appropriate first and second therapies are described later in this application.

In one embodiment, the step of identifying the subject as having a cancer that is likely or not likely to evade the immune system of the subject (thus, is likely or not likely to show better prognosis and/or better response to cancer therapies) depends on the amount of NLRC5 mRNA, or NLRC5 protein by themselves or depends on the amount of NLRC5 mRNA or protein, in combination with other variables, including, but not limited to neoantigen load, mutation number, and expression of genes involved in immune responses, including but not limited to CTLA4, PD1, PD-L1 and PD-L2 in a test sample. For example, if the amount of NLRC5 mRNA or NLRC5 protein by themselves or in combination with other variables, including, but not limited to neoantigen load, mutation number, and expression of genes involved in immune responses, including but not limited to CTLA4, PD1, PD-L1 and PD-L2 in a test sample of cancer tissues is lower in the cancer patient group, the subject is identified as having a cancer that is likely to evade the immune system of the subject (thus, is likely to show poor prognosis and/or poor response to cancer therapies). Alternately, if the amount of NLRC5 mRNA or NLRC5 protein by themselves or in combination with other variables, including, but not limited to neoantigen load, mutation number, and expression of genes involved in immune responses, including but not limited to CTLA4, PD1, PD-L1 and PD-L2 is higher in the cancer patient group, the subject is identified as having a cancer that is not likely to evade the immune system of the subject (thus, is likely to show good prognosis and/or good response to cancer therapies).

The reference value for the amount of NLRC5 mRNA or NLRC5 protein may indicate the amount of NLRC5 mRNA or NLRC5 protein associated with a cancer that is likely to evade the immune system of the subject. Alternately, the reference value corresponding to the amount of NLRC5 mRNA or NLRC5 protein may indicate the amount of NLRC5 mRNA or NLRC5 protein associated with a cancer that is not likely to evade the immune system of the subject. Accordingly, the step of identifying the subject as having a cancer which is likely or not likely to evade the immune system of the subject depends on the amount of NLRC5 mRNA or NLRC5 protein by themselves or in combination with other variables, including, but not limited to neoantigen load, mutation number, and expression of genes involved in immune responses, including but not limited to CTLA4, PD1, PD-L1 and PD-L2, in a test sample as compared to the reference value depending on whether the reference value indicates the amount of NLRC5 mRNA, NLRC5 protein, by themselves or in combination with other variables, including, but not limited to neoantigen load, mutation number, and expression of genes involved in immune responses, including but not limited to CTLA4, PD1, PD-L1 and PD-L2 associated with a cancer that is likely or not likely to evade the immune system of the subject (thus, is likely or not likely to show better prognosis and/or better response to cancer therapies).

In one embodiment, if the amount of NLRC5 mRNA or NLRC5 protein by themselves or in combination with other variables, including, but not limited to neoantigen load, mutation number, and expression of genes involved in immune responses, including but not limited to CTLA4, PD1, PD-L1 and PD-L2 in a test sample from a subject indicates that the subject has a cancer that not likely to evade the immune system of the subject, additional biomarkers described herein, for example, the activity of NLRC5 protein, the level of methylation of nlrc5 or a portion thereof and/or the copy number of nlrc5, are tested to identify whether the subject has a cancer that is likely or not likely to evade the immune system of the subject (thus, is likely or not likely to show better prognosis and/or better response to cancer therapies).

Various techniques are known to a person of ordinary skill in the art to determine the mRNA amount of NLRC5 and genes involved in immune responses, including but not limited to CTLA4, PD1, PD-L1 and PD-L2 in a sample. Non-limiting examples of such techniques include microarray analysis, real-time polymerase chain reaction (PCR), Northern blot, in situ hybridization, solution hybridization, quantitative reverse transcription PCR (qRT-PCR) or RNAseq. Methods of carrying out these techniques are routine in the art. Additional methods of determining the amount of NLRC5 mRNA in a sample are also well-known to a person of ordinary skill in the art and such embodiments are within the purview of the invention.

Also, various techniques are well known to a person of ordinary skill in the art to determine the level of NLRC5 protein in a sample. Non-limiting examples of such techniques include protein array analysis, Western blot analysis, flow cytometry (for example, by setting an intensity cut-off value), enzyme-linked immunosorbent assay (ELISA) and radioimmunoassay (MA). Methods of carrying out these techniques are routine in the art. Additional methods of determining the level of NLRC5 protein in a sample are also well-known to a person of ordinary skill in the art and such embodiments are within the purview of the invention.

Also techniques are known to a person of ordinary skill in the art to determine the neo antigen load and mutation number. Non-limiting examples of such techniques include exosome sequencing and shot-gun sequencing. Methods of carrying out these techniques are routine in the art.

A further embodiment of the invention provides a kit comprising reagents to carry out the methods of the current invention. In one embodiment, the kit comprises primers or probes specific for NLRC5 mRNA. Reagents for treating the samples, for example, deproteination, degradation of DNA, or removal of other impurities, can also be provided in the kit.

Another mechanism by which a cancer cell may evade the immune system of a subject is a mutation that reduces the activity of NLRC5 protein, particularly the transcription activity of NLRC5/transcription factor complex for its target genes. Accordingly, an embodiment of the invention provides a method of identifying a subject as having a cancer that is likely or not likely to evade the immune system of the subject and, optionally, treating the subject, the method comprising the steps of:

(a) determining the transcription factor activity of NLRC5 protein in:
i) a test sample obtained from the subject, and
ii) optionally, a control sample;

(b) optionally, obtaining one or more reference values for the transcription factor activity of NLRC5 protein; and
(i) identifying the subject as having the cancer that is likely to evade the immune system of the subject based on the transcription factor activity of NLRC5 protein in the test sample as compared to the control sample or the reference value and optionally, administering a first therapy and/or a second therapy to the subject to treat the cancer, or
(ii) identifying the subject as having a cancer that is not likely to evade the immune system of the subject based on the transcriptional activity of NLRC5 protein in the test sample as compared to the control sample or the reference value and, optionally, administering a first therapy to the subject to treat the cancer and/or withholding the administration of the second therapy to the subject.

Appropriate first and second therapies are described later in this application.

As used herein, the phrase “the transcriptional activity of NLRC5/transcription factor complex” refers to the ability of NLRC5 protein to induce the transcription of its target genes encoding HLA-A, HLA-B, HLA-C, HLA-E, B2M, LMP2, LMP7, LMP9 and TAP1.

In one embodiment, the step of identifying the subject as having a cancer which is likely or not likely to evade the immune system of the subject (thus, is likely or not likely to show better prognosis and/or better response to cancer therapies) depends on the transcriptional activity of NLRC5/transcription factor complex in the test sample. For example, if the transcriptional activity of NLRC5/transcription factor complex in a test sample is lower than the transcriptional activity of NLRC5 protein in a control sample, the subject is identified as having a cancer which is likely to evade the immune system of the subject (thus, is likely to show poor prognosis and/or poor response to cancer therapies). If levels of neoantigen load, mutation number, or expression of genes involved in immune responses, including but not limited to CTLA4, PD1, PD-L1 and PD-L2 are also low, in comparison to the control sample, then the subject is also likely to have a cancer likely to have a poor response to cancer therapies. Alternately, if the transcriptional activity of NLRC5/transcription factor complex in a test sample is equal to or higher than the transcriptional activity of NLRC5/transcription factor complex in a control sample, the subject is identified as having a cancer which is not likely to evade the immune system of the subject (thus, is likely or not likely to show better prognosis and/or better response to cancer therapies). If levels of neoantigen load, mutation number, or expression of genes involved in immune responses, including but not limited to CTLA4, PD1, PD-L1 and PD-L2 are also high, in comparison to the control sample, then the subject is also likely to have a cancer likely to have a better response to cancer therapies.

A reference value for the transcriptional activity of NLRC5/transcription factor complex may indicate the transcriptional activity of NLRC5 protein associated with a cancer that is likely to evade the immune system of the subject (thus, is likely to show poor prognosis and/or poor response to cancer therapies). Alternately, a reference value for the transcriptional activity of NLRC5/transcription factor complex may indicate the transcription factor activity of NLRC5 protein associated with a cancer that is not likely to evade the immune system of the subject (thus, is likely to show good prognosis and/or good response to cancer therapies). Accordingly, the step of identifying the subject as having a cancer which is likely or not likely to evade the immune system of the subject (thus, is likely or not likely to show better prognosis and/or better response to cancer therapies) depends on the transcriptional activity of NLRC5 protein in a test sample as compared to the reference value depending on whether the reference value indicates the transcriptional activity of NLRC5 protein associated with a cancer that is likely or not likely to evade the immune system of the subject (thus, is likely or not likely to show better prognosis and/or better response to cancer therapies). Similarly, the response of a patient to a cancer therapy is likely to be better if levels of neoantigen are higher, in comparison to the control sample.

A number of techniques are known to a person of ordinary skill in the art to determine the transcriptional activity of a protein. Typically, a transcriptional activity of a protein for its target gene can be determined based on the ability of the protein to bind to specific DNA sequences present in a promoter of a target gene and/or recruit transcription factor machinery to the promoter of the target gene. Non-limiting examples of the techniques to determine the transcriptional activity of a protein include chromatin immunoprecipitation assay and reporter gene (for example, luciferase) expression assay. Additional examples of techniques used to determine the transcriptional activity of a protein are well-known to a person of ordinary skill in the art and such embodiments are within the purview of the invention.

A further embodiment of the invention provides a kit comprising reagents to conduct an assay to determine the transcriptional activity of NLRC5 protein. The kit can include reagents to clone a target gene promoter into the expression vectors, culture the cells, transfect the cultured cells with the expression vectors and assay the activity of a reporter gene.

As noted above, a mechanism by which a cancer cell evades the immune system of a subject is a mutation that reduces the activity of NLRC5 protein, particularly the transcriptional activity of NLRC5/transcription factor complex for its target genes. Accordingly, an embodiment of the invention provides a method of identifying a subject as having a cancer that is likely or not likely to evade the immune system of a subject (thus, is likely or not likely to show better prognosis and/or better response to cancer therapies) and, optionally, treating the subject, the method comprising the steps of:

(a) determining the sequence of the protein coding region of nlrc5 or a portion thereof in a test sample obtained from the subject; and

(b) optionally, determining the sequence of NLRC5 protein encoded by nlrc5 or a portion thereof in the test sample and, optionally, determining the activity of a wild-type NLRC5 protein and the NLRC5 protein encoded by nlrc5 in the test sample, and
(i) identifying the subject as having a cancer that is likely to evade the immune system of the subject (thus, is likely to show poor prognosis and/or poor response to cancer therapies) if nlrc5 or NLRC5 protein in the test sample contains a mutation that reduces the transcriptional activity of NLRC5/transcription factor complex in the test sample as compared to the wild-type NLRC5 protein and, optionally, administering a first therapy and/or a second therapy to the subject to treat the cancer, or
(d) identifying the subject as having a cancer that is not likely to evade the immune system of the subject if nlrc5 or NLRC5 protein in the test sample does not contain a mutation or contains a mutation that does not affect or increases the transcription factor activity of NLRC5 protein in the test sample as compared to the wild-type NLRC5 protein (thus, is likely to show good prognosis and/or good response to cancer therapies) and, optionally, administering a first therapy to the subject to treat the cancer and/or withholding the administration of the second therapy to the subject.

This method may also be used in combination with determining neoantigen levels or mutation numbers in a subject with higher levels of neoantigen load, mutation number, or expression of genes involved in immune responses, including but not limited to CTLA4, PD1, PD-L1 and PD-L2 in a subject with higher levels of those parameters, as compared to control samples, indicating that the subject is likely to have a better response to a cancer therapy. Appropriate first and second therapies are described later in this application.

In one embodiment, the subject is a human and the protein coding region of the wild-type nlrc5 has the sequence of SEQ ID NO: 1 and the wild-type NLRC5 protein has the sequence of SEQ ID NO: 2.

In one embodiment, the sequence of NLRC5 protein is determined, for example, by protein sequencing, without determining the sequence of nlrc5.

Various methods for sequencing nlrc5 gene or a portion thereof as well as protein sequencing are known to a person of ordinary skill in the art and such embodiments are within the purview of the invention.

The transcriptional activity, methods of determining the transcriptional activity and the reference values for the transcriptional activity of the NLRC5 protein are described elsewhere in this disclosure and are relevant to this embodiment of the invention.

The step of identifying the subject as having a cancer which is likely or not likely to evade the immune system of the subject (thus, is likely or not likely to show better prognosis and/or better response to cancer therapies) depends on whether the mutation in the NLRC5 protein affects the transcriptional activity of NLRC5 protein in the test sample. For example, if the transcriptional activity of NLRC5 protein in a test sample is lower than the wild-type NLRC5 protein, the subject is identified as having a cancer which is likely to evade the immune system of the subject (thus, is likely to show poor prognosis and/or poor response to cancer therapies). Alternately, if the transcription factor activity of NLRC5 protein in a test sample is equal to or higher than the transcription factor activity of the wild-type NLRC5 protein, the subject is identified as having a cancer which is not likely to evade the immune system of the subject (thus, is likely to show good prognosis and/or good response to cancer therapies).

In one embodiment, a mutation that reduces the activity of the NLRC5 protein as compared to the wild-type NLRC5 protein includes: L181P, R262C, R550W, A737D, H1717fs*29, R1830C, or Q1847*. Accordingly, a subject having one or more of the following mutations in the NLRC5 protein as compared to the wild-type NLRC5 protein is identified as having a cancer that is likely to evade the immune system of the subject (thus, is likely to show poor prognosis and/or poor response to cancer therapies): L181P, R262C, R550W, A737D, H1717fs*29, R1830C, and Q1847*.

In another embodiment, a mutation that does not reduce or increases the activity of the NLRC5 protein as compared to the wild-type NLRC5 protein includes: R386W, S496F, R574H, D884N, T1173M, or A1512T. Accordingly, a subject having one or more of the following mutations in the NLRC5 protein as compared to the wild-type NLRC5 protein is identified as having a cancer that is not likely to evade the immune system of the subject (thus, is likely to show good prognosis and/or good response to cancer therapies): R386W, S496F, R574H, D884N, T1173M, and A1512T.

In an embodiment, if the NLRC5 protein in a test sample from the subject does not contain a mutation or contains a mutation that does not reduce the activity of the NLRC5 protein compared to a wild-type NLRC5 protein, additional biomarkers described herein, for example, the amount of NLRC5 mRNA, the amount of NLRC5 protein, the activity of NLRC5 protein and/or the level of methylation of nlrc5 or a portion thereof, are determined to identify the subject as having a cancer that is likely or not likely to evade the immune system of the subject.

A further embodiment of the invention provides a kit comprising reagents to determine the sequence of nlrc5 or a portion thereof or the NLRC5 protein or a portion thereof. The kit can also comprise reagents to conduct an assay to determine the transcription factor activity of the NLRC5 protein. The kit can include reagents to clone a target gene promoter into the expression vectors, culture the cells, transfect the cultured cells with the expression vectors and assay the activity of a reporter gene.

Another mechanism by which a cancer cell reduces the expression of nlrc5 involves methylation of nlrc5 of a portion thereof, particularly, the nlrc5 promoter having the sequence of SEQ ID NO: 4 or 5.

Accordingly, an embodiment of the invention provides a method of identifying a subject as having a cancer that is likely or not likely to evade the immune system of the subject (thus, is likely or not likely to show better prognosis and/or better response to cancer therapies), and, optionally, treating the subject, the method comprising the steps of:

(a) determining the level of methylation of nlrc5 or a portion thereof in:
i) a test sample obtained from the subject, and
ii) optionally, a control sample; and

(b) optionally, obtaining one or more reference values for the levels of methylation of nlrc5 or a portion thereof; and
(i) identifying the subject as having a cancer that is likely to evade the immune system of the subject (thus, is likely to show poor prognosis and/or poor response to cancer therapies) based on the level of methylation of nlrc5 or a portion thereof in the test sample as compared to the control sample or the reference value and, optionally, administering a first therapy and/or a second therapy to the subject to treat the cancer, or
(ii) identifying the subject as having a cancer that is not likely to evade the immune system of the subject (thus, is likely to show good prognosis and/or good response to cancer therapies) based on the level of methylation of nlrc5 or a portion thereof in the test sample as compared to the control sample or the reference value and, optionally, administering a first therapy to the subject to treat the cancer and/or withholding the administration of the second therapy to the subject.

This method may also be used in combination with determining neoantigen load, mutation number, or expression of genes involved in immune responses, including but not limited to CTLA4, PD1, PD-L1 and PD-L2 in a subject with higher levels of those parameters, as compared to control samples, indicating that the subject is likely to have a better response to a cancer therapy. Appropriate first and second therapies are described later in this application.

As used herein, the phrase “level of methylation” as applied to nlrc5 or a portion thereof refers to whether one or more cytosine residues present in one or more CpG sites within a sequence of interest have or do not have a methylation group.

In one embodiment, the level of methylation is indicated as a beta-value. The beta-value is a number between zero and one. A value of zero indicates that every copy of the CpG sites within a sequence of interest in the sample is unmethylated, whereas a value of one indicates that every copy of the CpG sites within a sequence of interest is methylated. For example, in a fluorescent methylation/demethylation specific probe-based assay, such as Illumina Infinium Human DNA Methylation 450, the beta-value provides a ratio between methylated probe intensity and total probe intensities (sum of methylated and demethylated probe intensities).

A reference value corresponding to the level of methylation of nlrc5 or a portion thereof may indicate the level of methylation associated with a cancer that is likely to evade the immune system of the subject (thus, is likely to show poor prognosis and/or poor response to cancer therapies). Alternately, a reference value corresponding to the level of methylation of nlrc5 or a portion thereof may indicate the level of methylation associated with a cancer that is not likely to evade the immune system of the subject (thus, is likely to show good prognosis and/or good response to cancer therapies).

The step of identifying the subject as having a cancer which is likely or not likely to evade the immune system of the subject (thus, is likely or not likely to show better prognosis and/or better response to cancer therapies) depends on the level of methylation of nlrc5 or a portion thereof in the test sample. For example, if the level of methylation of nlrc5 or a portion thereof in a test sample is higher than the level of methylation of nlrc5 or a portion thereof in a control sample, the subject is identified as having a cancer which is likely to evade the immune system of the subject (thus, is likely to show poor prognosis and/or poor response to cancer therapies). Alternately, if the level of methylation of nlrc5 or a portion thereof in a test sample is equal to or lower than the level of methylation of nlrc5 or a portion thereof in a control sample, the subject is identified as having a cancer which is not likely to evade the immune system of the subject (thus, is likely to show good prognosis and/or good response to cancer therapies).

In one embodiment, the beta-value for methylation of nlrc5 or a portion thereof in a test sample of above 0.2, 0.3 or 0.4 indicates that the subject has a cancer which is likely to evade the immune system of the subject, whereas the beta-value for methylation of nlrc5 or a portion thereof in a test sample of below 0.2, 0.3 or 0.4 indicates that the subject has a cancer which is not likely to evade the immune system of the subject. In a specific embodiment, the beta-value above 0.2, 0.3 or 0.4 for methylation of a portion of nlrc5 having the sequence of SEQ ID NO: 5 or 6 in a test sample indicates that the subject has a cancer which is likely to evade the immune system of the subject, whereas the beta-value below 0.2, 0.3 or 0.4 for methylation of a portion of nlrc5 having the sequence of SEQ ID NO: 5 or 6 indicates that the subject has a cancer which is not likely to evade the immune system of the subject.

In one embodiment, if the level of methylation of nlrc5 or a portion thereof in a test sample from a subject indicates that the subject has a cancer that not likely to evade the immune system of the subject (thus, is likely to show poor prognosis and/or poor response to cancer therapies), additional biomarkers described herein, for example, amount of NLRC5 mRNA, amount of NLRC5 protein, activity of NLRC5 protein or the copy number of nlrc5, are tested to identify whether the subject has a cancer that is likely or not likely to evade the immune system of the subject (thus, is likely or not likely to show better prognosis and/or better response to cancer therapies).

Various techniques are known to a person of ordinary skill in the art to determine the level of methylation of a genomic site in a sample. Non-limiting examples of such techniques include bisulfite conversion, digestion by restriction enzymes followed by polymerase chain reaction (Combined Bisulfite Restriction Analysis, COBRA), direct sequencing, cloning and sequencing, pyrosequencing, mass spectrometry analysis, probe/microarray-based assay, methylation-sensitive single-strand confirmation analysis, high resolution melting analysis, methylation-sensitive single-nucleotide primer extension, base-specific cleavage/MALDI-TOF, the Hpall tiny fragment Enrichment by Ligation-mediated PCR (HELP) assay, ChIP-on-ChIP, restriction landmark genomic scanning, methylated DNA immunoprecipitation, molecular break light assay for DNA adenine methyltransferase activity and methyl-sensitive Southern blotting.

Certain techniques of determining methylation of genomic sites are described in Eads et al., Xiong et al., Paul et al., Warnecke et al., Tost et al., and Ehrich et al., the contents of which are herein incorporated in their entirety. Additional techniques for determining DNA methylation of one or more sites in the genomic DNA of a sample are well-known to a person of ordinary skill in the art and such techniques are within the purview of the invention.

A further embodiment of the invention provides a kit comprising reagents to determine the level of methylation of nlrc5 or a portion thereof. The kit can include reagents for isolation of genomic DNA from a sample, reagents to treat the genomic DNA, for example, bisulfite treatment, specific primers to analyze the level of methylation of nlrc5 or a portion thereof and reagents for PCR amplification of nlrc5 or a portion thereof.

Yet another mechanism by which a cancer cell may have a reduced expression of nlrc5 involves reduction in the gene copy number of nlrc5. Accordingly, an embodiment of the invention provides a method of identifying a subject as having a cancer that is likely or not likely to evade the immune system of the subject (thus, is likely or not likely to show better prognosis and/or better response to cancer therapies), and, optionally, treating the subject, the method comprising the steps of:

(a) determining the copy number of nlrc5 in a test sample obtained from the subject; and
(i) identifying the subject as having a cancer that is likely to evade the immune system of the subject based on the copy number of nlrc5 being below about two in the test sample (thus, is likely to show poor prognosis and/or poor response to cancer therapies) and, optionally, administering a first therapy and/or a second therapy to the subject to treat the cancer, or
(ii) identifying the subject as having a cancer that is not likely to evade the immune system of the subject based on the copy number of nlrc5 being above about two in the test sample (thus, is likely to show good prognosis and/or good response to cancer therapies) and, optionally, administering a first therapy to the subject to treat the cancer and/or withholding the administration of the second therapy to the subject.

This method may also be used in combination with determining neoantigen load, mutation number, or expression of genes involved in immune responses, including but not limited to CTLA4, PD1, PD-L1 and PD-L2 in a subject with higher levels of those parameters, as compared to control samples, indicating that the subject is likely to have a better response to a cancer therapy. Appropriate first and second therapies are described later in this application.

As used herein, the term “copy number” as applied to nlrc5 refers to the number of copies of nlrc5 that are present in the genome of a cancer cell of a subject. When referring to a group of cancer cells, the “copy number” as applied to nlrc5 refers to the average number of copies of nlrc5 that are present in the genomes of the cancer cells of a subject.

In a normal mammalian cell, the copy number of nlrc5 is two. Loss of one or both copies may occur in a cancer cell that enables the cell to evade the immune system of a subject. A copy number for a group of cells can be expressed as an average copy number which may not be an integer.

In one embodiment, when the term “about” is used to indicate the copy number, a 10% variation is permitted. Therefore, a copy number of above 1.8 (average copy number) for a sample of cells in a subject indicates that the subject has a cancer that is not likely to evade the immune system of the subject, whereas a copy number of below 1.8 (average copy number) for a sample of cells in a subject indicates that the subject has a cancer that is not likely to evade the immune system of the subject.

In one embodiment, if the copy number of nlrc5 in cancer cells of a subject is determined to be about two or more, additional biomarkers described herein, for example, the amount of NLRC5 mRNA, the amount of NLRC5 protein, the activity of NLRC5 protein, the level of methylation of nlrc5 or a portion thereof or a mutation in nlrc5, are determined to identify the subject as having a cancer that is likely or not likely to evade the immune system of the subject.

The gene copy number of nlrc5 can be determined using PCR, RT-PCR, quantitative PCR or fluorescent in-situ hybridization using a labeled probe. Additional techniques for determining gene copy number are known to a person of ordinary skill in the art and such embodiments are within the purview of the invention.

A further embodiment of the invention provides a kit comprising reagents to determine the copy number of nlrc5. The kit can include reagents to isolate genomic DNA from a sample and treat the genomic DNA and specific primers and/or probes used to analyze the copy number of nlrc5.

To practice the methods described herein for identifying a subject as having a cancer that is likely or not likely to evade the immune system of the subject (thus, is likely or not likely to show better prognosis and/or better response to cancer therapies), the control samples can be obtained from one or more of the following:

a) an individual belonging to the same species as the subject and not having cancer,

b) an individual belonging to the same species as the subject and known to have a cancer that does not evade the immune system of the subject, or

c) the subject prior to getting the cancer.

Additional examples of control samples and appropriate experimental designs based on the selected control samples are well-known to a person of ordinary skill in the art and such embodiments are within the purview of the current invention.

In certain embodiments, a control sample and a test sample are obtained from the same type of organ or tissue. Non-limiting examples of the organs or tissues that can be used as samples include placenta, brain, eyes, pineal gland, pituitary gland, thyroid gland, parathyroid glands, thorax, heart, lung, esophagus, thymus gland, pleura, adrenal glands, appendix, gall bladder, urinary bladder, large intestine, small intestine, kidneys, liver, pancreas, spleen, stoma, ovaries, uterus, testis, skin, blood or buffy coat sample of blood. Additional examples of organs and tissues are well-known to a person of ordinary skill in the art and such embodiments are within the purview of the invention.

For the purpose of the invention, a reference value for a biomarker associated with a cancer that is likely to evade the immune system of a subject (thus, is likely to show poor prognosis and/or poor response to cancer therapies) may be obtained based on samples obtained from patients known to have a cancer that evades the immune systems of the patients. Similarly, a reference value for a biomarker associated with a cancer that is not likely to evade the immune system of a subject (thus, is likely to show good prognosis and/or good response to cancer therapies) may be obtained based on samples obtained from patients known to have a cancer that does not evade the immune systems of the patients.

In one embodiment, tissues from a group of patients having a cancer are obtained and the values for a biomarker are determined in these tissues. These patients can then be monitored for cancer that evades or does not evade the immune system of the patients (thus, is likely or not likely to show better prognosis and/or better response to cancer therapies). Reference values for a biomarker associated with a cancer that is likely or not likely to evade the immune system of a subject (thus, is likely or not likely to show better prognosis and/or better response to cancer therapies) can then be determined based on the progression of cancer that evades or does not evade the immune systems of the patients whose samples were analyzed.

Additional examples of determining a reference value for a biomarker associated with a cancer that is likely or not likely to evade the immune system of a subject (thus, is likely or not likely to show better prognosis and/or better response to cancer therapies) are well-known to a person of ordinary skill in the art and such embodiments are within the purview of the invention.

Once a subject is identified as having a cancer that is likely or not likely to evade the immune system of the subject (thus, is likely or not likely to show better prognosis and/or better response to cancer therapies), the cancer in the subject is treated by administering an appropriate first therapy and/or second therapy to the subject. For example, for a subject identified as having a cancer that is likely to evade the immune system of the subject (thus, is likely to show poor prognosis and/or poor response to cancer therapies), the step of treating the cancer includes administering a first therapy and/or a second therapy to the subject, whereas to a subject identified as having a cancer that is not likely to evade the immune system of the subject (thus, is likely to show good prognosis and/or good response to cancer therapies), the step of treating the cancer includes administering a first therapy to the subject and/or withholding the administration of the second therapy.

According to the invention, a first therapy is a non-immunotherapeutic treatment or an immunotherapy designed to kill and/or control the proliferation of cancer cells, whereas a second therapy is designed to reduce the ability of the cancer cells to evade the immune system of the subject. In one embodiment, the second therapy is directed to activing the WIC class I transactivation pathway by increasing the expression and/or activity of NLRC5 protein in the cancer cells.

In one embodiment, a first therapy is a non-immunotherapeutic treatment designed to kill and/or control the proliferation of cancer cells.

In another embodiment, a first therapy is an immunotherapy. An immunotherapy against a cancer comprises using the subject's immune system to treat cancer, for example, use the subject's immune cells to kill and/or control the proliferation of the cancer cells.

Thus, in one embodiment, the immunotherapy comprises blocking the ability of the immune checkpoint proteins, i.e., the proteins that limit the strength and duration of immune responses. Blocking the activity of the immune checkpoint proteins increases the ability of the immune system to target cancer cells. An example of an immunotherapy that blocks the immune checkpoint proteins comprises administering a pharmaceutically effective amount of an anti-CTLA-4 antibody, for example, ipilimumab, to the subject. Ipilimumab is a monoclonal antibody against CTLA-4, a protein receptor that downregulates the immune system. CTLA4 is expressed on the surface of cytotoxic T lymphocytes to inactivate these T cells, thereby reducing the strength of immune responses. Ipilimumab binds to CTLA4 and prevents it from sending its inhibitory signal. In a specific embodiment, melanoma in a subject is treated with ipilimumab.

Another example of an immunotherapy that blocks the immune checkpoint proteins comprises administering to the subject a pharmaceutically effective amount of an anti-PD-1 antibody, for example, nivolumab or pembrolizumab. Nivolumab is a monoclonal antibody against PD-1, a protein that downregulates T-cell activation, thereby reducing the strength of immune responses. Nivolumab binds to and blocks the activation of PD-1. In a certain embodiment, unresectable or metastatic melanoma or squamous non-small cell lung cancer in a subject is treated with nivolumab.

Pembrolizumab is also a monoclonal antibody that targets PD-1. In a certain embodiment advanced melanoma in a subject who carries a BRAF mutation is treated with pembrolizumab. In a further embodiment metastatic non-small cell lung cancer in a subject whose tumors express PD-L1 is treated with pembrolizumab.

Atezolizumab is a monoclonal antibody that targets programmed death ligand-1 (PD-L1). Atezolizumab binds to PD-L1 expressed on tumor cells and tumor-infiltrating immune cells and blocks its interactions with PD-1 and B7.1 receptors. By inhibiting PD-L1, atezolizumab activates T cells. In one embodiment, a cancer in a subject is treated with atezolizumab.

In another embodiment of the invention, the immunotherapy comprises adoptive cell transfer (ACT). In one embodiment, ACT comprises isolating T cells from a subject that have infiltrated the subject's tumor. These T cells are called tumor-infiltrating lymphocytes (TIL). TILs showing the greatest recognition of the subject's cancer cells are selected and these cells are cultured and amplified in vitro. The cells are optionally activated by the treatment with cytokines and are administered to the subject.

In another embodiment, ACT comprises isolating a subject's T cells, and these T cells are genetically modified to express a chimeric antigen receptor (CAR). A CAR is a modified form of the T-cell receptor, which allows a T cell-expressing CAR to attach to specific proteins on the surface of cancer cells. Once bound to a cancer cell, the modified T cell becomes activated and attacks the cancer cell.

In a further embodiment, the immunotherapy comprises administering therapeutic antibodies to the subject. Therapeutic antibodies cause immune system-mediated destruction of cancer cells. A cancer cell having the antibody bound to it is recognized by certain immune cells or proteins, for example, a complement protein, which mediates cancer cell death, and is killed, for example, via antibody-dependent cell-mediated cytotoxicity or complement-dependent cytotoxicity.

In another embodiment, the therapeutic antibodies administered to a subject are conjugated with a drug in an “antibody-drug conjugate (ADC).” ADCs comprise antibodies or fragments of antibodies conjugated to a cancer drug. The antibody portion of the ADC binds to a target molecule on the surface of a cancer cell and delivers the cancer drug to the cancer cell. Once the cancer drug is taken up by the cell, the drug kills the cell.

In one embodiment, the ADC is ado-trastuzumab emtansine and is used to treat breast cancer in a subject. In another embodiment, the ADC is brentuximab vedotin and is used to treat Hodgkin's lymphoma and non-Hodgkin's T-cell lymphoma. In a further embodiment, the ADC is ibritumomab tiuxetan and used to treat non-Hodgkin's B-cell lymphoma.

In certain embodiments, the immunotherapy comprises administering a non-antibody immune system molecule conjugated to a cancer drug to a subject. For example, denileukin diftitox, which consists of interleukin-2 (IL-2) attached to diphtheria toxin, is administered to a subject to treat a cancer, for example, cutaneous T-cell lymphoma.

In a further embodiment, the immunotherapy comprises administering a cancer vaccine to a subject. A cancer vaccine is made up of cancer cells, parts of cells, or pure antigens. A cancer vaccine can also comprise a subject's immune cells that are exposed to cancer cells, parts of cells, or pure antigens. Cancer vaccines are designed to treat cancer in a subject by strengthening the subject's immune defenses against the cancer. In one embodiment, the cancer vaccine sipuleucel-T is administered to a subject to treat a cancer, for example, metastatic prostate cancer. Sipuleucel-T is a personalized treatment that works by programming a subject's immune system to kill cancer cells. Sipuleucel-T is prepared specifically for each subject.

In one embodiment, the immunotherapy comprises administering an immune system modulator to a subject. An immune system modulator is typically a protein that enhances a subject's immune response against cancer. Non-limiting examples of immune system modulators are cytokines, for example, interleukin and interferon, and growth factors. In one embodiment, interferon is administered to a subject that enhances the subject's immune response to cancer cells by activating natural killer cells and dendritic cells.

Additional immunotherapies are well-known to a person of ordinary skill in the art and such embodiments are within the purview of the invention.

As noted above, according to the invention, a second therapy is designed to reduce the ability of the cancer cells to evade the immune system of the subject. In one embodiment, the second therapy designed to activate the MHC class I transactivation pathway by increasing the expression of nlrc5 and/or the translation of NLRC5 mRNA and/or the activity of NLRC5 protein in the cancer cells.

In a certain embodiment, the second therapy induces the MHC class I antigen presentation pathway by activators of nlrc5, NLRC5 mRNA or NLRC5 protein. A number of approaches can be designed to induce the MHC class I antigen presentation pathway by activators of NLRC5. These approaches include:

a) activation of NLRC5 protein activity by agonists or activators, for example, small compounds, nucleotides, proteins or peptides;

b) introducing a wild-type or mutant NLRC5 protein or a nucleotide encoding the wild-type or mutant NLRC5 protein into the cancer cells;

c) inducing demethylation of genomic DNA by using non-specific demethylation agents; or

d) inducing demethylation of nlrc5 by using site-specific demethylation agents.

In an embodiment, the second therapy comprises administering a wild-type or a mutant NLRC5 protein or a nucleotide encoding the wild-type or mutant NLRC5 protein to the subject, particularly into the cancer cells. In one embodiment, the mutant NLRC5 protein has transcription factor activity higher than the wild-type NLRC5 protein.

The wild-type or mutant NLRC5 protein can be synthesized recombinantly. In an embodiment, the mutant NLRC5 contains one or more of the following mutations as compared to the wild-type NLRC5 protein: L181P, R262C, R550W, A737D, H1717fs*29, R1830C, and Q1847** as well as Walker B mutant NLRC5 (Meissner et al., 2010 PNAS, see Worldwide Website: ncbi.nlm.nih.gov/pubmed/20639463). These mutants are shown to have increased transcriptional activity and, therefore, provide suitable therapeutic agents to induce the MHC class I antigen presentation pathway (FIG. 3).

In another embodiment, a nucleotide encoding the wild-type or mutant NLRC5 protein is administered to a subject, particularly into the cancer cells.

Methods of recombinantly producing a protein are well-known to a person of ordinary skill in the art. Similarly, methods of producing a nucleotide encoding a protein of interest, for example, a plasmid or a viral vector, suitable for administration into a subject, particularly into the cancer cells of a subject, are also well-known to a person of ordinary skill in the art. Such embodiments are within the purview of the invention.

In one embodiment, a wild-type or a mutant NLRC5 protein or a nucleotide encoding the wild-type or a mutant NLRC5 protein is encapsulated in liposomes designed to deliver their contents into the cancer cells of a subject. In a further embodiment, the liposomes are modified in a manner that facilitates the delivery of their contents into the cancer cells of a subject. Techniques of modifying liposomes to facilitate the delivery of their contents into the cancer cells are described below.

In another embodiment, a wild-type or a mutant NLRC5 protein or a nucleotide encoding the wild-type or a mutant NLRC5 protein is conjugated to nanoparticles that are designed to deliver the protein or nucleotide into the cancer cells of a subject. Techniques of modifying nanoparticles to facilitate the delivery of the proteins or nucleotides into the cancer cells are also described below.

In one embodiment, the second therapy comprises administering to the subject an agent that induces demethylation of genomic DNA, preferably demethylation of nlrc5, more preferably demethylation of the nlrc5 promoter (SEQ ID NO: 4), even more preferably demethylation of a portion of the nlrc5 promoter having the sequence of SEQ ID NO: 5 or 6.

In an embodiment, the agent that induces demethylation of the genomic DNA comprises DNA methylation inhibitors, for example, inhibitors of enzymes that cause DNA methylation. Non-limiting examples of the DNA methylation inhibitors include: 4-amino-1-(2-deoxy-β-D-erythro-pentofuranosyl)-1,3,5-triazin-2(1H)-one (Decitabine); 4-amino-1-β-D-ribofuranosyl-1,3,5-triazin-2(1H)-one (5-azacytidine); 1-β-D-ribofuranosyl-2(1H)-pyrimidinone (Zebularine); N-phthalyl-L-tryptophan; 5-iodo-7-β-D-ribofuranosyl-7H-pyrrolo[2,3-d]pyrimidin-4-amine (5-iodotubercidin); 6-[(4-bromo-2-thienyl)methoxy]-9H-purin-2-amine (lomeguatrib); and N-[4-[(2-amino-6-methyl-4-pyrimidinyl)amino]phenyl]-4-(4-quinolinylamino)benzamide.

In another embodiment, the agent that induces demethylation of the genomic DNA is a demethylation inducer, for example, a DNA demethylase enzyme, for example, Tet Methylcytosine Dioxygenase 2. Accordingly, in one embodiment, a nucleotide that encodes a DNA demethylase enzyme is administered to the subject, particularly into the cancer cells.

A nucleotide encoding a DNA demethylase enzyme can be DNA or RNA. DNA encoding a DNA demethylase enzyme can be a plasmid or a viral vector. Additional DNA constructs suitable for the delivery of a nucleotide into cancer cells are well-known to a person of ordinary skill in the art and such embodiments are within the purview of the invention.

In a further embodiment, the second therapy comprises administering to the subject an agent that induces demethylation of nlrc5, preferably demethylation of the nlrc5 promoter having the sequence of SEQ ID NO: 4, even more preferably demethylation of a portion of the nlrc5 promoter having the sequence of SEQ ID NO: 5 or 6.

To induce target-specific demethylation, a sequence recognition module that specifically binds to nlrc5 or a portion of nlrc5 (SEQ ID NO: 4, 5 or 6) is fused to a DNA demethylase enzyme or a catalytic domain of a DNA demethylase enzyme. When the sequence recognition module binds to its target sequence, the demethylase enzyme demethylates the target DNA. Demethylation of nlrc5 or a portion thereof causes transcriptional activation of nlrc5 expression.

Non-limiting examples of sequence recognition modules include triple-helix forming oligonucleotides (TFOs); synthetic polyamides; DNA-binding domains of zinc finger proteins; transcription activator-like effectors (TALEs); and the Cas9 RNA-guided DNA binding proteins of the clustered regularly interspaced palindromic repeat (CRISPR) system.

In one embodiment, the nlrc5-specific demethylation is induced in the cancer cells of a subject based on the CRISPR/Cas9 gene regulation system. The CRISPR/Cas9 gene regulation system consists of a nuclease-null dCas9 protein fused to a demethylase enzyme, such as TET family proteins or demethylating agent. This fusion protein catalyzes demethylation at the target site. The fusion protein dCas9 fused to a demethylase enzyme causes demethylation of a target genomic locus via complementarity between an engineered guide RNA (gRNA) and the target site. As such, recruitment of the demethylase enzyme by dCas9 and a gRNA to the nlrc5 or a portion thereof modulates the methylation status of nlrc5 and activates nlrc5 expression.

Hilton et al. (2015) describe nuclease-null dCas9, genome-editing fusion proteins comprising dCas9 and the plasmids that encode for the genome-editing fusion proteins. The Hilton et al. reference is herein incorporated by reference in its entirety.

Based on the knowledge in the art, a person of ordinary skill in the art can design nuclease-null dCas9, fusion proteins comprising dCas9 and a DNA demethylase enzyme, an appropriate gRNA that targets the fusion protein to the nlrc5 or a portion thereof and plasmids that encode the fusion proteins comprising dCas9 and a DNA demethylase enzyme.

In another embodiment, targeted demethylation of nlrc5 or a portion thereof in the cancer cells of a subject is induced by a fusion of engineered TALE repeat arrays and a DNA demethylase enzyme, for example, the TET1 hydroxylase catalytic domain. The TALE-TET 1 fusion protein of the invention can be used to demethylate nlrc5 or a portion thereof to increase the expression of nlrc5. TALE repeat arrays can be engineered to bind nlrc5 or a portion thereof. Transcription activator-like effectors (TALEs) can be engineered to bind practically any desired DNA sequence including NLRC5 promoter. By engineering a TALE fused with demethylating enzyme or demethylating agents but not nuclease, NLRC5-promoter specific demethylation can be induced. Such TALE repeat arrays can be fused with full-length human TET1 or its catalytic domain.

Maeder et al. (2013) describe TALE-TET1 fusion proteins and the plasmids that encode these genome-editing fusion proteins. The Maeder et al. reference is herein incorporated by reference in its entirety. Based on the knowledge in the art, a person of ordinary skill in the art can design TALE-TET1 fusion proteins targeted to nlrc5 as described herein and the plasmids that encode for these genome-editing fusion proteins.

In another embodiment, nlrc5-specific demethylation is induced in the cancer cells of a subject using a DNA-binding domain (DBD) of zinc finger protein (ZFP)-based targeted genomic DNA demethylation. The DBD of ZFP is modified to specifically bind to the nlrc5 promoter or a portion thereof. ZFP chimera are chimeric proteins composed of DNA binding zinc finger domain and another domain. NLRC5 promoter specific ZFP recombinant protein can be developed by screening based on including but not restricted to bipartite selection, phage display, in vitro selection and zinc finger array. Such ZFP is fused to a DNA demethylase enzyme or demethylating agents, for example, the TET1 hydroxylase or its catalytic domain. The ZFP-TET1 fusion protein of the invention can be used to demethylate the nlrc5 promoter or a portion thereof to increase the expression of nlrc5 in the cancer cells of a subject.

Ji et al. (2014) describe ZFP-TET1 fusion proteins and the plasmids that encode for these genome-editing fusion proteins. The Ji et al. reference is herein incorporated by reference in its entirety. Based on the knowledge in the art, a person of ordinary skill in the art can design ZFP-TET1 fusion proteins targeted to nlrc5 or a portion thereof as described herein and the plasmids that encode for these genome-editing fusion proteins.

Thakore et al. (2015) describe various embodiments of genome-editing fusion proteins and the methods of using them for targeted editing of specific genomic sites. The Thakore et al. reference is incorporated herein in its entirety.

In another embodiment, nlrc5-specific demethylation is induced in the cancer cells of a subject using DNA or RNA nucleotides including but not limited to TFO (triplex-forming oligonucleotides) based targeted genomic DNA demethylation. TFOs are single polynucleotide strands that bind to their target sequence in the major groove of double-stranded DNA. When fused to a DNA demethylase enzyme, TFOs can be used to direct the DNA demethylase enzyme to nlrc5 or a portion thereof. van der Gun et al. (2010) describe certain embodiments of TFOs and the methods of using them for target editing of specific genomic sites. The van der Gun et al. reference is incorporated herein in its entirety. Based on the knowledge in the art, a person of ordinary skill in the art can design TFO fused to DNA demethylase or DNA methylating agents targeted to nlrc5 or a portion thereof as described herein.

In a further embodiment, the nlrc5-specific demethylation is induced in the cancer cells of a subject using synthetic polyamide-based targeted genomic DNA demethylation. Synthetic polyamides comprise two anti-parallel polyamide stretches consisting of hydroxypyrrole (Hp), imidazol (Im) and pyrrol (Py), which build a hairpin formation through side-by-side amino acid pairing. The hairpin structure binds to specific base pairs in the minor groove of double helical DNA by hydrogen bonding. NLRC5 specific demethylation is provided by synthetic polyamines developed by screening for NLRC5 promoter sequence, conjugated with demethylating enzymes or demethylating agents.

Hochhauser et al. (2007) describe certain embodiments of synthetic polyamides and the methods of using them for target editing of specific genomic sites. The Hochhauser et al. reference is incorporated herein in its entirety. Based on the knowledge in the art, a person of ordinary skill in the art can design synthetic polyamides fused to DNA demethylase targeted to nlrc5 or a portion thereof as described herein.

A plasmid that encodes the dCas9-DNA demethylase fusion/gRNA system, TALE-TET1 fusion protein or ZFP-TET1 fusion protein can be delivered specifically to the cancer cells of a subject. Similarly, the TFO or polyamide fused to a DNA demethylase can be delivered specifically to the cancer cells of a subject. In one embodiment, the plasmid or the TFO or polyamide fused to a DNA demethylase is encapsulated in liposomes designed to deliver their contents into the cancer cells of a subject. Similarly, the plasmids, fusion proteins, TFO or polyamide fused to a DNA demethylase can be conjugated to nanoparticles, particularly nanoparticles that are designed to deliver these agents specifically to cancer cells.

The liposomes containing the proteins or nucleotides of the invention can be modified to contain binding agents, for example, binding proteins, antibodies or fragments of antibodies, that specifically bind to biomolecules, for example, cell surface receptors, that are present on the surface of the cancer cells or that are present on the surface of the cancer cells at a higher level compared to non-target cells. Certain examples of using specific cell-surface biomolecules present on the surface of cancer cells and corresponding binding agents that can be incorporated in liposomes are described in Deshpande et al., the contents of which are herein incorporated by reference in their entirety. Additional examples of cell-surface biomolecules specifically present or overexpressed on the surface of cancer cells are known to a person of ordinary skill in the art and such embodiments are within the purview of the claimed invention. Liposomes can be administered topically, orally, or via pulmonary or parenteral routes.

Various liposome compositions are known to a person of ordinary skill in the art. For example, Maherani et al. (2011) describe manufacturing techniques for liposomes, compositions of liposomes, methods of encapsulating biomolecules into liposomes, and methods of producing pharmaceutical compositions comprising liposomes. The Maherani et al. reference is herein incorporated by reference in its entirety.

In one embodiment, the liposomes contain agents that destabilize the liposome membrane and cause the release of contents in the aqueous compartment into the target cells.

The destabilizing agents can destabilize the liposomes in response to a lower pH, for example, the lower pH present in the endosomes/lysosome compartments of the target cells. In certain embodiments, temperature-sensitive or radiation-sensitive destabilizers are used where the cancer cells can be subjected to conditions that cause release of the contents of the liposomes into the cancer cells.

The nanoparticles designed to deliver the agents of the invention specifically into the cancer cells can be conjugated to binding agents, for example, binding proteins, antibodies or fragments of antibodies, which specifically bind to biomolecules, for example, cell surface receptors, that are present on the surface of the cancer cells or that are present on the surface of the cancer cells at a higher level compared to non-target cells. Certain examples of specific cell-surface biomolecules present on the surface of the cancer cells and corresponding binding agents are described in Deshpande et al. and can be implemented in the invention. Additional examples of cell-surface biomolecules specifically present or overexpressed on the surface of the cancer cells are known to a person of ordinary skill in the art and such embodiments are within the purview of the claimed invention.

Based on an appropriate selection of a first therapy and a second therapy, the invention provides various treatment regimens for a subject identified as having a cancer that is likely or not likely to evade the immune system of the subject.

In one embodiment, a first therapy is a non-immunotherapeutic treatment and a second therapy is not administered to a subject identified as having a cancer that is likely or not likely to evade the immune system of the subject. As the first therapy does not utilize the subject's immune system, the ability of the subject's cancer to evade the immune system has less effect on the success of the first therapy.

In another embodiment, the first therapy is a non-immunotherapeutic treatment and the second therapy is not administered to a subject identified as having a cancer that is not likely to evade the immune system of the subject. As the subject's cancer is not likely to evade the subject's immune system, a second therapy, which is designed to reduce the ability of the cancer cells to evade the immune system of the subject, may not be required. Also, withholding the second therapy from a subject having a cancer that is not likely to evade the immune system of the subject avoids the harmful side effects that may be caused by the second therapy.

In a further embodiment of the invention, the first therapy is a non-immunotherapeutic treatment and the second therapy is administered to a subject identified as having a cancer that is likely to evade the immune system of the subject. The second therapy is designed to reduce the ability of the subject's cancer to evade the immune system. Therefore, the subject's immune system may act synergistically with the first therapy and the second therapy to provide therapeutic benefit to the subject. As such, administering a second therapy to a subject having a cancer that is likely to evade the immune system of the subject may increase the success of the first therapy.

In a particular embodiment, the first therapy is an immunotherapy and the second therapy is not administered to a subject identified as having a cancer that is not likely to evade the immune system of the subject. As the subject's cancer is not likely to evade the subject's immune system, the second therapy, which is designed to reduce the ability of the cancer cells to evade the immune system of the subject, may not be required. Also, withholding the second therapy from a subject having a cancer that is not likely to evade the immune system of the subject avoids the harmful side effects that may be caused by the second therapy.

In a further embodiment of the invention, the first therapy is an immunotherapy and the second therapy is administered to a subject identified as having a cancer that is likely to evade the immune system of the subject. The second therapy is designed to reduce the ability of the subject's cancer to evade the immune system. Therefore, the subject's immune system acts with the first therapy and the second therapy to kill the cancer cells. Therefore, administering a second therapy to a subject having a cancer that is likely to evade the immune system of the subject increases the success of the first therapy.

Additional combinations of the first therapy and/or the second therapy can be designed by a person of ordinary skill in the art based on the invention and such embodiments are within the purview of the invention. The first and the second therapies can be administered simultaneously or separately to a subject. In an embodiment, the first therapy is administered before or after the second therapy. In a further embodiment, the first therapy and/or the second therapy are administered in a sub-therapeutic amount. When at least the first and/or the second therapy are administered in a sub-therapeutic amount, a synergistic effect between these therapies provides a therapeutic benefit to a subject.

### Materials and Methods

Data Sets

Tumor types were selected based on availability of the gene-level RNA-seq expression data from The Cancer Genome Atlas (TCGA). 21 solid tumor types were analyzed including adrenocortical carcinoma (ACC), bladder urothelial carcinoma (BLCA), breast invasive carcinoma (BRCA), cervical squamous cell carcinoma and endocervical adenocarcinoma (CESC), colon adenocarcinoma (COAD), glioblastoma multiforme (GBM), head and neck squamous cell carcinoma (HNSC), kidney chromophobe (KICH), kidney renal clear cell carcinoma (KIRC), kidney renal papillary cell carcinoma (KIRP), brain lower grade glioma (LGG), liver hepatocellular carcinoma (LIHC), lung adenocarcinoma (LUAD), lung squamous cell carcinoma (LUSC), ovarian serous cystadenocarcinoma (OV), prostate adenocarcinoma (PRAD), skin cutaneous melanoma (SKCM), rectum adenocarcinoma (READ), thyroid carcinoma (THCA), uterine corpus endometrial carcinoma (UCEC) and uterine carcinosarcoma (UCS). GBM and LGG are defined as a single cancer type (GBMLGG). The abbreviations in this study and the number of samples in each analysis are shown in Table 1 and Table 2, respectively.

TABLE 1
The abbreviations of the samples from
The Cancer Genome Atlas (TCGA).

| TCGA | | Abbreviation in |
|---|---|---|
| ID | Tumor type | this study |
| ACC | Adrenocortical carcinoma | Adrenal |
| BLCA | Bladder urothelial carcinoma | Bladder |
| BRCA | Breast invasive carcinoma | Breast |
| CESC | Cervical squamous cell carcinoma | Cervical |
| | and endocervical adenocarcinoma | |
| COAD | Colon adenocarcinoma | Colon |
| GBM/ | Glioblastoma multiforme/ | Glioma/ |
| LGG | Brain lower grade glioma | Glioblastoma |
| HNSC | Head and neck squamous cell | Head/neck |
| | carcinoma | |
| KICH | Kidney chromophobe | Kidney chromophobe |
| KIRC | Kidney renal clear cell carcinoma | Kidney clear cell |
| KIRP | Kidney renal papillary cell carcinoma | Kidney papillary |
| LIHC | Liver hepatocellular carcinoma | Hepatocellular |
| LUAD | Lung adenocarcinoma | Lung adeno |
| LUSC | Lung squamous cell carcinoma | Lung squamous |
| OV | Ovarian serous cystadenocarcinoma | Ovarian |
| PRAD | Prostate adenocarcinoma | Prostate |
| READ | Rectum adenocarcinoma | Rectal |
| SKCM | Skin cutaneous melanoma | Melanoma |
| THCA | Thyroid carcinoma | Thyroid |
| UCEC | Uterine corpus endometrial carcinoma | Uterine ca. |
| UCS | Uterine carcinosarcoma | Uterine sa. |

TABLE 2
The number of samples used in the analysis.

| | | | Copy number | | Mutation | | | |
|---|---|---|---|---|---|---|---|---|
| | Correlation | Methylation | analysis | | analysis | | Kaplan-meter analysis | |
| Tumor type | analysis | analysis | Diploid | CN loss | Wild type | Mutant | NLRCS high | NLRCS low |
| Adrenal | 79 | 79 | 26 | 5 | 76 | 3 | 19 | 20 |
| Bladder | 408 | 408 | 211 | 122 | 402 | 6 | 46 | 47 |
| Breast | 1100 | 775 | 311 | 644 | 1034 | 16 | 154 | 155 |
| Cervical | 308 | 308 | 201 | 58 | 301 | 7 | 72 | 72 |
| Colon | 283 | 274 | 195 | 23 | 262 | 27 | 67 | 68 |
| Giloma/Glioblastoma | 895 | 588 | 578 | 57 | 893 | 1 | 125 | 125 |
| Head/neck | 522 | 522 | 308 | 92 | 516 | 6 | 101 | 101 |
| Kidney chromophobe | 66 | 55 | 40 | 6 | 65 | 1 | 14 | 14 |
| Kidney clear cell | 534 | 318 | 405 | 22 | 526 | 6 | 126 | 126 |
| Kidney papillary | 283 | 257 | 128 | 7 | 252 | 1 | 37 | 37 |
| Hepatocellular | 372 | 371 | 199 | 146 | 370 | 2 | 32 | 33 |
| Lung adeno | 517 | 450 | 257 | 146 | 501 | 16 | 105 | 107 |
| Lung squamous | 502 | 370 | 224 | 164 | 497 | 5 | 96 | 96 |
| Ovarian | 265 | — | 103 | 353 | 250 | 5 | 55 | 55 |
| Prostate | 498 | 498 | 361 | 122 | 494 | 4 | 61 | 62 |
| Rectal | 99 | 93 | 63 | 9 | 99 | 0 | 24 | 24 |
| Melanoma | 471 | 468 | 210 | 116 | 438 | 33 | 85 | 85 |
| Thyroid | 509 | 502 | 454 | 2 | 509 | 0 | 102 | 103 |
| Uterine ca. | 177 | 158 | 155 | 58 | 175 | 2 | 41 | 40 |
| Uterine sa. | 57 | — | 11 | 37 | 56 | 1 | 14 | 14 |

Gene Expression Analysis

TCGA gene expression data (Illumina HiSeq 2000 RNA Sequencing Version 2 analysis) were accessed through GDAC Firehose (see: gdac.broadinstitute.org). The expression of nlrc5 and MHC class I-related genes (hla-a, hla-b, hla-c, b2m, psmb9 (lmp2), psmb8 (lbp7), and tap1) were rescaled to Transcripts Per Million (TPM) calculation of “scaled_estimate” which shows tau value multiplied by 10⁶. Spearman's rank correlation test was used to assess the correlation between the expression of nlrc5 and MHC class I related genes in biopsy samples of 20 cancer types from 7747 cancer patients as well as cytolytic activity in a total of 7749 cancer patients. Cytolytic activity was shown via analysis of two key genes of cytolytic T cells: granzyme A (gzma) and perforin (prf1).

To assess the correlation between the expression of nlrc5 and cd8a expression in a total of 20 cancer types from 6277 patients or ncam1 (cd56) gene expression in 19 cancer types excluding glioma/glioblastoma from 5685 patients, Spearman's rank correlation test was performed.

Methylation Analysis

DNA methylation data (Illumina Infinium Human DNA Methylation 450) for 18 TCGA tumor types were accessed through GDAC Firehose. DNA methylation levels were measured by the probe specific to the CpG island in the nlrc5 promoter (cg16411857, FIG. 2B, SEQ ID NO: 6) and shown by beta values ranging from 0 to 1, with 0 corresponding to the minimal level of DNA methylation and 1 to the maximal level of DNA methylation. A methylation level with a beta value less than 0.3 was defined as unmethylated. Spearman's rank correlation test was used to assess the correlation between DNA methylation levels in the nlrc5 promoter and the expression level of nlrc5 or MHC class I-related genes in a total of 6523 TCGA tumor samples as well as gzma and prf1 in a total of 6528 cancer patients. Also, Spearman's rank correlation test was performed to assess the correlation between DNA methylation levels in the nlrc5 promoter and cd8a gene expression in 18 cancer types from 6277 patients or ncam1 (cd56) gene expression in 17 cancer types excluding glioma/glioblastoma from 5685 patients. DNA methylation levels of the ciita were measured by the probe specific to the promoter of ciita gene (cg08985333) and shown by beta values. Spearman's rank correlation test was used to assess the correlation between DNA methylation levels in the ciita promoter and the expression level of the hla-b gene in a total of 5667 TCGA tumor samples. DNA methylation levels of MHC class I genes were measured by probes specific to hla-a (cg23489273), hla-b (cg00241218), hla-c (cg16097079), b2m (cg08350173), lmp2 (cg03778035), lmp7 (cg05545172) and tap1 (cg14530528).

Copy Number Analysis

TCGA copy number data (Affymetrix Genome-Wide Human SNP Array 6.0) generated by the GISTIC2 algorithm were accessed through cBioPortal. Values were shown by −2=homozygous deletion; −1=hemizygous deletion; 0=neutral/no change; 1=gain; 2=high level amplification. −2 and −1 indicate copy number loss and 0 indicate diploid. For total of 7730 samples consisting of 20 tumor types, the percentage of cancer patients who carried nlrc5 copy number (CN) loss was determined and the expression level of nlrc5 and MHC class I-related genes was compared between diploid and copy number loss group. Statistical p-values were determined by Mann-Whitney test. The nlrc5 and HLA-B gene expression levels were visualized by heatmaps generated by GENE-E (see, Worldwide Website: broadinstitute. org/cancer/software/GENE-E/).

Mutation Analysis

For representing mutation sites in nlrc5, somatic mutation data were accessed through the cBioPortal and the Catalogue of Somatic Mutations in Cancer (COSMIC, see: Worldwide Website: cancer.sanger.ac.uk/cosmic). To evaluate the expression level of MHC class I-related genes between nlrc5 wild-type and mutant groups, data sets comprised of both gene expression and mutation profiles were selected from the TCGA database (Illumina Genome Analyzer DNA Sequencing). The percentage of each mutation was estimated over the total number of mutations for 20 TCGA tumor types, consisting of 7752 samples. The mutation types include missense, silent, nonsense, frameshift insertion, in-frame deletion, frameshift deletion and splice mutations. Samples carrying multiple mutations were shown as complex. Also, mutation rates for all samples and each tumor type were calculated.

Furthermore, the correlation analysis between the expression levels of MHC class I-related genes and nlrc5 mutation status were assessed. In order to evaluate the ability of wild-type and mutant nlrc5 to induce the expression of MHC class I-related genes in each patient, MHC class I gene levels were normalized by nlrc5 expression levels. Every mutation type excluding silent mutations was treated equally and the patients who have multiple mutations were treated as one event. The patients who have silent mutations were treated as a wild-type group.

Survival Analysis

Clinical data were accessed through GDAC Firehose (Supplementary Data 5). 20 TCGA tumor types of 5554 patients were stratified by the expression levels of nlrc5, MHC class I and related genes and DNA methylation levels in the nlrc5 promoter and other MHC class I and related genes. The top and bottom quartiles (expression of nlrc5/MHC class I and related genes/markers for cytotoxic CD8⁺ T cell activity or methylation level of nlrc5/MHC class I and related genes high and low, respectively) were used for analysis. Overall survival time was measured from the date of diagnosis to the date of death or the last follow-up. Five-year survival rates were compared between the groups and the statistical significance was determined by chi-square test. Survival outcomes were estimated according to the Kaplan-Meier method and compared between groups by log-rank test and Gehan-Breslow-Wilcoxon test. The log-rank statistic assesses group differences equally across the full observation time, whereas the Wilcoxon statistic weights the early events.

Expression Vectors

The expression vector for human gfp-nlrc5 in pcDNA3.1 was previously described. The reporter gene construct used for MHC class I promoter activity (HLA-B250) was also previously described. Selected nlrc5 mutants were constructed using pcDNA3.1-gfp-nlrc5 by site-directed mutagenesis using the primers listed in Table 3. All the plasmids were confirmed by sequencing (Gene Technologies Laboratory).

TABLE 3
List of primers used for construction of selected nlrc5 mutant
expression vectors, related to experimental procedures.

| | | Forward | SEQ | Reverse | SEQ |
|---|---|---|---|---|---|
| AA | Mutation | Primer | ID | Primer | ID |
| position | (CDS) | Sequence | NO: | Sequence | NO: |
| 181 | c.542T > C | TCCAATCCCG | 7 | CTGTGGCCCGGCGCGG | 8 |
| | | CGCCGGGCCA | | GATTGGA | |
| 262 | c.784C > T | GTTCCTTTTT | 9 | GTTGAGCTGGCAGAAT | 10 |
| | | GAATTCTGCC | | TCAAAAAGGAACAGGG | |
| | | AGCTCAACTT | | C | |
| | | G | | | |
| 386 | c.1156C > T | GCCATCGTGG | 11 | CCCCTCCCACGATGGCT | 12 |
| | | GAGGGGGCC | | GGG | |
| 550 | c.1648C > T | GCTGGGTACA | 13 | GCTTTGGTCCACTGTAC | 14 |
| | | GTGGACCAA | | CCAGCGGG | |
| | | AGCTAGA | | | |
| 496 | c.1487C > T | GCCTGCTGAC | 15 | CAGACGCAGAAGAAAG | 16 |
| | | TTTCTTCTGC | | TCAGCAGGCTGT | |
| | | GTCTGCAC | | | |
| 574 | c.1721G > A | GCACCTGCCA | 17 | GGAAGGGGTGGCAGGT | 18 |
| | | CCCCTTCCTT | | GCAGGA | |
| | | AGC | | | |
| 737 | c.2210C > A | CACCTGGTGA | 19 | CAGAGAGGCAAATCTT | 20 |
| | | AAGATTTGCC | | TCACCAGGTGGC | |
| | | TCTCTGTCC | | | |
| 884 | c.2650G > A | GAACCAGCTG | 21 | GACAGCCTTCATTTTCC | 22 |
| | | GAAAATGAA | | AGCTGGTTCCC | |
| | | GGCTGTCGG | | | |
| 1005 | c.3013G > A | GCTGCCACCT | 23 | GTGGAGGTGACTGAGG | 24 |
| | | CAGTCACCTC | | TGGCAGCTTC | |
| | | CACCTC | | | |
| 1173 | c.3518C > T | GCAGCTGAGC | 25 | GTCCCATCTGGCTCAGC | 26 |
| | | CAGATGGGA | | TGCAGC | |
| | | CTGTC | | | |
| 1512 | c.4534G > A | GAGGGCCTCA | 27 | CCAGGTGGGTGAGGCC | 28 |
| | | CCCACCTGGC | | CTCGG | |
| | | A | | | |
| 1717 | c.5144delC | CCCAGGCCCT | 29 | CAA | 30 |
| | | GGATGGATCC | | ATGGGGGATCCATCCA | |
| | | CCCAT | | GGGCCT | |
| 1830 | c.5488C > T | GCATCCAAGT | 31 | GGTTATTCCAGAGGCA | 32 |
| | | CATCTGCCTC | | GATGACTTGGATGCTA | |
| | | TGGAATAACC | | G | |
| | | CC | | | |
| 1847 | c.5539C > T | CCTGAAGAGC | 33 | CTGGGCTCCTAGCTCTT | 34 |
| | | TAGGAGCCCA | | CAGGTGC | |
| | | GGCT | | | |

5-Azacytidine Treatment for DNA Demethylation

Cell lines were cultured in IMDM. All media were supplemented with 10% heat inactivated fetal bovine serum, 50 U/mL penicillin, 50 U/mL streptomycin, 4 mM L-glutamine and 10 mM HEPES. Cell lines were treated with 5-Azacytidine (3 μM) for DNA demethylation. RNA isolation and quantitative PCR were performed.

Cell Culture and Luciferase Assay

HEK293T cells were cultured in DMEM supplemented with 10% FBS and penicillin/streptomycin (Life Technologies) at 37° C. with 5% CO₂. Cells were transiently transfected using polyethylenimine at a DNA/polyethylenimine ratio of 1:3 in serum-free media. For luciferase assay, HEK293T cells were split at a density of 5×10⁴ cells/well into 24-well plates 1 day prior to transfection. Cells were co-transfected using 100 ng of gfp-nlrc5 or 100 ng of the specified nlrc5 mutant plasmids along with 100 ng of HLA-B250 luciferase reporter construct. 20 ng promoterless Renilla luciferase vector (pRL-null; Promega) were included for normalization of transfection efficiency. Cells were harvested 48 h post-transfection, and cell lysates were analyzed using the Dual-Luciferase Reporter Assay System (Promega) according to the manufacturer's instructions.

Statistical Analysis

Statistical analysis was performed using Graph Pad Prism software (GraphPad, San Diego, Calif., USA). All tests were two-sided, and p-value of less than 0.05 was considered statistically significant.

The subject application also provides the following non-limiting embodiments:

1. A method of identifying a subject as having a cancer that is likely or not likely to evade the immune system of the subject and, optionally, treating the subject, the method comprising the steps of:

(a) determining the amount of NLRC5 mRNA or NLRC5 protein and, optionally, neoantigen load, mutation number, and/or expression of genes involved in immune responses, including but not limited to CTLA4, PD1, PD-L1 and PD-L2 in:
i) a test sample obtained from the subject, and
ii) optionally, a control sample; and

(b) optionally, obtaining one or more reference values for the amount of NLRC5 mRNA or NLRC5 protein and, optionally, neoantigen load, mutation number, and/or expression of genes involved in immune responses, including but not limited to CTLA4, PD1, PD-L1 and PD-L2, and
(i) identifying the subject as having a cancer that is likely to evade the immune system of the subject based on the amount of NLRC5 mRNA or NLRC5 protein and, optionally neoantigen load, mutation number, and/or expression of genes involved in immune responses, including but not limited to CTLA4, PD1, PD-L1 and PD-L2, if determined, in a subject is lower in the test sample as compared to the control sample or the reference value and administering a first therapy and/or a second therapy to the subject to treat the cancer, or
(ii) identifying the subject as having a cancer that is not likely to evade the immune system of the subject based on the amount of NLRC5 mRNA or NLRC5 protein and, optionally, neoantigen load, mutation number, and/or expression of genes involved in immune responses, including but not limited to CTLA4, PD1, PD-L1 and PD-L2, if determined, in a subject is higher in the test sample as compared to the control sample or the reference value and administering a first therapy to the subject to treat the cancer and/or withholding the administration of the second therapy to the subject.

2. The method of embodiment 1, wherein the step of identifying the subject as having a cancer that is likely or not likely to evade the immune system of the subject comprises:
i) identifying the subject as having a cancer that is likely to evade the immune system of the subject if the amount of NLRC5 mRNA or NLRC5 protein and, optionally, neoantigen load, mutation number, and/or expression of genes involved in immune responses, including but not limited to CTLA4, PD1, PD-L1 and PD-L2 in a subject, if determined, in the test sample is lower than the amount of NLRC5 mRNA or NLRC5 protein and neoantigen load, mutation number, and/or expression of genes involved in immune responses, including but not limited to CTLA4, PD1, PD-L1 and PD-L2 in a subject, if determined, in the control sample or reference value, or
ii) identifying the subject as having a cancer that is not likely to evade the immune system of the subject if the amount of NLRC5 mRNA or NLRC5 protein and, optionally, neoantigen load, mutation number, or expression of genes involved in immune responses, including but not limited to CTLA4, PD1, PD-L1 and PD-L2 in a subject with higher levels of those parameters in the test sample, if determined, is equal to or higher than the amount of NLRC5 mRNA or NLRC5 protein and those parameters, if determined, in the control sample or reference value.

3. A method of identifying a subject as having a cancer that is likely or not likely to evade the immune system of the subject and, optionally, treating the subject, the method comprising the steps of:

(a) determining the transcription factor activity of NLRC5 protein and, optionally, neoantigen load, mutation number, and/or expression of genes involved in immune responses, including but not limited to CTLA4, PD1, PD-L1 and PD-L2 in a subject in:
i) a test sample obtained from the subject, and
ii) optionally, a control sample; and

(b) optionally, obtaining one or more reference values for the transcription factor activity of NLRC5 protein and, optionally, neoantigen load, mutation number, and/or expression of genes involved in immune responses, including but not limited to CTLA4, PD1, PD-L1 and PD-L2, and
(i) identifying the subject as having a cancer that is likely to evade the immune system of the subject based on the transcription factor activity of NLRC5 protein and, optionally, neoantigen load, mutation number, and/or expression of genes involved in immune responses, including but not limited to CTLA4, PD1, PD-L1 and PD-L2, if determined, in the test sample as compared to the control sample or the reference value and administering a first therapy and/or a second therapy to the subject to treat the cancer, or
(ii) identifying the subject as having a cancer that is not likely to evade the immune system of the subject based on the transcription factor activity of NLRC5 protein and, optionally, neoantigen load, mutation number, and/or expression of genes involved in immune responses, including but not limited to CTLA4, PD1, PD-L1 and PD-L2 if determined, in the test sample as compared to the control sample or the reference value and administering a first therapy to the subject to treat the cancer and/or withholding the administration of the second therapy to the subject.

4. The method of embodiment 3, wherein the step of identifying the subject as having a cancer that is likely or not likely to evade the immune system comprises:
i) identifying the subject as having a cancer that is likely to evade the immune system of the subject if the transcription factor activity of NLRC5 protein and the level of neoantigen load, mutation number, and/or expression of genes involved in immune responses, including but not limited to CTLA4, PD1, PD-L1 and PD-L2, if determined, in the test sample is lower than the transcription factor activity of NLRC5 protein or neoantigen load, mutation number, and/or expression of genes involved in immune responses, including but not limited to CTLA4, PD1, PD-L1 and PD-L2 in a subject in the control sample or reference value, if determined, or
ii) identifying the subject as having a cancer that is not likely to evade the immune system of the subject if the transcription factor activity of NLRC5 protein and, optionally, neoantigen load, mutation number, and/or expression of genes involved in immune responses, including but not limited to CTLA4, PD1, PD-L1 and PD-L2, if determined, in the test sample is equal to or higher than the transcription factor activity of NLRC5 protein and those parameters, if determined, in the control sample or reference value.

5. A method of identifying a subject as having a cancer that is likely or not likely to evade the immune system of the subject and, optionally, treating the subject, the method comprising the steps of:

(a) determining the sequence of the protein coding region of nlrc5 or a portion thereof and, optionally, neoantigen load, mutation number, and/or expression of genes involved in immune responses, including but not limited to CTLA4, PD1, PD-L1 and PD-L2 in a test sample obtained from the subject; and

(b) optionally, determining the sequence of NLRC5 protein encoded by nlrc5 or a portion thereof in the test sample and, optionally, determining the activity of a wild-type NLRC5 protein and the NLRC5 protein encoded by the nlrc5 in the test sample, and
(i) identifying the subject as having a cancer that is likely to evade the immune system of the subject if the NLRC5 protein in the test sample contains a mutation that reduces the transcription factor activity of NLRC5 protein and, if determined, neoantigen load, mutation number, and/or low expression of genes involved in immune responses, including but not limited to CTLA4, PD1, PD-L1 and PD-L2 is reduced in the test sample as compared to the wild-type NLRC5 protein and those parameters, if tested and, optionally, administering a first therapy and/or a second therapy to the subject to treat the cancer, or
(ii) identifying the subject as having a cancer that is not likely to evade the immune system of the subject if the NLRC5 protein in the test sample does not contain a mutation or contains a mutation that does not affect or increases the transcription factor activity of NLRC5 protein and neoantigen load, mutation number, and/or expression of genes involved in immune responses, including but not limited to CTLA4, PD1, PD-L1 and PD-L2, if determined, is elevated in the test sample as compared to the wild-type NLRC5 protein and control and/or reference values of those variables and, optionally, administering a first therapy to the subject to treat the cancer and/or withholding the administration of the second therapy to the subject.

6. The method of embodiment 5, wherein a subject having one or more of the following mutations in the NLRC5 protein as compared to the wild-type NLRC5 protein is identified as having a cancer that is likely to evade the immune system of the subject: L181P, R262C, R550W, A737D, H1717fs*29, R1830C, and Q1847*.

7. The method of embodiment 5, wherein a subject having one or more of the following mutations in the NLRC5 protein as compared to the wild-type NLRC5 protein is identified as having a cancer that is not likely to evade the immune system of the subject: R386W, S496F, R574H, D884N, T1173M, and A1512T.

8. A method of identifying a subject as having a cancer that is likely or not likely to evade the immune system of the subject, and treating the subject, the method comprising the steps of:

(a) determining the level of methylation of nlrc5 or a portion thereof and, optionally, neoantigen load, mutation number, and/or expression of genes involved in immune responses, including but not limited to CTLA4, PD1, PD-L1 and PD-L2 in a subject in:
i) a test sample obtained from the subject, and
ii) optionally, a control sample; and

(b) optionally, obtaining one or more reference values for the levels of methylation of nlrc5 or a portion thereof and, optionally, neoantigen load, mutation number, or expression of genes involved in immune responses, including but not limited to CTLA4, PD1, PD-L1 and PD-L2 in a subject, and
(i) identifying the subject as having a cancer that is likely to evade the immune system of the subject based on the level of methylation of nlrc5 or a portion thereof in the test sample as compared to the control sample or the reference value and neoantigen load, mutation number, and/or expression of genes involved in immune responses, including but not limited to CTLA4, PD1, PD-L1 and PD-L2, if determined, in a subject is lower, in comparison to a control sample or reference value, and optionally, administering a first therapy and/or a second therapy to the subject to treat the cancer, or
(ii) identifying the subject as having a cancer that is not likely to evade the immune system of the subject based on the level of methylation of nlrc5 or a portion thereof and neoantigen load, mutation number, and/or expression of genes involved in immune responses, including but not limited to CTLA4, PD1, PD-L1 and PD-L2 in a subject, if determined in the test sample as compared to the control sample or the reference value and, optionally, administering a first therapy to the subject to treat the cancer and/or withholding the administration of the second therapy to the subject.

9. The method of embodiment 8, wherein the step of identifying the subject as having a cancer that is likely or not likely to evade the immune system comprises:
i) identifying the subject as having a cancer that is likely to evade the immune system of the subject if the level of methylation of nlrc5 or a portion thereof in the test sample is higher than the level of methylation of nlrc5 or a portion thereof in the control sample and neoantigen load, mutation number, and/or expression of genes involved in immune responses, including but not limited to CTLA4, PD1, PD-L1 and PD-L2 in a subject, if determined are lower in the test sample in comparison to a control sample or reference value, or
ii) identifying the subject as having a cancer that is not likely to evade the immune system of the subject if the level of methylation of nlrc5 or a portion thereof in the test sample is equal to or lower than the level methylation of nlrc5 or a portion thereof in the control sample and neoantigen load, mutation number, and/or expression of genes involved in immune responses, including but not limited to CTLA4, PD1, PD-L1 and PD-L2 in a subject, if determined, are elevated in comparison to a test sample or reference value.

10. A method of identifying a subject as having a cancer that is likely or not likely to evade the immune system of the subject, and treating the subject, the method comprising the steps of:

(a) determining the beta-value for methylation of nlrc5 or a portion thereof and, optionally, neoantigen load, mutation number, and/or expression of genes involved in immune responses, including but not limited to CTLA4, PD1, PD-L1 and PD-L2 in a test sample obtained from the subject, and

(b) identifying the subject as:
i) having a cancer that is likely to evade the immune system of the subject if the beta-value for methylation of nlrc5 or a portion thereof in the test sample is above 0.2, 0.3 or 0.4, and neoantigen load, mutation number, and/or expression of genes involved in immune responses, including but not limited to CTLA4, PD1, PD-L1 and PD-L2 in a subject, if determined, is low in comparison to the control sample or reference value, or
b) having a cancer that is not likely to evade the immune system of the subject if the beta-value for methylation of nlrc5 or a portion thereof in the test sample is below 0.2, 0.3 or 0.4 and neoantigen load, mutation number, and/or expression of genes involved in immune responses, including but not limited to CTLA4, PD1, PD-L1 and PD-L2, if determined, is elevated in comparison to the control sample or reference value, if determined.

11. The method of embodiment 8, wherein the portion of nlrc5 has the sequence of SEQ ID NO: 4, 5 or 6.

12. A method of identifying a subject as having a cancer that is likely or not likely to evade the immune system of the subject, and, optionally, treating the subject, the method comprising the steps of:

(a) determining the copy number of nlrc5 in a test sample obtained from the subject, and
(i) identifying the subject as having a cancer that is likely to evade the immune system of the subject based on the copy number for nlrc5 being below about two in the test sample and, optionally, low neoantigen load, mutation number, and/or expression of genes involved in immune responses, including but not limited to CTLA4, PD1, PD-L1 and PD-L2 in a subject in comparison to a control sample or reference value administering a first therapy and/or a second therapy to the subject to treat the cancer, or
(ii) identifying the subject as having a cancer that is not likely to evade the immune system of the subject based on the copy number for nlrc5 being above about two in the test sample and optionally, elevated neoantigen load, mutation number, and/or expression of genes involved in immune responses, including but not limited to CTLA4, PD1, PD-L1 and PD-L2 in comparison to a control sample or reference value and administering a first therapy to the subject to treat the cancer and/or withholding the administration of the second therapy to the subject.

13. The method of embodiments 1-12, wherein the first therapy is a non-immunotherapeutic treatment or an immunotherapy, wherein the non-immunotherapeutic treatment or the immunotherapy is designed to kill and/or control the proliferation of cancer cells and the second therapy is designed to reduce the ability of the cancer cells to evade the immune system of the subject and is directed to activating the MHC class I transactivation pathway by activating the expression of nlrc5 or the expression and/or activity of NLRC5 mRNA or protein in the cancer cells.

14. The method of embodiment 13, wherein the immunotherapy comprises:
i) administering to the subject an agent that blocks a protein that inhibits the strength and duration of the immune response in the subject,
ii) adoptive cell transfer,
iii) administering to the subject a therapeutic antibody that causes the immune system-mediated destruction of the cancer cells,
iv) administering to the subject a non-antibody immune system molecule that causes the immune system-mediated destruction of the cancer cells,
v) administering to the subject a cancer vaccine, or
vi) administering to the subject an immune system modulator.

15. The method of embodiment 13, wherein the second therapy comprises administering to the subject:

a) an agent that causes the activation of NLRC5 protein activity;

b) a wild-type or mutant NLRC5 protein or a nucleotide encoding the wild-type or mutant NLRC5 protein;

c) an agent that causes a non-specific demethylation of genomic DNA; or

d) an agent that causes a site-specific demethylation of nlrc5 or a portion thereof.

16. The method of embodiment 13, wherein the first therapy is the non-immunotherapeutic treatment and the second therapy is not administered to the subject identified as having a cancer that is likely or not likely to evade the immune system of the subject.

17. The method of embodiment 13, wherein the first therapy is the non-immunotherapeutic treatment and the second therapy is not administered to the subject identified as having a cancer that is not likely to evade the immune system of the subject.

18. The method of embodiment 13, wherein the first therapy is the non-immunotherapeutic treatment and the second therapy is administered to the subject identified as having a cancer that is likely to evade the immune system of the subject.

19. The method of embodiment 13, wherein the first therapy is the immunotherapy and the second therapy is not administered to the subject identified as having a cancer that is not likely to evade the immune system of the subject.

20. The method of embodiment 13, wherein the first therapy is the immunotherapy and the second therapy is administered to the subject identified as having a cancer that is likely to evade the immune system of the subject.

21. The method of embodiment 13, wherein the first and second therapies are administered simultaneously or separately to the subject.

22. The method of embodiment 13, wherein the first therapy and/or the second therapy are administered in a sub-therapeutic amount.

23. The method of any preceding embodiment, wherein subjects having elevated/higher levels the aforementioned variables have an increase of at least 5%, 10%, 20%, 30%, 40%, 50%, 60%, 70%, 80%, 90% or 100% of the variables relative to expression levels in a control sample and subjects having reduced/lower levels the aforementioned variables have a decrease of at least 5%, 10%, 20%, 30%, 40%, 50%, 60%, 70%, 80%, 90%, or 100% of the variables relative to expression levels in a control sample.

24. The method of any preceding embodiment, wherein any combination of neoantigen load, mutation number, and expression of CTLA4, PD1, PD-L1, and PD-L2 are used in combination with the NLRC5 biomarker.

All patents, patent applications, provisional applications, and publications referred to or cited herein are incorporated by reference in their entirety, including all figures and tables, to the extent they are not inconsistent with the explicit teachings of this specification.

Following are examples which illustrate procedures for practicing the invention. These examples should not be construed as limiting. All percentages are by weight and all solvent mixture proportions are by volume unless otherwise noted.

### Example 1—Nlrc5 Expression in Cancer Tissues Induces CD8⁺ T Cell-Dependent Cytotoxicity

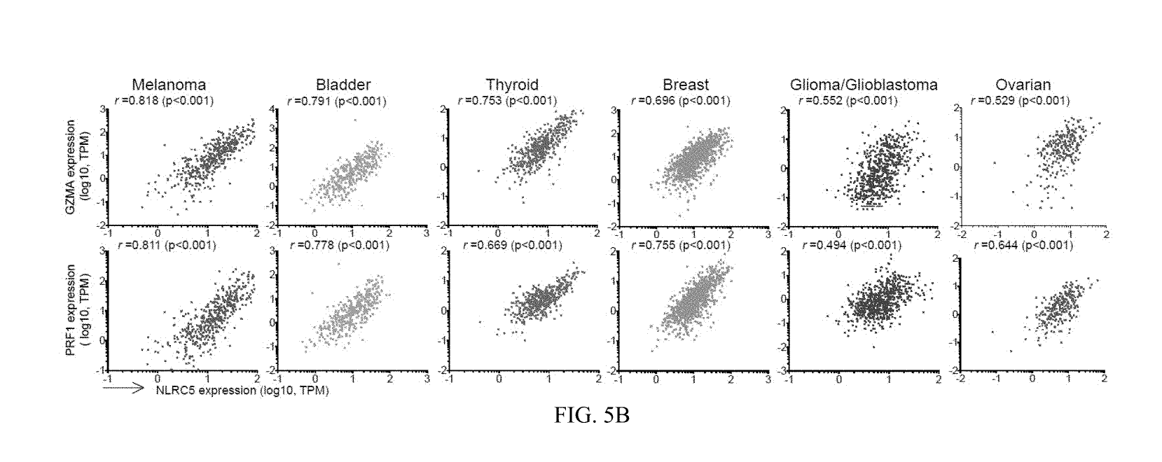

NLRC5 gene expression profile was examined in the biopsy samples from a cohort of 7747 solid cancer patients in The Cancer Genome Atlas (TCGA) database. The expression of hla-b highly correlated with nlrc5 expression level in the entire cohort (rₛ=0.753) (FIG. 1A). Correlation analysis for gene expression among 16 cancer types demonstrated that hla-b and nlrc5 expression showed high positive correlation (rₛ>0.70) in 11 cancer types and intermediate positive correlation (rₛ>0.50) in 4 cancer types (FIGS. 1B, C), with the highest correlation observed in melanoma. In addition to hla-b, expression of the hla-a, hla-c, b2m, lmp2, lmp7 (psmb8) and tap1 genes were also highly correlated with nlrc5 expression in melanoma and other cancers (FIG. 1D and FIG. 5A). Since NLRC5-mediated MHC class I expression is crucial for optimal activation and cytolytic activity of CD8⁺ T cells, the expression level of prf1 or gzma, which are known to be associated with cytotoxic T cell activity in cancer tissues, was also examined. The cohort of 20 solid cancer etiologies revealed a significant positive correlation between nlrc5 expression and PRF1 or GZMA (FIG. 1E and FIG. 5B). Although prf1 and gzma are expressed in both activated CD8⁺ T cells and NK cells, nlrc5 expression correlated only with cd8a, not the NK cell marker cd56 (FIG. 1F). These data indicate that nlrc5 expression in cancer tissues is critical for inducing CD8⁺ T cell-dependent cytotoxic activity, likely through the induction of MHC class I expression.

### Example 2—Methylation of Nlrc5, but not of Other MHC Class I Genes, is Selectively Used in Cancers to Evade Immunity

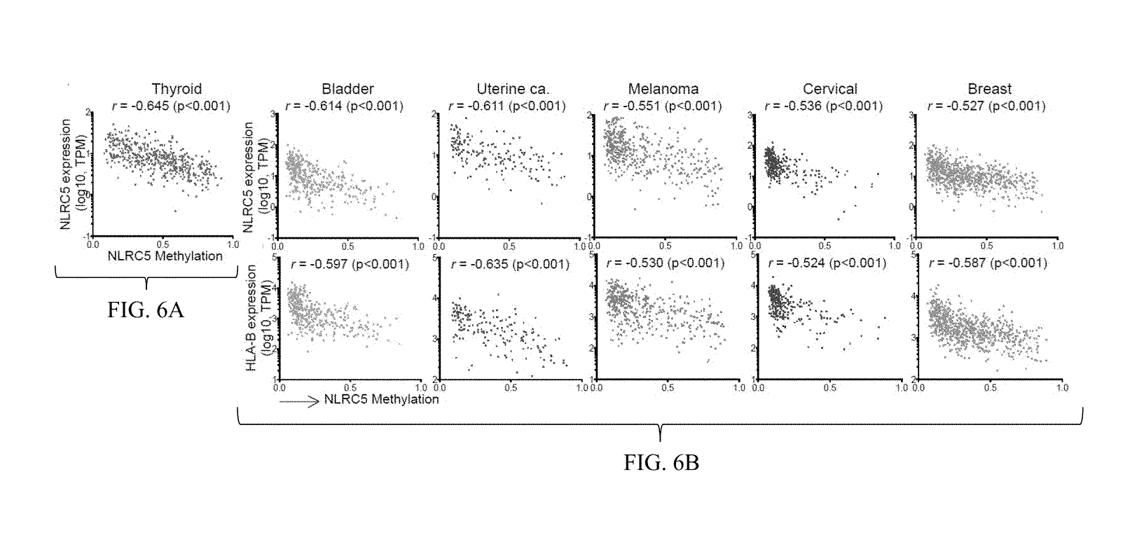

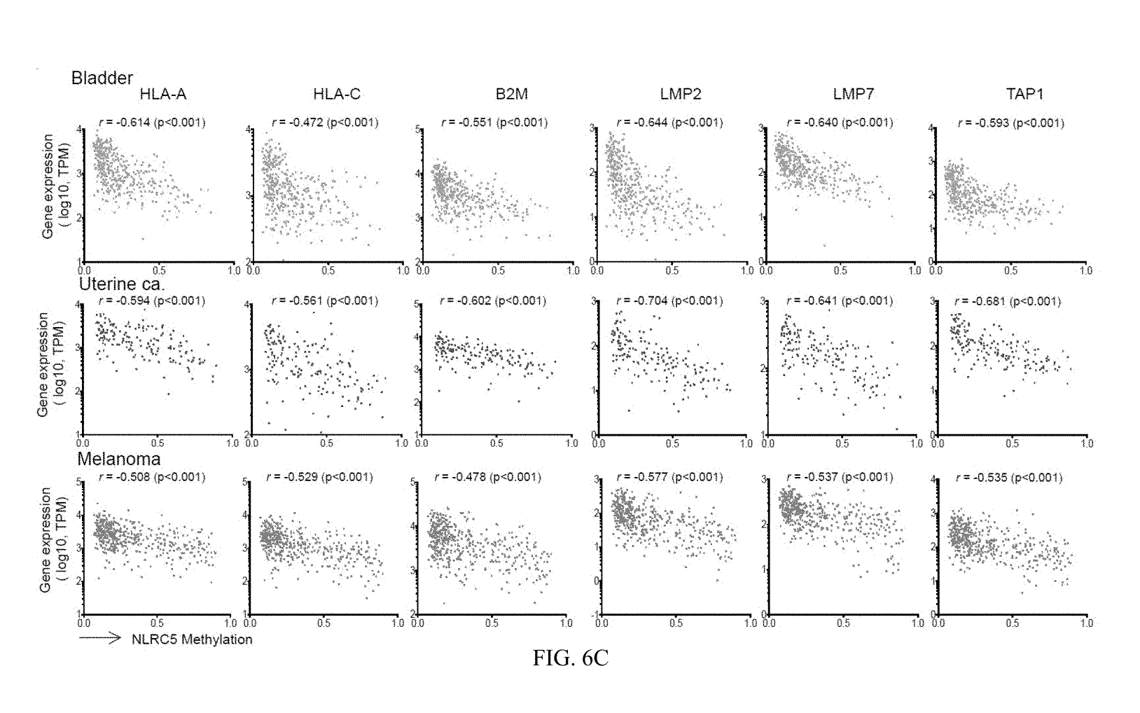

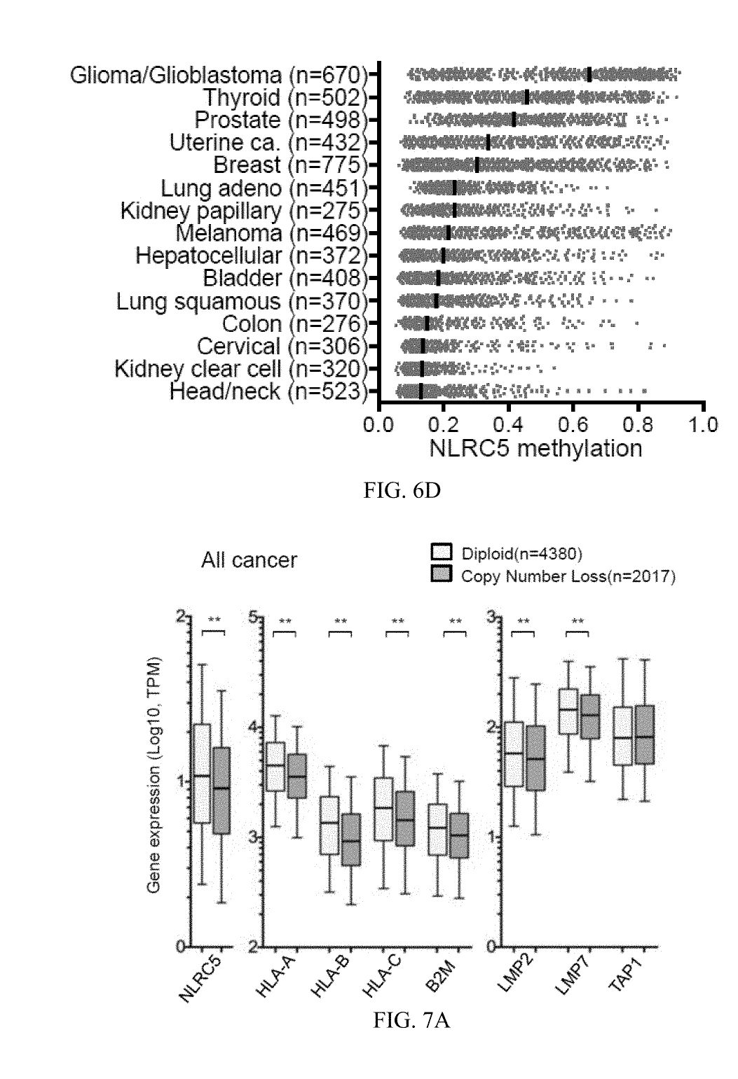

Epigenetic changes in cancer cells represent an important mechanism to alter gene expression in favor of cancer growth and immune evasion. Abnormal methylation of CpG islands in promoter regions can transcriptionally suppress genes which are unfavorable for cancer growth. Treatment of various cancer cell lines with a DNA-methylation inhibitor, 5-Azacitidine, resulted in the upregulation of nlrc5 and hla-b expression, suggesting that methylation of the nlrc5 promoter plays a role in the loss of MHC class I expression in cancer (FIG. 2A). Therefore, the level of DNA methylation at a CpG island in the nlrc5 promoter was quantified using a methylation-specific probe (FIG. 2B) and compared with the expression level of nlrc5. Analysis of biopsy samples from 6523 solid cancer patients revealed that methylation of the nlrc5 promoter negatively correlated with nlrc5 expression (rₛ=−0.585) (FIG. 2B). Suppression of nlrc5 expression by the promoter methylation was observed in 15 cancer types; an intermediate negative correlation (rₛ=−0.50 to −0.70) was found in 7 cancer types and a low negative correlation (rₛ=−0.30 to −0.50) in 8 cancer types (FIG. 2C and FIGS. 6A and 6B). Moreover, the methylation of the nlrc5 promoter negatively correlated with the expression of hla-b in all cancer types to various degrees (FIGS. 6A and 6B). nlrc5 promoter methylation also negatively correlated with the expression of hla-a, hla-c, b2m, lmp2, lmp7 and tap1 in thyroid and other cancers (FIG. 2D and FIG. 6C). Reduced expression of MHC class I genes was specifically correlated with nlrc5 methylation because methylation of the promoter for ciita did not correlate with the expression of hla-b (FIG. 2E). Strikingly, nlrc5 methylation negatively correlated with cd8aA, gzma and prf1, but not with cd56 (FIGS. 2F, G). Thus, methylation of nlrc5 in cancer cells results in the transcriptional suppression of nlrc5, leading to reduced expression of MHC class I genes and evasion of CD8⁺ cytotoxic T cell-dependent anti-tumor activity. Since hla methylation has also been reported in cancer cells, the methylation level of the nlrc5 promoter was compared with that of other MHC class I and related genes. While nlrc5 methylation was observed in different cancer types to various degrees (FIG. 6D), the DNA methylation was most severe in nlrc5 among class I related genes tested in thyroid cancer and the entire cancer cohort (FIG. 2H). Moreover, methylation of the nlrc5 promoter exhibited the most effective gene suppression among class I-related genes, because the negative correlation between DNA methylation and gene expression was most prominent for nlrc5, compared to all other MHC class I-related genes (FIGS. 2B, I and J). These data indicate that the methylation of nlrc5, but not of other MHC class I genes, is selectively used in various cancers as an immune evasion strategy for efficient suppression of the MHC class I pathway.

### Example 3—Cancer Cells Selectively Lose the Nlrc5 Gene at a High Frequency and Induce Reduced Expression of MHC Class I Genes

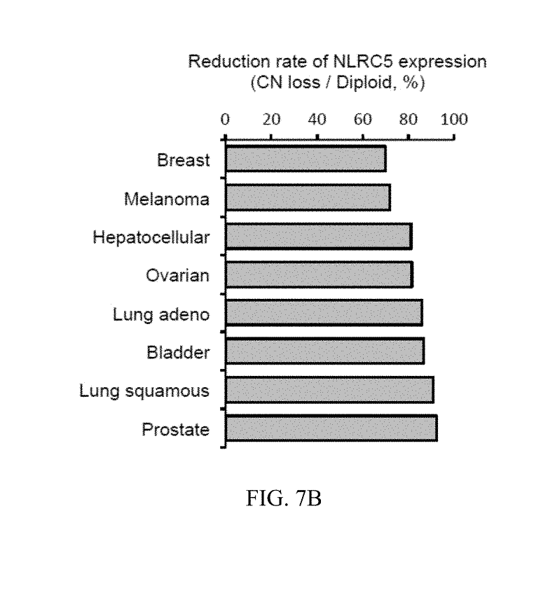

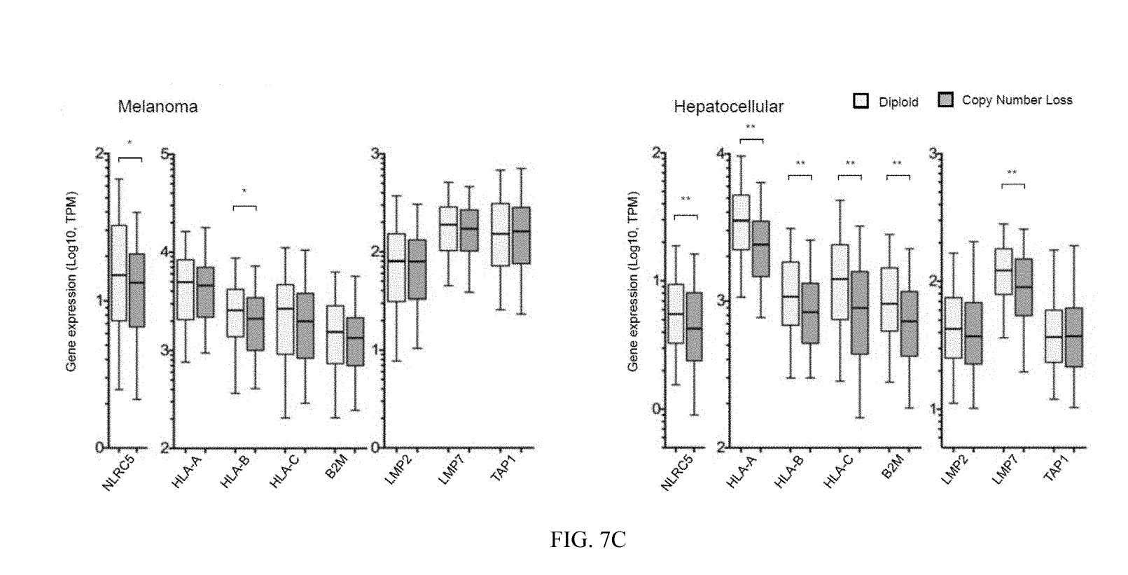

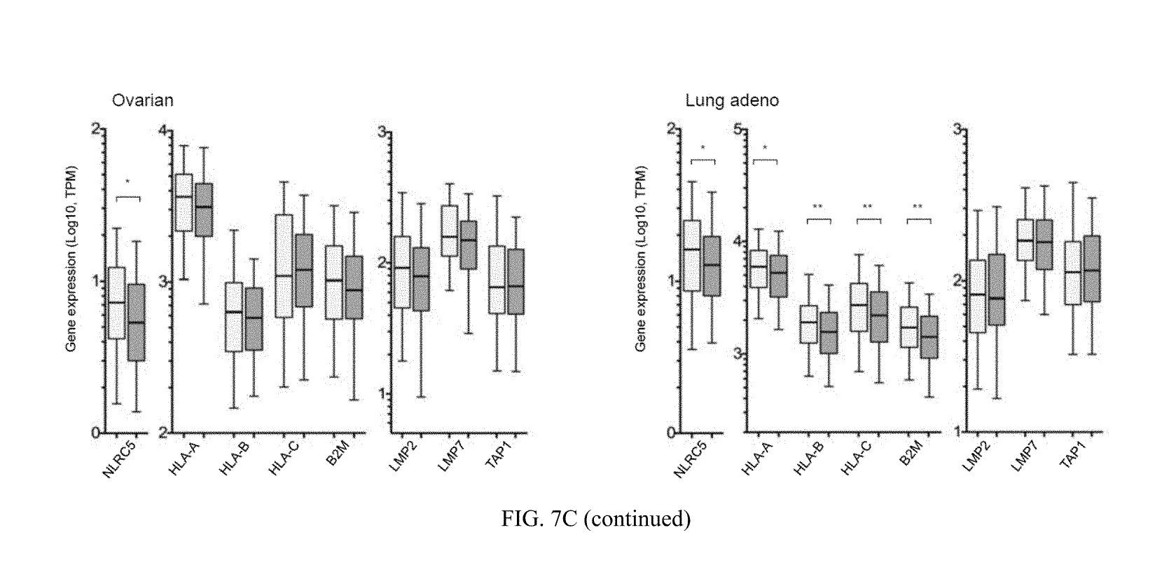

Changes of somatic gene copy number are frequently observed in cancer cells and associated with alteration of gene expression levels. The analysis of copy number in the cohort of 7730 cancer patients showed that all cancer types carry alterations in copy number of the nlrc5. Copy number loss (copy number=0 or 1) was observed in 28.6% of cancer patients, with the highest frequency in ovarian cancer patients (72.2%) (FIG. 3A). Remarkably, copy number loss occurred in nlrc5 at the highest frequency among MHC class I and related genes in the entire cancer cohort and in ovarian cancer, followed by b2m (FIG. 3B), again indicating that nlrc5 is a preferential target for cancer immune evasion among genes involved in the MHC class I pathway. Gene expression analysis demonstrated that patients with copy number loss showed reduced nlrc5 expression levels in the cancer cohort (FIG. 7A) as well as in cancers where the NLRC5 promoter is not methylated (FIG. 3C). In addition, patients with the nlrc5 copy number loss exhibited decreased expression of MHC class I and related genes, including hla-a, hla-b, hla-c, b2m, lmp2, and lmp7 (FIG. 3C and FIG. 7A). Various degrees of reduction of nlrc5 and class I gene expression were observed in samples of numerous cancers that had copy number loss, with the highest prevalence found in breast cancer (FIG. 3D and FIGS. 7B, C). These data indicate that cancer cells selectively lose the nlrc5 gene at a high frequency, resulting in reduced expression of MHC class I and related genes.

### Example 4—Cancer Cells Select the Inactivating Mutations in Nlrc5 and Reduce Expression of MHC Class I Genes

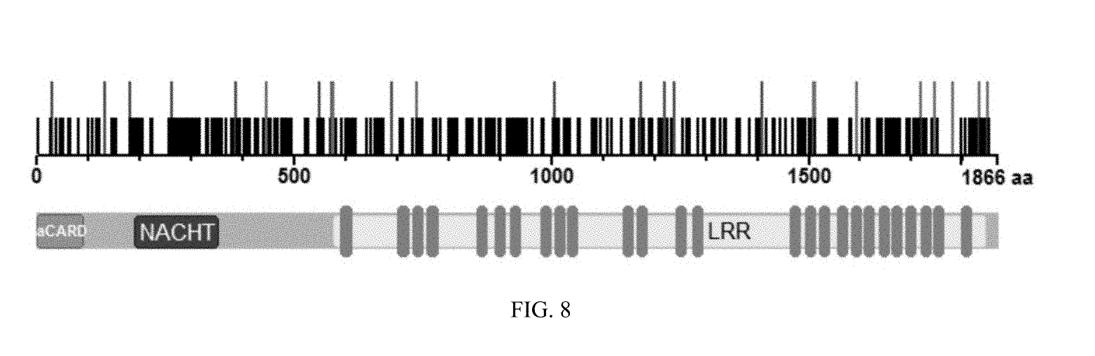

Since somatic mutations are important molecular mechanisms of carcinogenesis, biopsy samples from 7752 solid cancer patients were analyzed for somatic mutations in nlrc5. 142 patients were found to have mutations, most of which were missense mutations (58.5%) (FIG. 3E). Colon cancer patients exhibited the highest nlrc5 mutation rate (9.3%), followed by melanoma (7.0%) (FIG. 3F). Mutations were distributed across the entire nlrc5 coding region with no obvious hot spots (FIG. 8). To determine whether those mutations affect NLRC5 function, mutations (n=13) observed in more than one patient were analyzed for their ability to induce MHC class I gene expression via a reporter gene assay that employs the hla-b promoter and various nlrc5 expression vectors generated by site-directed mutagenesis (FIG. 3G). As shown in FIG. 3H, 7 out of the 13 nlrc5 mutants exhibited complete loss of induction for hla-b promoter activity, demonstrating that the majority of nlrc5 mutations in cancer patients are true loss-of-function mutations. Indeed, correlation analysis of hla-b and nlrc5 expression confirmed the tendency for reduced hla-b expression levels in patients with nlrc5 mutations compared to the wild-type (FIG. 3I). To further substantiate this observation with statistical analysis, the ratio of MHC class I genes to nlrc5 was plotted to reflect gene induction by NLRC5. As expected, the ratio of MHC class I to nlrc5 expression was decreased in the nlrc5 mutant group (FIG. 3J). These data indicate that nlrc5 mutations in cancer frequently result in the reduced expression of genes involved in MHC class I-mediated antigen presentation.

### Example 5—Nlrc5 Expression Correlates with Higher Survival in Cancer with the Exception of Brain Cancer

Since MHC class I expression and cytotoxic CD8⁺ T cell infiltration in tumors are involved in immunological defense in cancer patients, the effect of nlrc5 on overall survival was examined. Cancer patients were stratified into quartiles based on nlrc5 expression. The analysis of 5-year survival of patients with 20 cancer types revealed that the nlrc5 high expression quartile showed significantly better survival compared with the nlrc5 low expression quartile in 5 cancer types (melanoma, rectal cancer, bladder cancer, cervical cancer and head/neck cancer) (FIG. 4A). Among these, melanoma and bladder cancer displayed the most striking differences, with 5-year survival rates of 36% and 34% in the nlrc5-low group compared with 71% and 62% in the nlrc5-high group, respectively. Kaplan-Meier survival analysis also demonstrated that high nlrc5 expression was associated with significantly improved cumulative survival in melanoma, bladder cancer and cervical cancer (FIG. 4B). In addition to NLRC5, the expression of NLRC5-dependent (hla-a, hla-c, b2m, lmp2, lmp7 and tap1, FIG. 4C) but not NLRC5-independent (calreticulin, tapasin, erp57, erap1, FIG. 4D) genes involved in MHC class I antigen presentation was positively associated with cumulative survival of melanoma patients. The expression of markers for cytotoxic CD8⁺ T cell activity (cd8a, gzma, and prf1, FIG. 4E) but not NK cell activity (cd56, FIG. 9A) also correlated with better cancer patient survival, most likely through NLRC5-dependent MHC class I antigen presentation. High methylation of nlrc5 but not of other MHC class I and related genes (hla-a, hla-c, b2m, lmp2, lmp7 and tap1) was associated with poor survival in melanoma and bladder cancer, indicating that aberrant epigenetic changes specifically in the nlrc5 in cancer cells impacted clinical outcomes (FIG. 4F and FIGS. 9B, C). Intriguingly, brain cancer (glioma/glioblastoma) showed an opposite correlation with the high nlrc5 expression cohort exhibiting a significantly lower 5-year survival rate. Although the exact mechanism is uncertain, this effect might be due to the unique anatomy of the brain. Because brain mass is limited by the skull, unlike other cancers, one major life-threatening complication of brain tumors is the development of brain edema, which is associated with inflammatory events including impaired blood-brain barrier and destruction of normal brain tissues. In fact, patients with brain tumors are commonly treated with anti-inflammatory drugs such as corticosteroids. Thus, nlrc5 expression is correlated with higher survival in multiple cancer types, with the exception of brain cancer in which it appears to be a negative prognostic factor.

### Example 6—NLRC5 Expression in Patients that Respond to Checkpoint Blockade Immunotherapy

### Methods

### Data Sets

The cohort for the analysis of response to anti-CTLA4 therapy (ipilimumab) was obtained through Database of Genotypes and Phenotypes (dbGaP) (Mailman et al., 2007; Tryka et al., 2014), accession number phs000452.v2.p1 (Van Allen et al., 2015).

Data for survival analysis of melanoma was obtained through the Cancer Genome Atlas (TCGA) data portal (Worldwide Website: tcga-data.nci.nih.gov/tcga), Skin Cutaneous Melanoma (SKCM). Gene expression data (mRNASeq; illuminahiseq_rnaseqv2-RSEM genes), DNA methylation data (humanmethylation450-within_bioassay_data_set_function) and clinical data (Merge_Clinical) were accessed through GDAC Firehose (gdac.broadinstitute.org). Somatic mutation data were accessed through the cBioPortal (Cerami et al., 2012).

Patient Profiling of dbGap Dataset

A patient population of 42 total melanoma patients with metastatic melanoma who had taken ipillimumab monotherapy was analyzed. Patients were stratified by clinical benefit status as described previously (Van Allen et al., 2015). Response to ipilimumab was defined as CR (complete response) or PR (partial response) by Response Evaluation Criteria in Solid Tumors (RECIST) criteria or SD (stable disease) by RECIST criteria (Eisenhauer et al., 2009) with overall survival greater than 1 year. Non-response to ipilimumab was defined as PD (progressive disease) or SD (stable disease) by RECIST criteria with overall survival less than 1 year.

### Gene Expression Analysis

After getting access to dbGap, the datasets were downloaded and converted to FastQ file format using SRA Toolkit v2.6.3. Paired-end reads for 42 samples were checked to trim for low quality bases using Trimmomatic (Bolger et al., 2014). Filtered reads were mapped to the GRCh37/hg19 assembly using TopHat v2.0.13 (40). HTSeq (Anders et al., 2015) was then used to generate raw read counts per gene using intersection-nonempty parameter to account for ambiguous read mappings. Gene expression values were generated for further analyses using DESeq2 (Love et al., 2014), following recommended guidelines by the authors. The expression levels of NLRC5, HLA-B, B2M, CD8A, granzyme A (GZMA), perforin (PRF1) and CD56 measured by RNA sequencing (RNA-seq) were compared between responders and non-responders to ipilimumab using the Mann-Whitney U Test.

### Gene Set Enrichment Analysis (GSEA)

Gene Set Enrichment Analysis (GSEA, website: broad.mit.edu/gsea/) was used to assess differential expression of NLRC5 related MHC Class I genes between response and non-response groups (Subramanian et al., 2005). The expression values for all genes in the cohort were placed into the required format. The cohort was separated into two consisting of response and nonresponse groups to the anti-CTLA4 therapy. The log 2 transform of normalized counts from RNA sequence data was used for the expression values of genes. The gene set tested used was based on knowledge of the literature concerning NLRC5 and the MHC class I antigen presentation pathway (Kobayashi et al., 2012; Yoshihama, 2016; Yoshihama et al., 2017). Pseudo genes were excluded as well as others determined to not be related closely enough to NLRC5 or MHC Class I.

### Mutation and Neoantigen Analysis

Numbers of mutation and neoantigen (mutation load and neoantigen load, respectively) were counted from the data of 35 melanoma patients treated with ipilimumab, generated by Van Allen et al. (2015). Those were compared between responders and non-responders to ipilimumab using the Mann-Whitney U Test. The bidimensional combination of NLRC5 expression and mutation or neoantigen load was assessed and p-values were calculated using Hotelling's T² Test to compare between responder and non-responder to ipilimumab. Next, to evaluate the influence of those variables for response to ipilimumab, cohort was divided into four groups based on the level of NLRC5 expression and mutation or neoantigen load and was calculated for the response rate to ipilimumab. Patients carrying higher value of the median are defined as high group, those carrying lower value of the median are defined as low group in respective variables. Statistical significance between the groups of high NLRC5 expression/high mutation or neoantigen load and low NLRC5 expression/low mutation or neoantigen load was determined by the χ2 test. Expression values for other genes known to predictors of response to anti-CTLA4 therapies, such as CTLA-4, PD-1, PD-L1 and PD-L2 were combined with NLRC5 expression and mutation or neoantigen load and represented in a three-dimensional scatterplot.

### Logistic Regression Analysis

Logistic regression models were fitted with different combinations of the following covariates: the values for expression of 5 genes (NLRC5, PDL1, PDL2, CTLA4 and PD1), mutation load, and neoantigen load. Up to 3 of these variables were considered for the regression models at a time. Samples with missing values were eliminated before fitting the regression. Multicollinearity was assessed through calculation of variance inflation factors and Pearson's correlation coefficient. A scatterplot matrix was created with fitted curves and regression lines and the distribution of each variable was inspected. An ROC curve was generated for each combination of covariates using the pROC package (version 1.8) in R. The training data was used as the prediction data for the ROC curves. Threshold values were determined at points where the sensitivity is 100%. These curves were plotted and a selection were reported. A bootstrapping procedure with 10,000 repetitions was used to estimate 95% confidence intervals for the curves as well as calculate a mean AUC. This was accomplished by sampling the cohort with replacement to create new groupings of data (the same size as the original cohort) then used to construct ROC curves. The AUC was calculated for each of these new curves. The confidence interval was determined by ordering the AUCs by value and returning the value at the index 2.5% of the length of the list away from the beginning and end. The standard error (SE) was calculated (Hanley et al., 1982). The best prediction model was chosen based on the highest mean AUC.

### Survival Analysis

Cox proportional hazards model was used to analyze the survival of a cohort of melanoma patients (n=458) obtained from TCGA. The model included age, cancer stage, mutation load, NLRC5 expression and NLRC5 methylation as covariates. Survival curves were created depicting the difference in survival between the groups through division of the cohort into top and bottom 50% based on NLRC5 expression, NLRC5 methylation, and mutation load. Patients were stratified in a similar fashion by two variables (NLRC5 expression and mutation load) yielding four groups (high NLRC5 expression/high mutation load, high/low, low/high, low/low, respectively). The same was also performed for NLRC5 methylation and mutation load (high NLRC5 methylation/high mutation load, high/low, low/high, low/low, respectively). Interaction terms for continuous variables were assessed for each possible combination.

### Statistical Analysis

Statistical analysis was performed using R and Graph Pad Prism software.

### Results

### High NLRC5 Expression in Melanoma Patients Who Responded to Anti-CTLA4 Blockade Immunotherapy.

Immunotherapy using checkpoint inhibitors such as anti-CTLA4, anti-PD-1/PD-L1 antibodies are emerging and promising cancer treatments (Pardoll, 2012; Sharma et al., 2015). Since these therapies rely on the induction of effective anti-tumor immune responses mediated by T cells, the efficacy would be limited if cancer cells are able to evade the immune system.

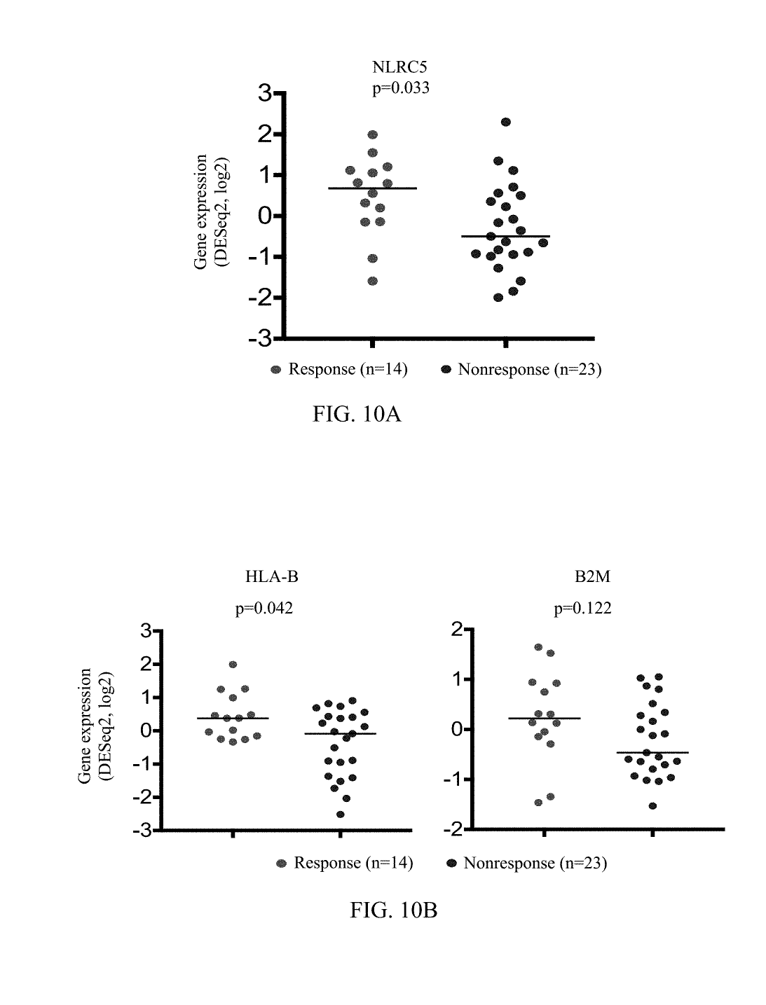

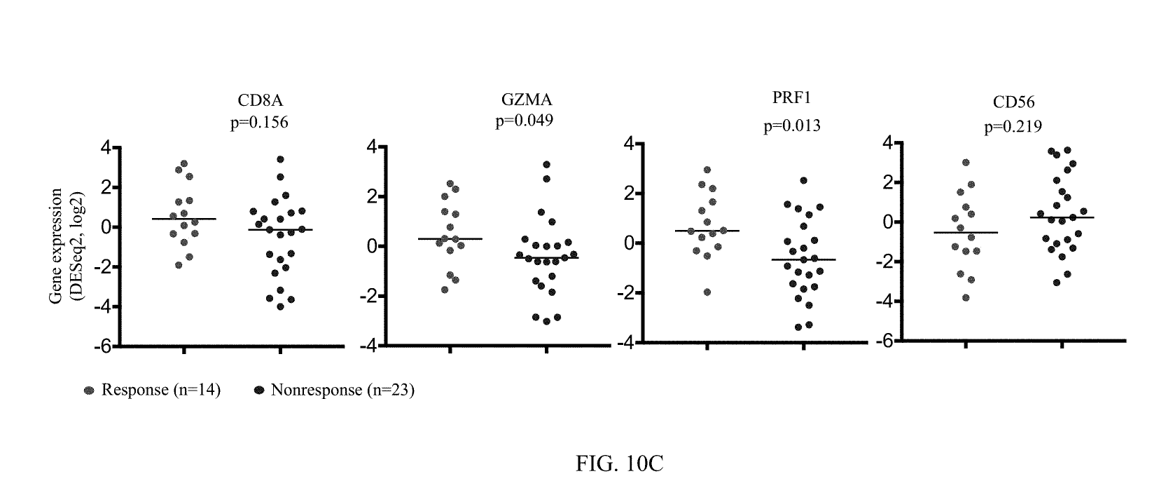

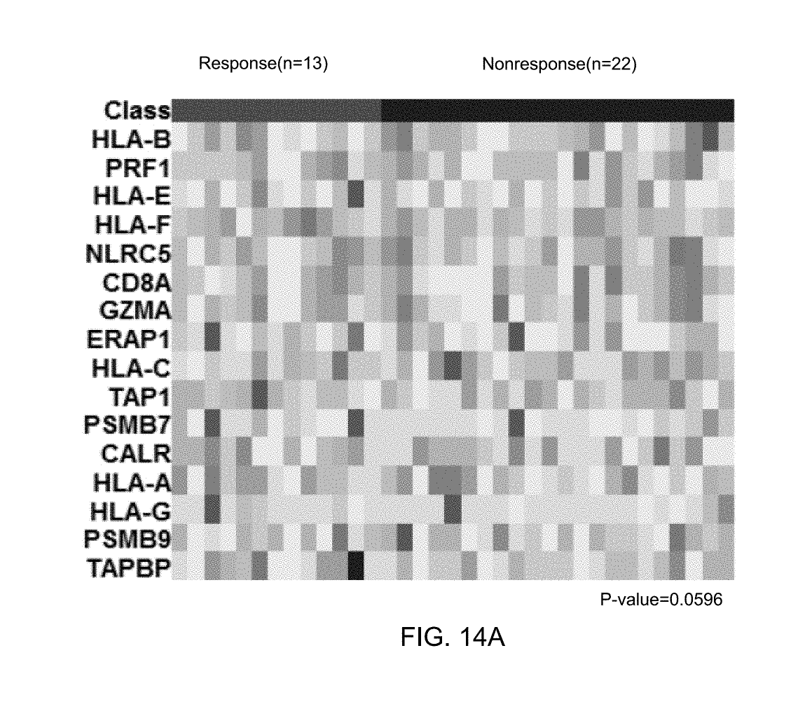

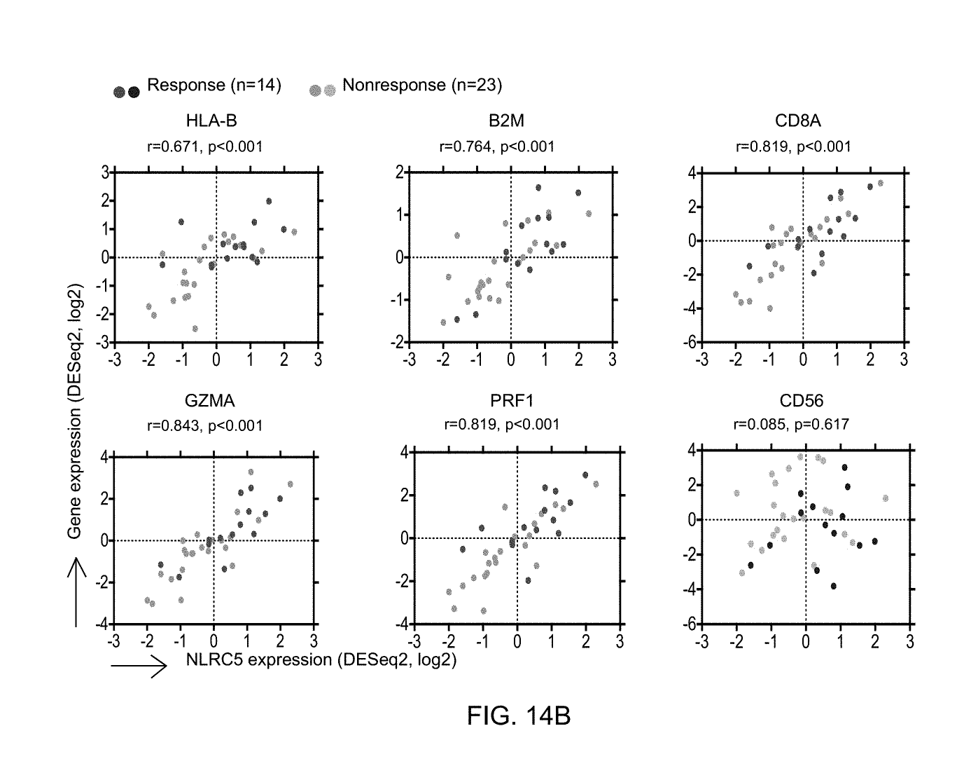

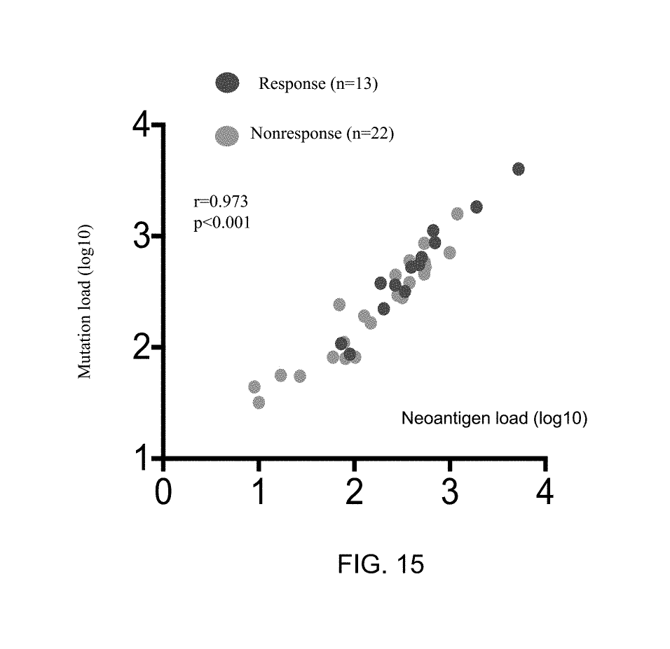

Among the melanoma patient cohort who received anti-CTLA4 checkpoint blockade therapy, we have analyzed and compared gene expression level between the groups who benefited from the treatment (responder) and who did not (non-responder). Gene set enrichment analysis indicated that a set of various MHC class I related genes was differentially expressed between responders and non-responders (FIG. 14). Among these, we found that NLRC5 expression is significantly elevated in the group who showed a benefit from the anti-CTLA4 therapy (FIG. 10A). The expression of NLRC5 was correlated with HLA-B and B2M in this melanoma patient cohort (FIG. 14B). Indeed in addition to NLRC5, a responder group exhibited higher expression of HLA-B than a non-responder group and B2M showed a similar tendency although it was not statistically significant with this cohort size. The expression of NLRC5 was also correlated with the expression level of CD8A and Granzyme A (GZMA)/Perforin (PRF1), markers for CD8 T cell activation but not CD56, a marker for NK cells in various cancers and in this melanoma cohort (FIG. 14B). The responder group exhibited higher expression of GZMA and PRF1 (FIG. 10C). Although GZMA and PRF1 are expressed in both CD8+ T cells and NK cells, the high expression of GZMA and PRF1 was likely due to activated CD8 T cells rather than NK cells since the responder group did not exhibit higher expression of CD56. These data suggest that NLRC5 and NLRC5-mediated MHC class I dependent CD8 T cell activation is important for the effective responses to anti-CTLA4 checkpoint blockade immunotherapy.

### NLRC5 Expression and Neoantigen Loads are Independent Predictors of Clinical Responses to Anti-CTLA4 Therapy.

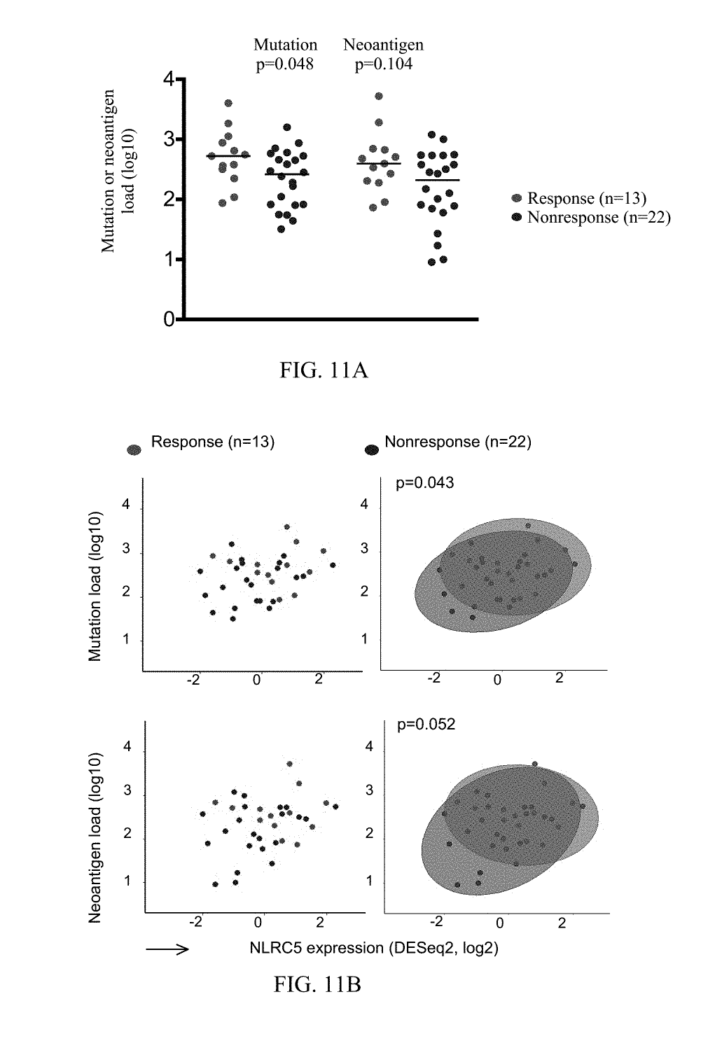

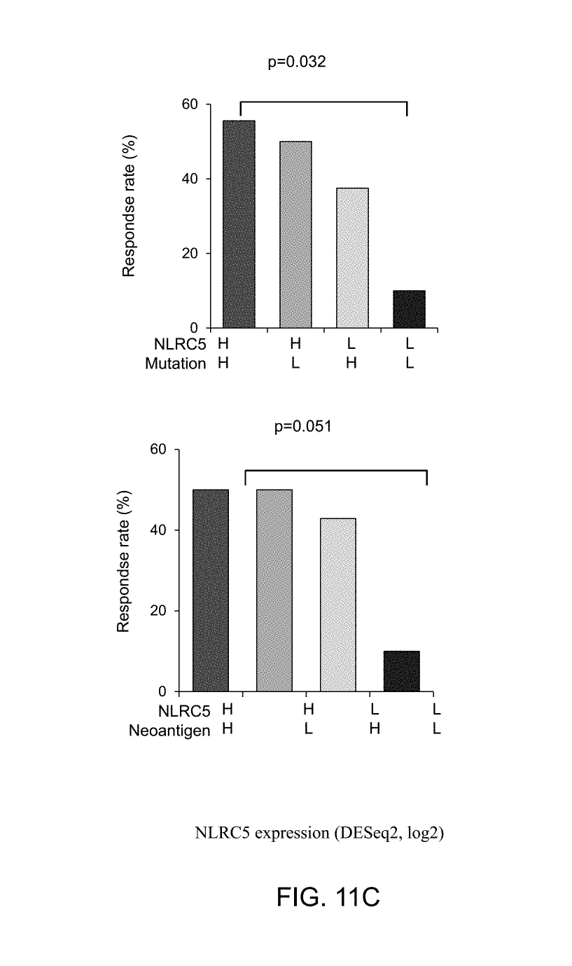

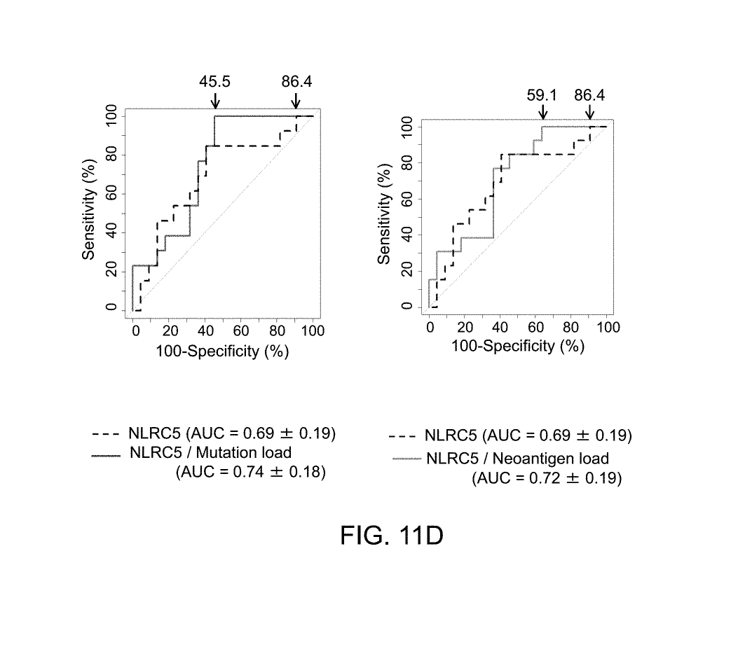

In order to test if the addition of mutation/neoantigen load to NLRC5 expression would improve predictions, we performed multivariate analysis by logistic regression using these variables. The cohort of this study showed higher neoantigen loads or mutation number in the responder group (FIG. 11A). Scatter plots for NLRC5 expression combined with neoantigen loads, or mutation number showed that non-responder groups, in particular the patient group who showed low NLRC5 expression and low neoantigen loads or mutation numbers, were more clearly separated from responder groups (FIG. 11B). Patients were then stratified by NLRC5 expression and neoantigen load or mutation numbers, yielding four groups (high/high, high/low, low/high, and low/low). The response rate in the group with low NLRC5 expression and low neoantigen load (or low mutation number) was significantly less than that of the group with high NLRC5 expression and high neoantigen load (or high mutation number) (FIG. 11C). These results suggest that two variables, NLRC5 expression and neoantigen loads (or mutation number) may be useful to identify non-responders. We further performed ROC curve analysis for logistic regression model using those variables. False positive rate with 100% sensitivity by single variable (NLRC5 expression alone) was 86.4% and was improved to 45.5% by two variables (NLRC5 expression and mutation numbers) or 59.1% (NLRC5 expression and neoantigen load) (FIG. 11D). These data indicate that analysis with two variables are useful to identify the patient population who do not respond to anti-CTLA4 therapy.

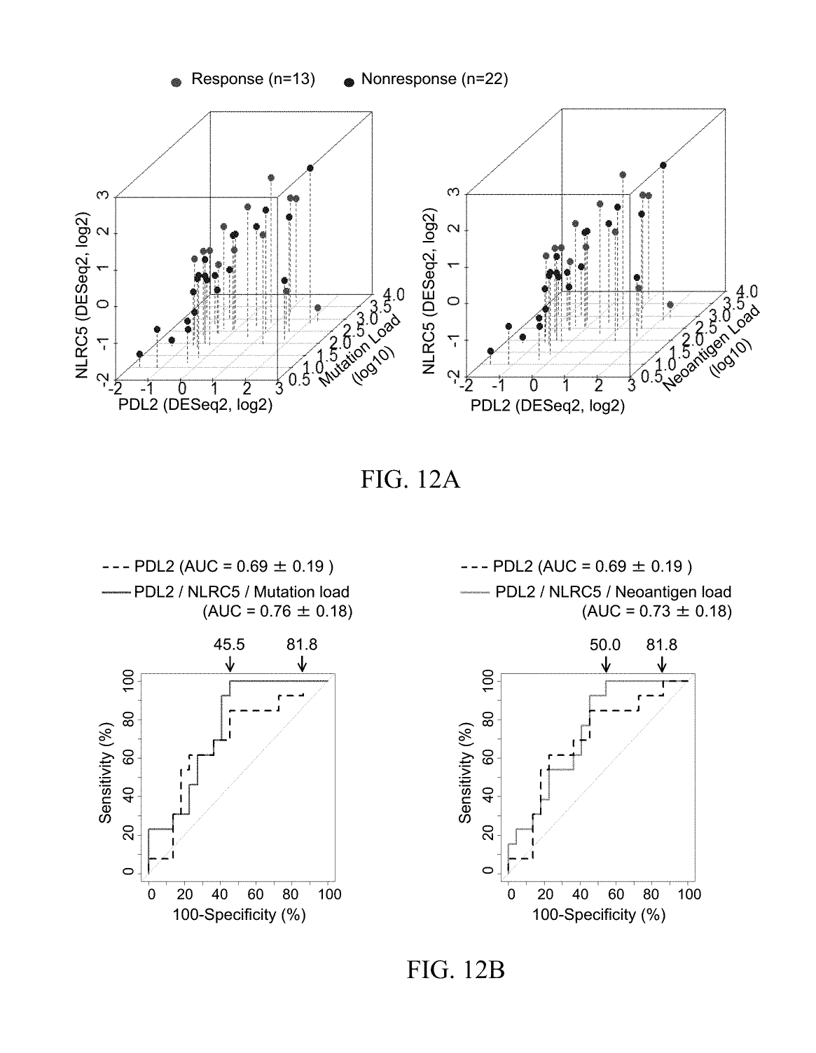

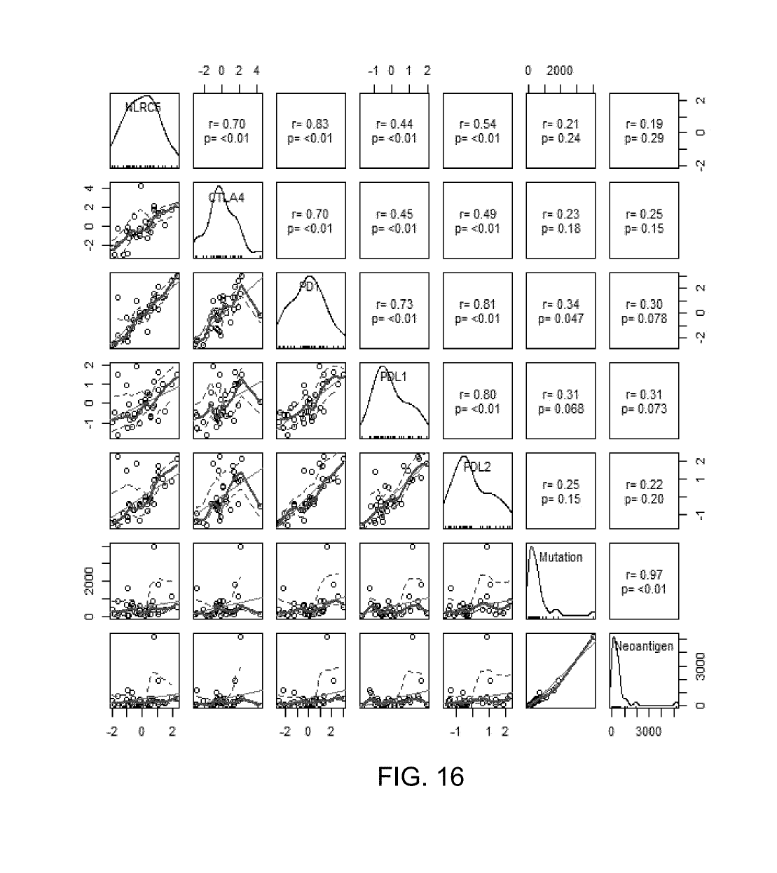

The expression of NLRC5 exhibited intermediate to high correlation with the expression of CTLA4 (Pearson's correlation coefficient 0.70) and PD1 (0.83), while NLRC5 expression with expression of PDL1 (0.44) and PDL2 (0.54) was low (FIG. 16), suggesting that CTLA4 and PD1 might not be good variables partnered with NLRC5. Indeed, upon ROC curve analysis for logistic regression model using single (NLRC5 expression), double (NLRC5 expression plus mutation load) or triple (NLRC5 expression plus mutation load plus either CTLA4, PD1, PDL1 or PDL2 expression), AUC with triple variable using NLRC5 expression, mutation load and PDL2 expression was highest among all possible combinations (Table 4). Scattered plots with NLRC5 expression, PDL2 expression and mutation load/neoantigen load showed a part of the non-responder group did not overlap with the responder group (FIG. 12A). ROC curve analysis using these variables showed that improvement of false positive rate with 100% sensitivity from 81.8% by a single variable (PDL2 expression alone) to 45.5% by three variables (PDL2, NLRC5 expression and mutation load) or 50.0% (PDL2, NLRC5 expression and neoantigen load) (FIG. 12B). These indicate that the analysis with three variables are useful to identify the patient population who do not respond to anti-CTLA4 therapy.

### Survival Analysis Using NLRC5 Expression and Mutation Number

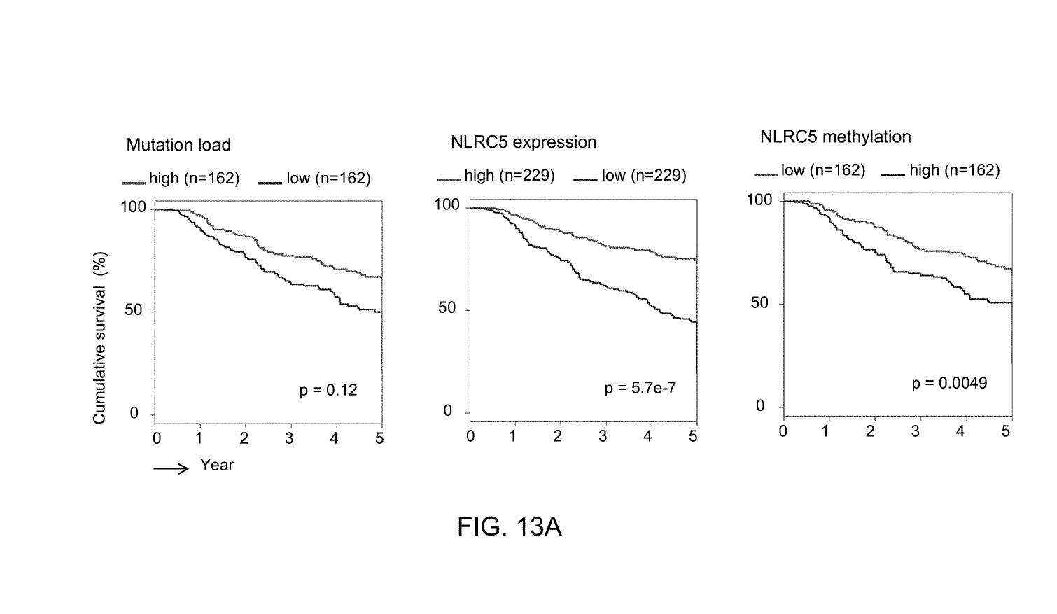

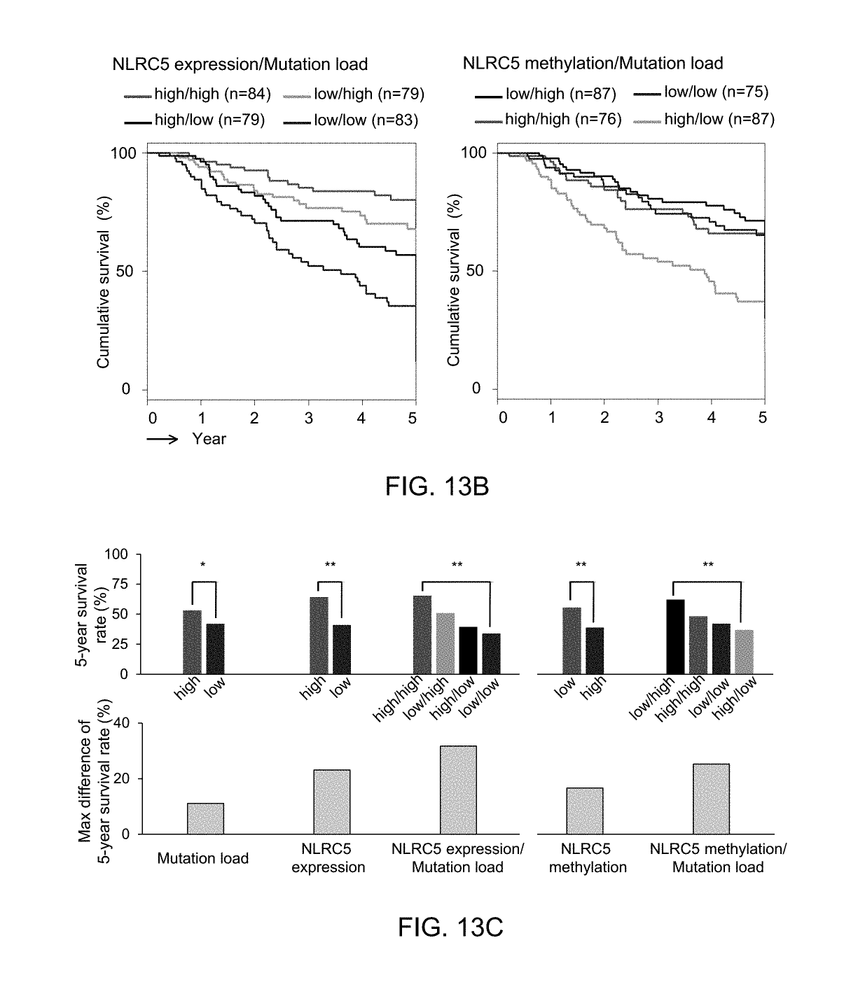

The multivariable logistic regression including NLRC5 expression together with mutation load or neoantigen load indicated that the analysis of two variables would be superior to predict response to anti-CTLA4 checkpoint blockade therapy (FIGS. 11B-D). Since these variables are critical for immune surveillance against cancer, we hypothesized that an association would be observed with patient prognosis and overall survival. Using melanoma patient data from the TCGA database, we performed survival curve analysis using a multivariate Cox proportional hazards model. The cohort was divided into two groups with values higher or lower than the median for the variables of mutation load, NLRC5 expression and NLRC5 promoter methylation (n=328, 458 and 328, respectively). The high mutation patient group demonstrated trends of better prognosis than the low mutation group, although this trend was not statistically significant (p=0.12) (FIG. 13A). The groups of high NLRC5 expression and low NLRC5 methylation showed significantly better prognosis than low NLRC5 expression group and high NLRC5 methylation group respectively (p=5.7e-7 and p=0.0049) (FIG. 13A). Survival curve analysis of four groups divided by the level of NLRC5 expression and mutation load demonstrated different survival curves based on NLRC5 expression level and mutation load, with the high NLRC5 expression/high mutation load group showing better prognosis than the low NLRC5 expression/low mutation load group (FIGS. 13B-C). Similarly, survival curve analysis for four groups divided by the level of NLRC5 promoter methylation and mutation load showed that NLRC5 methylation high/mutation low group is a high risk group with poor prognosis and NLRC5 methylation low/mutation high group is a low risk group with better prognosis (FIGS. 13B-C). Interaction terms were non-significant except for mutation load paired with NLRC5 methylation. Taken together, these data indicate that multivariate analysis using NLRC5 expression/methylation status with mutation load is superior to single variable analysis and may be of value as prognostic biomarkers in melanoma.

### Discussion

Discovery of inhibitory receptors on T cells and development of monoclonal antibodies against them had led to wide usage of checkpoint blockade therapy in various cancers. Although these therapies are effective to many cancer patients, complete response rate is ranging around ˜20% for anti-CTLA4 antibody therapy (Schadendorf et al., 2015; Maio et al., 2015) and ˜30% for anti-PD/anti-PD-L1 therapy in the case of melanoma. These treatments are quite expensive and if ineffective, this will leave significant financial burden to the patients and heath care system.

This study showed that NLCR5 is a novel biomarker to predict outcome of CTLA4 blockade therapy. Although NLRC5 expression alone has weak prediction power (FIG. 10), its combination with other variables yielded improved performance (FIG. 11). In particular, NLRC5 expression and neoantigen load/mutation number exhibit a low degree of multicollinearity and are weakly correlated (Pearson's coefficient 0.19 and 0.21, respectively, FIG. 16). Combining NLRC5 expression and mutation numbers demonstrated better AUC and a lower false positive rate at 100% (FIG. 11D). These data suggest that the combination of NLRC5/mutation load is superior to these variables alone in identifying non-responders. In contrast to the low correlation between NLRC5 expression and mutation number, the expression of CTLA4, PD1, PD-L1 or PD-L2 relative to NLRC5 expression carry high to intermediate correlation (Pearson's coefficient 0.70, 0.83, 0.44 or 0.54). It appeared that PD-L2 is the best consideration to combine with NLRC5 and mutation number (FIG. 12 Although this study concerned only melanoma patients who received anti-CTLA4 checkpoint therapy, anti-PD-1/PD-L1 antibody therapy use similar mechanisms to increase anti-tumor activity. Thus it is feasible that NLRC5 expression/mutation number biomarker might also be useful for cancer patients treated with anti-PD-1/PD-L1 antibody therapy. Checkpoint blockade therapy was initially tried in melanoma patients, but they have been expanded to a dozen cancer types including lung, breast and kidney. Therefore, investigations into the role of NLRC5 expression and the possibility of mutation load/NLRC5 expression for prediction with regards to these cancers would be of interest.

In summary, this study identified the expression of NLRC5 as a novel predictive biomarker and multivariate analysis using NLRC5 seemed of significant value to predict patient response to checkpoint blockade therapy.

TABLE 4
AUC values and corresponding false positive
rate in different combinations of variables

| | | False Positive | Mean | Boot- |
|---|---|---|---|---|
| | | Rate when | Boot- | strapped |
| | | True Positive | strapped | 95% Conf. |
| Variables | AUC | Rate is 100% | AUC | Int. |
| NLRC5* | 0.69 ± 0.19 | 0.8636 | 0.69 | (0.43, 0.90) |
| NLRC5*/ | 0.74 ± 0.18 | 0.5909 | 0.76 | (0.49, 0.86) |
| Mutation load | | | | |
| NLRC5*/ | 0.72 ± 0.19 | 0.5909 | 0.74 | (0.43, 0.90) |
| Neoantigen load | | | | |
| NLRC5*/ | 0.72 ± 0.18 | 0.5000 | 0.78 | (0.58, 0.93) |
| Mutation load/ | | | | |
| CTLA4* | | | | |
| NLRC5*/ | 0.75 ± 0.18 | 0.6818 | 0.79 | (0.60, 0.93) |
| Mutation load/ | | | | |
| PD1* | | | | |
| NLRC5*/ | 0.75 ± 0.18 | 0.4545 | 0.78 | (0.59, 0.93) |
| Mutation load/ | | | | |
| PDL1* | | | | |
| NLRC5*/ | 0.76 ± 0.18 | 0.4545 | 0.80 | (0.60, 0.95) |
| Mutation load/ | | | | |
| PDL2* | | | | |

*Gene expression (DESeq2, log2)

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated within the scope of the invention without limitation thereto.

### REFERENCES

Anders S, Pyl P T, & Huber W (2015) HTSeq—a Python framework to work with high-throughput sequencing data. Bioinformatics 31(2): 166-169.
Bolger A M, Lohse M, & Usadel B (2014) Trimmomatic: a flexible trimmer for Illumina sequence data. Bioinformatics 30(15):2114-2120.
Cerami, E. et al. The cBio cancer genomics portal: an open platform for exploring multidimensional cancer genomics data. Cancer discovery 2, 401-404, doi:10.1158/2159-8290.CD-12-0095 (2012).
Deshpande et al., Current trends in the use of liposomes for tumor targeting, Nanomedicine (Lond); 8(9):doi:10.2217/nnm.13.118 (2013).
Eads, C., Laird P. Combined bisulfite restriction analysis (COBRA). Methods Mol Biol. 200:71-85 (2002).
Ehrich, M., Nelson, M., Stanssens, P., Zabeau, M., Liloglou, T., Xinarianos, G., Quantitative high-throughput analysis of DNA methylation patterns by base-specific cleavage and mass spectrometry. Proc Natl Acad Sci USA, 2005; 102:15785-90.
Eisenhauer E A, et al. (2009) New response evaluation criteria in solid tumours: revised RECIST guideline (version 1.1). European journal of cancer 45(2):228-247.
Hanley J A & McNeil B J (1982) The meaning and use of the area under a receiver operating characteristic (ROC) curve. Radiology 143(1):29-36.
Hilton et al. (2015), epigenome editing by a CRISPR/Cas9-based acetyltransferase activates genes from promoters and enhancers, Nat Biotechnol; 33(5): 510-517.
Hochhauser et al. (2007), Modulation of topoisomerase IIA expression by a DNA sequence-specific polyamide, Mol Cancer Ther; 6(1):346-354.
Ji et al. (2014), Engineered zinc-finger transcription factors activate OCT4 (POU5F), SOX2, KLF4, c-MYC (MYC) and miR302/367, Nucleic Acids Research, Vol. 42, No. 10, pp. 6158-6167.
Kobayashi, K. S. & van den Elsen, P. J. NLRC5: a key regulator of MHC class I-dependent immune responses. Nat Rev Immunol 12, 813-820 (2012).
Love M I, Huber W, & Anders S (2014) Moderated estimation of fold change and dispersion for RNA-seq data with DESeq2. Genome biology 15(12):550.
Maeder et al. (2013), Targeted DNA demethylation and endogenous gene activation using programmable TALE-TET1 fusions, Nat Biotechnol. 2013 December; 31(12): 1137-1142.
Maherani et al., Liposomes: a review of manufacturing techniques and targeting strategies, Current Nanoscience; 7:436-452 (2011).
Mailman M D, et al. (2007) The NCBI dbGaP database of genotypes and phenotypes. Nature genetics 39(10):1181-1186.
Maio M, et al. (2015) Five-year survival rates for treatment-naive patients with advanced melanoma who received ipilimumab plus dacarbazine in a phase III trial. Journal of clinical oncology: official journal of the American Society of Clinical Oncology 33(10):1191-1196.
Meissner, T. B. et al. NLR family member NLRC5 is a transcriptional regulator of MHC class I genes. Proceedings of the National Academy of Sciences of the United States of America 107, 13794-13799 (2010).
Paul, C., Clark, S. Cytosine methylation: quantitation by automated genomic sequencing and GENESCAN analysis. Biotechniques. 1996; 21:126-33.
Pardoll D M (2012) The blockade of immune checkpoints in cancer immunotherapy. Nature reviews. Cancer 12(4):252-264.
Schadendorf D, et al. (2015) Pooled Analysis of Long-Term Survival Data From Phase II and Phase III Trials of Ipilimumab in Unresectable or Metastatic Melanoma. Journal of clinical oncology: official journal of the American Society of Clinical Oncology 33(17):1889-1894.
Sharma, P. & Allison, J. P. The future of immune checkpoint therapy. Science 348, 56-61, doi:10.1126/science.aaa8172 (2015).
Subramanian A, et al. (2005) Gene set enrichment analysis: a knowledge-based approach for interpreting genome-wide expression profiles. Proceedings of the National Academy of Sciences of the United States of America 102(43): 15545-15550.
Thakore et al. (2016), Editing the epigenome: technologies for programmable transcription and epigenetic modulation, Nature Methods; Vol. 13, No. 2, pp. 127-137.
Tost, J., Gut, I. Analysis of gene-specific DNA methylation patterns by pyrosequencing technology. Methods Mol Biol. 2007; 373:89-102.
Tryka K A, et al. (2014) NCBI's Database of Genotypes and Phenotypes: dbGaP. Nucleic acids research 42(Database issue):D975-979.
Van Allen E M, et al. (2015) Genomic correlates of response to CTLA-4 blockade in metastatic melanoma. Science 350(6257):207-211.
Van der Gun et al. (2010), Targeted DNA methylation by a DNA methyltransferase coupled to a triple helix forming oligonucleotide to down-regulate the epithelial cell adhesion molecule, Bioconjugate Chem; 21:1239-1245.
Warnecke, P., Stirzaker, C., Song, J., Grunau, C., Melki, J., Clark, S. Identification and resolution of artifacts in bisulfite sequencing. Methods, 2002; 27:101-7.
Xiong, Z., Laird, P. COBRA: a sensitive and quantitative DNA methylation assay. Nucleic Acids Res. 1997; 25:2532-4.
Yoshihama S, et al. (2016) NLRC5/MHC class I transactivator is a target for immune evasion in cancer. Proceedings of the National Academy of Sciences of the United States of America.
Yoshihama S, Vijayan S, Sidiq T, & Kobayashi K S (2017) NLRC5/CITA: A Key Player in Cancer Immune Surveillance. Trends Cancer 3(1):28-38.

We claim:

1. A method of identifying a subject as having a cancer that is likely or not likely to evade the immune system of the subject and treating the subject, the method comprising the steps of:
(a) determining the amount of NLRC5 mRNA or NLRC5 protein and, optionally, neoantigen load, mutation number, or expression of genes involved in immune responses, including but not limited to CTLA4, PD1, PD-L1 and PD-L2 in a subject with higher levels of those parameters in:
i) a test sample obtained from the subject, and
ii) optionally, a control sample; and
(b) optionally, obtaining one or more reference values for the amount of NLRC5 mRNA or NLRC5 protein and, optionally, neoantigen load, mutation number, or expression of genes involved in immune responses, including but not limited to CTLA4, PD1, PD-L1 and PD-L2 in a subject with higher levels of those parameters, and
(i) identifying the subject as having a cancer that is likely to evade the immune system of the subject based on the amount of NLRC5 mRNA or NLRC5 protein and, optionally neoantigen load, mutation number, or expression of genes involved in immune responses, including but not limited to CTLA4, PD1, PD-L1 and PD-L2 in a subject with higher levels of those parameters in the test sample as compared to the control sample or the reference value and administering a first therapy and/or a second therapy to the subject to treat the cancer, or
(ii) identifying the subject as having a cancer that is not likely to evade the immune system of the subject based on the amount of NLRC5 mRNA or NLRC5 protein and, optionally, neoantigen load, mutation number, or expression of genes involved in immune responses, including but not limited to CTLA4, PD1, PD-L1 and PD-L2 in a subject with higher levels of those parameters, in the test sample as compared to the control sample or the reference value and administering a first therapy to the subject to treat the cancer and/or withholding the administration of the second therapy to the subject.

2. The method of claim 1, wherein the step of identifying the subject as having a cancer that is likely or not likely to evade the immune system of the subject comprises:
i) identifying the subject as having a cancer that is likely to evade the immune system of the subject if the amount of NLRC5 mRNA or NLRC5 protein and, optionally, neoantigen load, mutation number, or expression of genes involved in immune responses, including but not limited to CTLA4, PD1, PD-L1 and PD-L2 in a subject with higher levels of those parameters, if determined, in the test sample is lower than the amount of NLRC5 mRNA or NLRC5 protein and, optionally, neoantigen load, mutation number, or expression of genes involved in immune responses, including but not limited to CTLA4, PD1, PD-L1 and PD-L2 in a subject with higher levels of those parameters, if determined, in the control sample or reference value, or
ii) identifying the subject as having a cancer that is not likely to evade the immune system of the subject if the amount of NLRC5 mRNA or NLRC5 protein and, optionally, neoantigen load, mutation number, or expression of genes involved in immune responses, including but not limited to CTLA4, PD1, PD-L1 and PD-L2 in a subject with higher levels of those parameters in the test sample is equal to or higher than the amount of NLRC5 mRNA or NLRC5 protein in the control sample or reference value.

3. A method of identifying a subject as having a cancer that is likely or not likely to evade the immune system of the subject and treating the subject, the method comprising the steps of:
(a) determining the transcription factor activity of NLRC5 protein and, optionally, neoantigen load, mutation number, or expression of genes involved in immune responses, including but not limited to CTLA4, PD1, PD-L1 and PD-L2 in a subject with higher levels of those parameters in:
i) a test sample obtained from the subject, and
ii) optionally, a control sample; and
(b) optionally, obtaining one or more reference values for the transcription factor activity of NLRC5 protein and, optionally, neoantigen load, mutation number, or expression of genes involved in immune responses, including but not limited to CTLA4, PD1, PD-L1 and PD-L2 in a subject with higher levels of those parameters, and
(i) identifying the subject as having a cancer that is likely to evade the immune system of the subject based on the transcription factor activity of NLRC5 protein and, optionally, neoantigen load, mutation number, or expression of genes involved in immune responses, including but not limited to CTLA4, PD1, PD-L1 and PD-L2 in a subject with higher levels of those parameters, if determined, in the test sample as compared to the control sample or the reference value and administering a first therapy and/or a second therapy to the subject to treat the cancer, or
(ii) identifying the subject as having a cancer that is not likely to evade the immune system of the subject based on the transcription factor activity of NLRC5 protein and, optionally, neoantigen load, mutation number, or expression of genes involved in immune responses, including but not limited to CTLA4, PD1, PD-L1 and PD-L2 in a subject with higher levels of those parameters, if determined, in the test sample as compared to the control sample or the reference value and administering a first therapy to the subject to treat the cancer and/or withholding the administration of the second therapy to the subject.

4. The method of claim 3, wherein the step of identifying the subject as having a cancer that is likely or not likely to evade the immune system comprises:
i) identifying the subject as having a cancer that is likely to evade the immune system of the subject if the transcription factor activity of NLRC5 protein and the level of neoantigen load, mutation number, or expression of genes involved in immune responses, including but not limited to CTLA4, PD1, PD-L1 and PD-L2 in a subject with higher levels of those parameters, if determined, in the test sample is lower than the transcription factor activity of NLRC5 protein or neoantigen load, mutation number, or expression of genes involved in immune responses, including but not limited to CTLA4, PD1, PD-L1 and PD-L2 in a subject with higher levels of those parameters in the control sample or reference value, if determined, or
ii) identifying the subject as having a cancer that is not likely to evade the immune system of the subject if the transcription factor activity of NLRC5 protein and, optionally, neoantigen load, mutation number, or expression of genes involved in immune responses, including but not limited to CTLA4, PD1, PD-L1 and PD-L2 in a subject with higher levels of those parameters, if determined, in the test sample is equal to or higher than the transcription factor activity of NLRC5 protein in the control sample or reference value.

5. A method of identifying a subject as having a cancer that is likely or not likely to evade the immune system of the subject and treating the subject, the method comprising the steps of:
(a) determining the sequence of the protein coding region of nlrc5 or a portion thereof and, optionally, neoantigen load, mutation number, or expression of genes involved in immune responses, including but not limited to CTLA4, PD1, PD-L1 and PD-L2 in a subject with higher levels of those parameters, in a test sample obtained from the subject; and
(b) optionally, determining the sequence of NLRC5 protein encoded by nlrc5 or a portion thereof in the test sample and, optionally, determining the activity of a wild-type NLRC5 protein and the NLRC5 protein encoded by the nlrc5 in the test sample, and
(i) identifying the subject as having a cancer that is likely to evade the immune system of the subject if the NLRC5 protein in the test sample contains a mutation that reduces the transcription factor activity of NLRC5 protein and, optionally, low neoantigen load, mutation number, or low expression of genes involved in immune responses, including but not limited to CTLA4, PD1, PD-L1 and PD-L2 in a subject with higher levels of those parameters, if determined, in comparison to the test sample and/or neoantigen reference value as compared to the wild-type NLRC5 protein and, optionally, administering a first therapy and/or a second therapy to the subject to treat the cancer, or
(ii) identifying the subject as having a cancer that is not likely to evade the immune system of the subject if the NLRC5 protein in the test sample does not contain a mutation or contains a mutation that does not affect or increases the transcription factor activity of NLRC5 protein and neoantigen load, mutation number, or expression of genes involved in immune responses, including but not limited to CTLA4, PD1, PD-L1 and PD-L2 in a subject with higher levels of those parameters, if determined, is elevated in the test sample as compared to the wild-type NLRC5 protein and control and/or reference of those variables and, optionally, administering a first therapy to the subject to treat the cancer and/or withholding the administration of the second therapy to the subject.

6. The method of claim 5, wherein a subject having one or more of the following mutations in the NLRC5 protein as compared to the wild-type NLRC5 protein is identified as having a cancer that is likely to evade the immune system of the subject: L181P, R262C, R550W, A737D, H1717fs*29, R1830C, and Q1847*.

7. The method of claim 5, wherein a subject having one or more of the following mutations in the NLRC5 protein as compared to the wild-type NLRC5 protein is identified as having a cancer that is not likely to evade the immune system of the subject: R386W, S496F, R574H, D884N, T1173M, and A1512T.

8. A method of identifying a subject as having a cancer that is likely or not likely to evade the immune system of the subject, and treating the subject, the method comprising the steps of:
(a) determining the level of methylation of nlrc5 or a portion thereof and, optionally, neoantigen load, mutation number, or expression of genes involved in immune responses, including but not limited to CTLA4, PD1, PD-L1 and PD-L2 in a subject with higher levels of those parameters in:
i) a test sample obtained from the subject, and
ii) optionally, a control sample; and
(b) optionally, obtaining one or more reference values for the levels of methylation of nlrc5 or a portion thereof and, optionally, neoantigen load, mutation number, or expression of genes involved in immune responses, including but not limited to CTLA4, PD1, PD-L1 and PD-L2 in a subject with higher levels of those parameters, and
(i) identifying the subject as having a cancer that is likely to evade the immune system of the subject based on the level of methylation of nlrc5 or a portion thereof in the test sample as compared to the control sample or the reference value and neoantigen load, mutation number, or expression of genes involved in immune responses, including but not limited to CTLA4, PD1, PD-L1 and PD-L2 in a subject with higher levels of those parameters, in comparison to a control sample or reference value, if determined, optionally, administering a first therapy and/or a second therapy to the subject to treat the cancer, or
(ii) identifying the subject as having a cancer that is not likely to evade the immune system of the subject based on the level of methylation of nlrc5 or a portion thereof and neoantigen load, mutation number, or expression of genes involved in immune responses, including but not limited to CTLA4, PD1, PD-L1 and PD-L2 in a subject with higher levels of those parameters, if determined in the test sample as compared to the control sample or the reference value and, optionally, administering a first therapy to the subject to treat the cancer and/or withholding the administration of the second therapy to the subject.

9. The method of claim 8, wherein the step of identifying the subject as having a cancer that is likely or not likely to evade the immune system comprises:
i) identifying the subject as having a cancer that is likely to evade the immune system of the subject if the level of methylation of nlrc5 or a portion thereof in the test sample is higher than the level of methylation of nlrc5 or a portion thereof in the control sample and neoantigen load, mutation number, or expression of genes involved in immune responses, including but not limited to CTLA4, PD1, PD-L1 and PD-L2 in a subject with higher levels of those parameters, if determined are lower in the test sample in comparison to a control sample or reference value, or
ii) identifying the subject as having a cancer that is not likely to evade the immune system of the subject if the level of methylation of nlrc5 or a portion thereof in the test sample is equal to or lower than the level methylation of nlrc5 or a portion thereof in the control sample and neoantigen load, mutation number, or expression of genes involved in immune responses, including but not limited to CTLA4, PD1, PD-L1 and PD-L2 in a subject with higher levels of those parameters, if determined, are elevated in comparison to a test sample or reference value.

10. A method of identifying a subject as having a cancer that is likely or not likely to evade the immune system of the subject, and treating the subject, the method comprising the steps of:
(a) determining the beta-value for methylation of nlrc5 or a portion thereof and, optionally, neoantigen load, mutation number, or expression of genes involved in immune responses, including but not limited to CTLA4, PD1, PD-L1 and PD-L2 in a subject with higher levels of those parameters in a test sample obtained from the subject, and
(b) identifying the subject as:
i) having a cancer that is likely to evade the immune system of the subject if the beta-value for methylation of nlrc5 or a portion thereof in the test sample is above 0.2, 0.3 or 0.4, and neoantigen load, mutation number, or expression of genes involved in immune responses, including but not limited to CTLA4, PD1, PD-L1 and PD-L2 in a subject with higher levels of those parameters is low in comparison to the control sample or reference value, if determined or
b) having a cancer that is not likely to evade the immune system of the subject if the beta-value for methylation of nlrc5 or a portion thereof in the test sample is below 0.2, 0.3 or 0.4 and neoantigen load, mutation number, or expression of genes involved in immune responses, including but not limited to CTLA4, PD1, PD-L1 and PD-L2 in a subject with higher levels of those parameters is elevated in comparison to the control sample or reference value, if determined.

11. The method of claim 8, wherein the portion of nlrc5 has the sequence of SEQ ID NO: 4, 5 or 6.

12. A method of identifying a subject as having a cancer that is likely or not likely to evade the immune system of the subject, and, treating the subject, the method comprising the steps of:
(a) determining the copy number of nlrc5 in a test sample obtained from the subject, and
(i) identifying the subject as having a cancer that is likely to evade the immune system of the subject based on the copy number for nlrc5 being below about two in the test sample and, optionally, low neoantigen load, mutation number, or expression of genes involved in immune responses, including but not limited to CTLA4, PD1, PD-L1 and PD-L2 in a subject with higher levels of those parameters in comparison to a control sample or reference value administering a first therapy and/or a second therapy to the subject to treat the cancer, or
(ii) identifying the subject as having a cancer that is not likely to evade the immune system of the subject based on the copy number for nlrc5 being above about two in the test sample and optionally, elevated neoantigen load, mutation number, or expression of genes involved in immune responses, including but not limited to CTLA4, PD1, PD-L1 and PD-L2 in a subject with higher levels of those parameters in comparison to a control sample or reference value and administering a first therapy to the subject to treat the cancer and/or withholding the administration of the second therapy to the subject.

13. The method of claim 1, wherein the first therapy is a non-immunotherapeutic treatment or an immunotherapy, wherein the non-immunotherapeutic treatment or the immunotherapy is designed to kill and/or control the proliferation of cancer cells and the second therapy is designed to reduce the ability of the cancer cells to evade the immune system of the subject and is directed to activating the MHC class I transactivation pathway by activating the expression of nlrc5 or the expression and/or activity of NLRC5 mRNA or protein in the cancer cells.

14. The method of claim 13, wherein the immunotherapy comprises:
i) administering to the subject an agent that blocks a protein that inhibits the strength and duration of the immune response in the subject,
ii) adoptive cell transfer,
iii) administering to the subject a therapeutic antibody that causes the immune system-mediated destruction of the cancer cells,
iv) administering to the subject a non-antibody immune system molecule that causes the immune system-mediated destruction of the cancer cells,
v) administering to the subject a cancer vaccine, or
vi) administering to the subject an immune system modulator.

15. The method of claim 13, wherein the second therapy comprises administering to the subject:
a) an agent that causes the activation of NLRC5 protein activity;
b) a wild-type or mutant NLRC5 protein or a nucleotide encoding the wild-type or mutant NLRC5 protein;
c) an agent that causes a non-specific demethylation of genomic DNA; or
d) an agent that causes a site-specific demethylation of nlrc5 or a portion thereof.

16. The method of claim 13, wherein the first therapy is the non-immunotherapeutic treatment and the second therapy is not administered to the subject identified as having a cancer that is likely or not likely to evade the immune system of the subject.

17. The method of claim 13, wherein the first therapy is the non-immunotherapeutic treatment and the second therapy is not administered to the subject identified as having a cancer that is not likely to evade the immune system of the subject.

18. The method of claim 13, wherein the first therapy is the non-immunotherapeutic treatment and the second therapy is administered to the subject identified as having a cancer that is likely to evade the immune system of the subject.

19. The method of claim 13, wherein the first therapy is the immunotherapy and the second therapy is not administered to the subject identified as having a cancer that is not likely to evade the immune system of the subject.

20. The method of claim 13, wherein the first therapy is the immunotherapy and the second therapy is administered to the subject identified as having a cancer that is likely to evade the immune system of the subject.

21. The method of claim 13, wherein the first and second therapies are administered simultaneously or separately to the subject.

22. The method of claim 13, wherein the first therapy and/or the second therapy are administered in a sub-therapeutic amount.


SEQUENCE LISTING (34 sequences)

SEQ ID NO: 1 - DNA, 5601 bp, Homo sapiens
atggaccccgttggcctccagctcggcaacaagaacctgtggagctgtcttgtgaggctgctcaccaaagacccagaatggctgaacgccaagatgaagttcttcctccccaacacggacctggattccaggaacgagaccttggaccctgaacagagagtcatcctgcaactcaacaagctgcatgtccagggttcggacacctggcagtctttcattcattgtgtgtgcatgcagctggaggtgcctctggacctggaggtgctgctgctgagtacttttggctatgatgatgggttcaccagccagctgggagctgaggggaaaagccaacctgaatctcagctccaccatggcctgaagcgcccacatcagagctgtgggtcctcaccccgccggaagcagtgcaagaagcagcagctagagttggccaagaagtacctgcagctcctgcggacctctgcccagcagcgctacaggagccaaatccctgggtcagggcagccccacgccttccaccaggtctatgtccctccaatcctgcgccgggccacagcatccttagacactccggagggggccattatgggggacgtcaaggtggaagatggtgctgacgtgagcatctcggacctcttcaacaccagggttaacaagggcccgagggtgaccgtgcttttggggaaggctggcatgggcaagaccacgctggcccaccggctctgccagaagtgggcagagggccatctgaactgtttccaggccctgttcctttttgaattccgccagctcaacttgatcacgaggttcctgacaccgtccgagctcctttttgatctgtacctgagccctgaatcggaccacgacactgtcttccagtacctggagaagaacgctgaccaagtcctgctgatctttgatgggctagatgaggccctccagcctatgggtcctgatggcccaggcccagtcctcacccttttctcccatctctgcaatgggaccctcctgcctggctgccgggtgatggctacctcccgtccagggaagctgcctgcctgcctgcctgcagaggcagccatggtccacatgttgggctttgatgggccacgggtggaagaatatgtgaatcacttcttcagcgcccagccatcgcgggagggggccctggtggagttacagacaaatggacgtctccgaagcctgtgtgcggtgcccgcactgtgccaagtcgcctgtctctgcctccaccatctgcttcctgaccacgccccaggccagtctgtggccctcctgcccaacatgactcagctctatatgcagatggtgctcgccctcagcccccctgggcacttgcccacctcgtccctactggacctgggggaggtggccctgaggggcctggagacagggaaggttatcttctatgcaaaagatattgctccacccttgatagcttttggggccactcacagcctgctgacttccttctgcgtctgcacaggccctgggcaccagcagacaggctatgctttcacccacctcagcctgcaggagtttcttgctgccctgcacctgatggccagccccaaggtgaacaaagacacacttacccagtatgttaccctccattcccgctgggtacagcggaccaaagctagactgggcctctcagaccacctccccaccttcctggcgggcctggcatcctgcacctgccgccccttccttagccacctggcgcagggcaatgaggactgtgtgggtgccaagcaggctgctgtagtgcaggtgttgaagaagttggccacccgcaagctcacagggccaaaggttgtagagctgtgtcactgtgtggatgagacacaggagcctgagctggccagtctcaccgcacaaagcctcccctatcaactgcccttccacaatttcccactgacctgcaccgacctggccaccctgaccaacatcctagagcacagggaggcccccatccacctggattttgatggctgtcccctggagccccactgccctgaggctctggtaggctgtgggcagatagagaatctcagctttaagagcaggaagtgtggggatgcctttgcagaagccctctccaggagcttgccgacaatggggaggctgcagatgctggggttagcaggaagtaaaatcactgcccgaggcatcagccacctggtgaaagctttgcctctctgtccacagctgaaagaagtcagttttcgggacaaccagctcagtgaccaggtggtgctgaacattgtggaggttctccctcacctaccacggctccggaagcttgacctgagcagcaacagcatctgcgtgtcaaccctactctgcttggcaagggtggcagtcacgtgtcctaccgtcaggatgcttcaggccagggaggcggacctcatcttccttctttccccgcccacagagacaactgcagagctacaaagagctccagacctgcaggaaagtgacggccagaggaaaggggctcagagcagaagcttgacgctcaggctgcagaagtgtcagctccaggtccacgatgcggaggccctcatagccctgctccaggaaggccctcacctggaggaagtggacctctcagggaaccagctggaagatgaaggctgtcggctgatggcagaggctgcatcccagctgcacatcgccaggaagctggacctcagtaacaacgggctttctgtggccggggtgcattgtgtgctgagggccgtgagtgcgtgctggaccctggcagagctgcacatcagcctgcagcacaaaactgtgatcttcatgtttgcccaggagccagaggagcagaaggggccccaggagagggctgcatttcttgacagcctcatgctccagatgccctctgagctgcctctgagctcccgaaggatgaggctgacacattgtggcctccaagaaaagcacctagagcagctctgcaaggctctgggaggaagctgccacctcggtcacctccacctcgacttctcaggcaatgctctgggggatgaaggtgcagcccggctggctcagctgctcccagggctgggagctctgcagtccttgaacctcagtgagaacggtttgtccctggatgccgtgttgggtttggttcggtgcttctccactctgcagtggctcttccgcttggacatcagctttgaaagccaacacatcctcctgagaggggacaagacaagcagggatatgtgggccactggatctttgccagacttcccagctgcagccaagttcttagggttccgtcagcgctgcatccccaggagcctctgcctcagtgagtgtcctctggagcccccaagcctcacccgcctctgtgccactctgaaggactgcccgggacccctggaactgcaattgtcctgtgagttcctgagtgaccagagcctggagactctactggactgcttacctcaactccctcagctgagcctgctgcagctgagccagacgggactgtccccgaaaagccccttcctgctggccaacaccttaagcctgtgtccacgggttaaaaaggtggatctcaggtccctgcaccatgcaactttgcacttcagatccaacgaggaggaggaaggcgtgtgctgtggcaggttcacaggctgcagcctcagccaggagcacgtagagtcactctgctggttgctgagcaagtgtaaagacctcagccaggtggatctctcagcaaacctgctgggcgacagcggactcagatgccttctggaatgtctgccgcaggtgcccatctccggtttgcttgatctgagtcacaacagcatttctcaggaaagtgccctgtacctgctggagacactgccctcctgcccacgtgtccgggaggcctcagtgaacctgggctctgagcagagcttccggattcacttctccagagaggaccaggctgggaagacactcaggctaagtgagtgcagcttccggccagagcacgtgtccaggctggccaccggcttgagcaagtccctgcagctgacggagctcacgctgacccagtgctgcctgggccagaagcagctggccatcctcctgagcttggtggggcgacccgcagggctgttcagcctcagggtgcaggagccgtgggcggacagagccagggttctctccctgttagaagtctgcgcccaggcctcaggcagtgtcactgaaatcagcatctccgagacccagcagcagctctgtgtccagctggaatttcctcgccaggaagagaatccagaagctgtggcactcaggttggctcactgtgaccttggagcccaccacagccttcttgtcgggcagctgatggagacatgtgccaggctgcagcagctcagcttgtctcaggttaacctctgtgaggacgatgatgccagttccctgctgctgcagagcctcctgctgtccctctctgagctgaagacatttcggctgacctccagctgtgtgagcaccgagggcctcgcccacctggcatctggtctgggccactgccaccacttggaggagctggacttgtctaacaatcaatttgatgaggagggcaccaaggcgctgatgagggcccttgaggggaaatggatgctaaagaggctggacctcagtcaccttctgctgaacagctccaccttggccttgcttactcacagactaagccagatgacctgcctgcagagcctcagactgaacaggaacagtatcggtgatgtcggttgctgccacctttctgaggctctcagggctgccaccagcctagaggagctggacttgagccacaaccagattggagacgctggtgtccagcacttagctaccatcctgcctgggctgccagagctcaggaagatagacctctcagggaatagcatcagctcagccgggggagtgcagttggcagagtctctcgttctttgcaggcgcctggaggagttgatgcttggctgcaatgccctgggggatcccacagccctggggctggctcaggagctgccccagcacctgagggtcctacacctaccattcagccatctgggcccaggtggggccctgagcctggcccaggccctggatggatccccccatttggaagagatcagcttggcggaaaacaacctggctggaggggtcctgcgtttctgtatggagctcccgctgctcagacagatagacctggtttcctgtaagattgacaaccagactgccaagctcctcacctccagcttcacgagctgccctgccctggaagtaatcttgctgtcctggaatctcctcggggatgaggcagctgccgagctggcccaggtgctgccgcagatgggccggctgaagagagtggacctggagaagaatcagatcacagctttgggggcctggctcctggctgaaggactggcccaggggtctagcatccaagtcatccgcctctggaataaccccattccctgcgacatggcccagcacctgaagagccaggagcccaggctggactttgccttctttgacaaccagccccaggccccttggggtacttga

SEQ ID NO: 2 - protein, 1866 aa, Homo sapiens
MDPVGLQLGNKNLWSCLVRLLTKDPEWLNAKMKFFLPNTDLDSRNETLDPEQRVILQLNKLHVQGSDTWQSFIHCVCMQLEVPLDLEVLLLSTFGYDDGFTSQLGAEGKSQPESQLHHGLKRPHQSCGSSPRRKQCKKQQLELAKKYLQLLRTSAQQRYRSQIPGSGQPHAFHQVYVPPILRRATASLDTPEGAIMGDVKVEDGADVSISDLFNTRVNKGPRVTVLLGKAGMGKTTLAHRLCQKWAEGHLNCFQALFLFEFRQLNLITRFLTPSELLFDLYLSPESDHDTVFQYLEKNADQVLLIFDGLDEALQPMGPDGPGPVLTLFSHLCNGTLLPGCRVMATSRPGKLPACLPAEAAMVHMLGFDGPRVEEYVNHFFSAQPSREGALVELQTNGRLRSLCAVPALCQVACLCLHHLLPDHAPGQSVALLPNMTQLYMQMVLALSPPGHLPTSSLLDLGEVALRGLETGKVIFYAKDIAPPLIAFGATHSLLTSFCVCTGPGHQQTGYAFTHLSLQEFLAALHLMASPKVNKDTLTQYVTLHSRWVQRTKARLGLSDHLPTFLAGLASCTCRPFLSHLAQGNEDCVGAKQAAVVQVLKKLATRKLTGPKVVELCHCVDETQEPELASLTAQSLPYQLPFHNFPLTCTDLATLTNILEHREAPIHLDFDGCPLEPHCPEALVGCGQIENLSFKSRKCGDAFAEALSRSLPTMGRLQMLGLAGSKITARGISHLVKALPLCPQLKEVSFRDNQLSDQVVLNIVEVLPHLPRLRKLDLSSNSICVSTLLCLARVAVTCPTVRMLQAREADLIFLLSPPTETTAELQRAPDLQESDGQRKGAQSRSLTLRLQKCQLQVHDAEALIALLQEGPHLEEVDLSGNQLEDEGCRLMAEAASQLHIARKLDLSNNGLSVAGVHCVLRAVSACWTLAELHISLQHKTVIFMFAQEPEEQKGPQERAAFLDSLMLQMPSELPLSSRRMRLTHCGLQEKHLEQLCKALGGSCHLGHLHLDFSGNALGDEGAARLAQLLPGLGALQSLNLSENGLSLDAVLGLVRCFSTLQWLFRLDISFESQHILLRGDKTSRDMWATGSLPDFPAAAKFLGFRQRCIPRSLCLSECPLEPPSLTRLCATLKDCPGPLELQLSCEFLSDQSLETLLDCLPQLPQLSLLQLSQTGLSPKSPFLLANTLSLCPRVKKVDLRSLHHATLHFRSNEEEEGVCCGRFTGCSLSQEHVESLCWLLSKCKDLSQVDLSANLLGDSGLRCLLECLPQVPISGLLDLSHNSISQESALYLLETLPSCPRVREASVNLGSEQSFRIHFSREDQAGKTLRLSECSFRPEHVSRLATGLSKSLQLTELTLTQCCLGQKQLAILLSLVGRPAGLFSLRVQEPWADRARVLSLLEVCAQASGSVTEISISETQQQLCVQLEFPRQEENPEAVALRLAHCDLGAHHSLLVGQLMETCARLQQLSLSQVNLCEDDDASSLLLQSLLLSLSELKTFRLTSSCVSTEGLAHLASGLGHCHHLEELDLSNNQFDEEGTKALMRALEGKWMLKRLDLSHLLLNSSTLALLTHRLSQMTCLQSLRLNRNSIGDVGCCHLSEALRAATSLEELDLSHNQIGDAGVQHLATILPGLPELRKIDLSGNSISSAGGVQLAESLVLCRRLEELMLGCNALGDPTALGLAQELPQHLRVLHLPFSHLGPGGALSLAQALDGSPHLEEISLAENNLAGGVLRFCMELPLLRQIDLVSCKIDNQTAKLLTSSFTSCPALEVILLSWNLLGDEAAAELAQVLPQMGRLKRVDLEKNQITALGAWLLAEGLAQGSSIQVIRLWNNPIPCDMAQHLKSQEPRLDFAFFDNQPQAPWGT

SEQ ID NO: 3 - DNA, 6742 bp, Homo sapiens
gagtctgcactatggaaacaacctgtcaatccagctcaaggcacacatagcccagacacccatgagaccctctccgtggggaccctagagcacctatcatgaacgaggagaccaaggctggctcctcatggaccccgttggcctccagctcggcaacaagaacctgtggagctgtcttgtgaggctgctcaccaaagacccagaatggctgaacgccaagatgaagttcttcctccccaacacggacctggattccaggaacgagaccttggaccctgaacagagagtcatcctgcaactcaacaagctgcatgtccagggttcggacacctggcagtctttcattcattgtgtgtgcatgcagctggaggtgcctctggacctggaggtgctgctgctgagtacttttggctatgatgatgggttcaccagccagctgggagctgaggggaaaagccaacctgaatctcagctccaccatggcctgaagcgcccacatcagagctgtgggtcctcaccccgccggaagcagtgcaagaagcagcagctagagttggccaagaagtacctgcagctcctgcggacctctgcccagcagcgctacaggagccaaatccctgggtcagggcagccccacgccttccaccaggtctatgtccctccaatcctgcgccgggccacagcatccttagacactccggagggggccattatgggggacgtcaaggtggaagatggtgctgacgtgagcatctcggacctcttcaacaccagggttaacaagggcccgagggtgaccgtgcttttggggaaggctggcatgggcaagaccacgctggcccaccggctctgccagaagtgggcagagggccatctgaactgtttccaggccctgttcctttttgaattccgccagctcaacttgatcacgaggttcctgacaccgtccgagctcctttttgatctgtacctgagccctgaatcggaccacgacactgtcttccagtacctggagaagaacgctgaccaagtcctgctgatctttgatgggctagatgaggccctccagcctatgggtcctgatggcccaggcccagtcctcacccttttctcccatctctgcaatgggaccctcctgcctggctgccgggtgatggctacctcccgtccagggaagctgcctgcctgcctgcctgcagaggcagccatggtccacatgttgggctttgatgggccacgggtggaagaatatgtgaatcacttcttcagcgcccagccatcgcgggagggggccctggtggagttacagacaaatggacgtctccgaagcctgtgtgcggtgcccgcactgtgccaagtcgcctgtctctgcctccaccatctgcttcctgaccacgccccaggccagtctgtggccctcctgcccaacatgactcagctctatatgcagatggtgctcgccctcagcccccctgggcacttgcccacctcgtccctactggacctgggggaggtggccctgaggggcctggagacagggaaggttatcttctatgcaaaagatattgctccacccttgatagcttttggggccactcacagcctgctgacttccttctgcgtctgcacaggccctgggcaccagcagacaggctatgctttcacccacctcagcctgcaggagtttcttgctgccctgcacctgatggccagccccaaggtgaacaaagacacacttacccagtatgttaccctccattcccgctgggtacagcggaccaaagctagactgggcctctcagaccacctccccaccttcctggcgggcctggcatcctgcacctgccgccccttccttagccacctggcgcagggcaatgaggactgtgtgggtgccaagcaggctgctgtagtgcaggtgttgaagaagttggccacccgcaagctcacagggccaaaggttgtagagctgtgtcactgtgtggatgagacacaggagcctgagctggccagtctcaccgcacaaagcctcccctatcaactgcccttccacaatttcccactgacctgcaccgacctggccaccctgaccaacatcctagagcacagggaggcccccatccacctggattttgatggctgtcccctggagccccactgccctgaggctctggtaggctgtgggcagatagagaatctcagctttaagagcaggaagtgtggggatgcctttgcagaagccctctccaggagcttgccgacaatggggaggctgcagatgctggggttagcaggaagtaaaatcactgcccgaggcatcagccacctggtgaaagctttgcctctctgtccacagctgaaagaagtcagttttcgggacaaccagctcagtgaccaggtggtgctgaacattgtggaggttctccctcacctaccacggctccggaagcttgacctgagcagcaacagcatctgcgtgtcaaccctactctgcttggcaagggtggcagtcacgtgtcctaccgtcaggatgcttcaggccagggaggcggacctcatcttccttctttccccgcccacagagacaactgcagagctacaaagagctccagacctgcaggaaagtgacggccagaggaaaggggctcagagcagaagcttgacgctcaggctgcagaagtgtcagctccaggtccacgatgcggaggccctcatagccctgctccaggaaggccctcacctggaggaagtggacctctcagggaaccagctggaagatgaaggctgtcggctgatggcagaggctgcatcccagctgcacatcgccaggaagctggacctcagtaacaacgggctttctgtggccggggtgcattgtgtgctgagggccgtgagtgcgtgctggaccctggcagagctgcacatcagcctgcagcacaaaactgtgatcttcatgtttgcccaggagccagaggagcagaaggggccccaggagagggctgcatttcttgacagcctcatgctccagatgccctctgagctgcctctgagctcccgaaggatgaggctgacacattgtggcctccaagaaaagcacctagagcagctctgcaaggctctgggaggaagctgccacctcggtcacctccacctcgacttctcaggcaatgctctgggggatgaaggtgcagcccggctggctcagctgctcccagggctgggagctctgcagtccttgaacctcagtgagaacggtttgtccctggatgccgtgttgggtttggttcggtgcttctccactctgcagtggctcttccgcttggacatcagctttgaaagccaacacatcctcctgagaggggacaagacaagcagggatatgtgggccactggatctttgccagacttcccagctgcagccaagttcttagggttccgtcagcgctgcatccccaggagcctctgcctcagtgagtgtcctctggagcccccaagcctcacccgcctctgtgccactctgaaggactgcccgggacccctggaactgcaattgtcctgtgagttcctgagtgaccagagcctggagactctactggactgcttacctcaactccctcagctgagcctgctgcagctgagccagacgggactgtccccgaaaagccccttcctgctggccaacaccttaagcctgtgtccacgggttaaaaaggtggatctcaggtccctgcaccatgcaactttgcacttcagatccaacgaggaggaggaaggcgtgtgctgtggcaggttcacaggctgcagcctcagccaggagcacgtagagtcactctgctggttgctgagcaagtgtaaagacctcagccaggtggatctctcagcaaacctgctgggcgacagcggactcagatgccttctggaatgtctgccgcaggtgcccatctccggtttgcttgatctgagtcacaacagcatttctcaggaaagtgccctgtacctgctggagacactgccctcctgcccacgtgtccgggaggcctcagtgaacctgggctctgagcagagcttccggattcacttctccagagaggaccaggctgggaagacactcaggctaagtgagtgcagcttccggccagagcacgtgtccaggctggccaccggcttgagcaagtccctgcagctgacggagctcacgctgacccagtgctgcctgggccagaagcagctggccatcctcctgagcttggtggggcgacccgcagggctgttcagcctcagggtgcaggagccgtgggcggacagagccagggttctctccctgttagaagtctgcgcccaggcctcaggcagtgtcactgaaatcagcatctccgagacccagcagcagctctgtgtccagctggaatttcctcgccaggaagagaatccagaagctgtggcactcaggttggctcactgtgaccttggagcccaccacagccttcttgtcgggcagctgatggagacatgtgccaggctgcagcagctcagcttgtctcaggttaacctctgtgaggacgatgatgccagttccctgctgctgcagagcctcctgctgtccctctctgagctgaagacatttcggctgacctccagctgtgtgagcaccgagggcctcgcccacctggcatctggtctgggccactgccaccacttggaggagctggacttgtctaacaatcaatttgatgaggagggcaccaaggcgctgatgagggcccttgaggggaaatggatgctaaagaggctggacctcagtcaccttctgctgaacagctccaccttggccttgcttactcacagactaagccagatgacctgcctgcagagcctcagactgaacaggaacagtatcggtgatgtcggttgctgccacctttctgaggctctcagggctgccaccagcctagaggagctggacttgagccacaaccagattggagacgctggtgtccagcacttagctaccatcctgcctgggctgccagagctcaggaagatagacctctcagggaatagcatcagctcagccgggggagtgcagttggcagagtctctcgttctttgcaggcgcctggaggagttgatgcttggctgcaatgccctgggggatcccacagccctggggctggctcaggagctgccccagcacctgagggtcctacacctaccattcagccatctgggcccaggtggggccctgagcctggcccaggccctggatggatccccccatttggaagagatcagcttggcggaaaacaacctggctggaggggtcctgcgtttctgtatggagctcccgctgctcagacagatagacctggtttcctgtaagattgacaaccagactgccaagctcctcacctccagcttcacgagctgccctgccctggaagtaatcttgctgtcctggaatctcctcggggatgaggcagctgccgagctggcccaggtgctgccgcagatgggccggctgaagagagtggacctggagaagaatcagatcacagctttgggggcctggctcctggctgaaggactggcccaggggtctagcatccaagtcatccgcctctggaataaccccattccctgcgacatggcccagcacctgaagagccaggagcccaggctggactttgccttctttgacaaccagccccaggccccttggggtacttgatggccccctcaagacctttggaatccagccaagtgatgcacccaaatgatccacctttcgcccactgggataattgactcaggaaagaagagcctcggcagggcgctctgcactccacccaggaggaaggatacgtgtgtcctgctgcagtcctcagggagaacttttttgggaaccaggagctgggtctggacaaaggagtaccctgcattacgtgggatatgtgtgatcaattggggacatgcgacacacaatgagggtgtcatgacaatgcatgacacgtacggttatatgtggcagtgtgaccccttgacatgtggcgttacatgaaagtcagtgtggcacgtgttctgtggcatgggtgctggcatcccaagtagcaggatacatgattgttggtctatatatgacacatgacaaatgtccatgtcacaggactcatggctggccagatgacctcaggctggcccaagatctaatttattaatttttaaagcaaatacatatttatagattgtgtgtatggagcagctaagtcaggaaaagtcttccgcccgagctgggaggggagagtgtccatgcactgaccagtccaggggctcaagggccagggctctggaacaagccagggactcagccattaagtcccctcctgcctcaatcctcagcctacccatctataaacttgatgactcctcccttacttacatactagcttccaaggacaggtggaggtagggccagcctggcgggagtggagaagcccagtctgtcctatgtaagggacaaagccaggtctaatggtactgggtagggggcactgccaagacaataagctaggctactgggtccagctactactttggtgggattcaggtgagtctccatgcacttcacatgttacccagtgttcttgttacttccaaggagaaccaagaatggctctgtcacactcgaagccaggcttgatcaataaacacaatggtattccacgtcaaaaaaaaaaaaaaaaaa

SEQ ID NO: 4 - DNA, 2800 bp, Homo sapiens
gtcccagctactctggaggctgtggcaggagaactgcttgaacctgggaagtggaggttgcagtgagctgatattgtgccactgcactgcagcctgggcgacagagtgagactgtcttaaaaacaaacaaacaaaaagattaaaggagagagcggctgcattttgggaggccaaggcgggctgattgcctgagctcaggagttggagaccagcctgggcaacatggtgaaacccggtctctactaaaatatgtttttaaaaaaaattagctgggcatgcacctgtagtcccagctactcgggaggctgaggcaggagaattgcttgaaccggagaggtggaggttgcagtgagctgagatcatgccaatgcactccagcgtgggtgacagagcaagactccatctcaaaaaaaaggagagagcacagtgtctggtatatcctaagtgctccacgagtgccagacattggttatacttcatggaggtgaggtacacagaggtgtctaggtcatgcggactcactccatcctccagttaaccatgactccctccccacccacctcctgagttctctctgctgtcaaactttcctctcatatctccagtttcccacccaaaagtccaaacactcaagattccttctcatctccaacccataaaatctgatcggcttctccacactcaagtgtaaatgactgatggccatttgattgacactggaggggctggcacatttttagcacatttttagccaaaggtatgatgtgctgatagcggactctcaggccacaagccagtctgaggacagggaaagtttgaggaggtcactcttccagaaccctttggtgataacctttctggcaggcctggtacccccagagtgaaaagggctccagggtcaagacccagctctgcccctcattgctgtgtgaccttcagcaactcacttgatttatctggacctcagcttcttcatctgtctaatgggataataatctttgtcctgccctcctcacagggctgttcagaattaaatgcatctgaaaatgatctgcatttgtgtcttggactgtggttatttgttcaagtgtctgtctaccctgcctgcacagggagcgggtcagggtcttccctgggacttcatgcacaggtctttcctgggacttcatgtaggaccagccatcccgtcagtgctcagtgaacatgagctgcttccctgtgggatgtctgggaggtgagtggaaggccttcctagcaggcacatactggaacataatcaatccctcccatgcatacagctcacacccacattcttatccattcccaccatgcccctcagtggcctggggagactaggaataccccacttgacagataaggaagctgaggccaggggaggtcaaggcatttgtctgaggttgcacaaccaggagtggtgaagctgggagtcacacacaaggaatcactgcaaagtctgtgtccttcccactctgagtaggacttgaaccagggatgtccgatgctgatgcttgtgtcctgaccactaccttgcagcgtcctcacccacacccaccccaggcctgggaggcaggggagcaccagcggtctgggagtgaaagttgcttccctgtgggatgtctgggaggtgggtagaaggccttcctagcaggcacagctgcccaccggatgagtatgtccagggggagaggagccccctgtcccaggatgtgggcaaaaacctggagaagctgaagctggactttgaggatggcccctgactccatcctgggacagccacctccccttccttctgccccctccagtccctcctctccttccctgctctccctcctacctctttccatctccccctttctgcctgtgattcctcctgtgactggggcaagatccttcacctctgtcctccctgagcctcagtggctcacctggaacatagggtgatgcctgcagaagtattttagggttactttatgagataaattggtagactgtttttcccgggtcagccagccaggggttcggtgaataggagcgaacgctgctgccattcccttctcccctagttcatcctggaggctctccgctctccccctggttctaagtcccctcctcctgcaccgtatcccccctcccatccaacccagtccccactgaggcactgagacagggtcttggcccagggtccccacccacctgagctccgcatgtgagccgaccttcacccgcctgcctcagtttcccgtccacgaaaggggtgagcgcccccacgtctcctgggactgtccttgggtccgatgcggggctgtggggaggtgccagggtgtgggcgcagcagggagcgggtgtgttaggcccccgcccatcccgcgcccgagccccatctggctcgggctggcacctcgaatccacgtgatttctcggcagcagccgccagttccatgcactggcggagcagctctaggcggcggcttctactttcagtttcgtgcagagcgcggaggagccgcgagcgctgagggtgagtgccgggagctctgagggtgagtgccgggcgtgccgcggggctgcgggacccgggctggggcgagcggaagggagaggatcggggttcgaattctgcacggagaggggtggaggggatgtcagaggccctggagcgggaggtgcgggtggccgggtggctggccgatggatagctgggtaagcggacagggcgccagtggcccggcagcgcgcacagcatgcgccccggcagtttggaggaagagttgccggcccg

SEQ ID NO: 5 - DNA, 3501 bp, Homo sapiens
actcacttgctggtaacctggaatatgctcctttggtggttgtgcctgataaaagtcaacaatacgtttacaattttttatcctcaattcagaacttggtattttcattaagtcacccagaagcgcaactgaactagcagtatctccagcataagttatttcatccgatactttctttttcaaaaatggcaagccacactattttatgatatcatatgcagattctacaaaatgcggctggttttcaatttttactgcacacagattcagaatttcaaatgtctgtactaaatcccttaagggaagttcattctgataacactgtaccagtttcttgacagatttaagttgcttttcttctaagccttctttagcagtctcttcaaaaagtttgatgacaccattaaggtccacaggcttcaaaacaccagttccacatggcgagggaggccttgggaaacatcatcatgatggaaggcaaaggggaagtaaagaccttcttcatggtggcaggaaagagacagcatattgctatgtattttaacaccacttatttccccccttttttctttcaatatcatggattaaatagtgtattttaaatgagtgtgaggctcacttggactctattccatccctttggccaatttttttccatttttaatccaagaatcttaaagtttgaatgatggtcaccttgttatatattttattatgtttggaagcttttttgctattgtctgatcctcccaacaaatagccttttgatagaattgcctttattcgtttctttatatctaattctaacttaagattagacttaaaagtaacttcagcggttttattatgaaatatttaaggcatataaatactgcctcttttctctaaatgactgctaatataattttaccaagttctgttaaagtatcctgttgtacataaagtgctgttgttttttatctgcctgttaacttagagtgggttttattttctttcactttctgtggtcgaaggcaagtatgtcttagtcagtttgggctgttataatgaaacactgtagactgggtggcttaaacaacagaaacttgtttctcacgttctgaaagccgggaagtccccaatcagggagccagcaggcctggtgtctggagaggatgcgcttcctggtttgcagacggcaccttctcattgtgtcctcacagggcagagagcaaaaagaaagggcaagcgctctccagcctcttcctttttcctttttttttttgtttgtttgagatggagtcttgctctgttgcccagactggagtgcggtggcatgatcttggctcaatgcaacctctgcctcctggcttcaagtgattctcctgcctcagcctcctgagtagctgggactacaggtgcctgccaccatgcctgactaatttttatgtttttattagagatggggtttcatcatgttggccagactggttttagactcctgacctcaagtgatctacctaccttggcctcccaaagtgctgggattacaggcgtgaaccaccgcacccggcctccagcctcttcttgtgagggcagtaatcccatcataaggggtccaccctcttgacctaatcacttcgcaaaggtcccacttccaaataccatcacactgggtatttaggcttcaccaggtgaattggtgggggaggtgggacacaaacattcagtccacggcaagtttttatctcaatttgctaaagcctctctttatttctcctgttacagtgtgataatgttttttaaaatatcatttagctggctgtggtgacccatgcctgcagtcccagctactcaggaggctaaggcaaaaggatcacatgaacctaggagtttgaatccagcctgggcaacatagaaagaccctgtctcttaaaaaaataaataaataaataaataaataaataaataaataaataaataaaggaaagaggctgggcacagtggctcatgcctataatcccagcactttgggaggccgaggcgggcagatcacctgaagtcgggagttcaagaccagcctgaccaacatggagaaaccccttctctactaaaaatacaaaattagccgggtgtagtggcacatgcctgtaatcccactacttgggaggctgaggcaagagaattgcttgaaccagaaggtggaggttgtggtgagccgagatcatgccattgcactccagcctgggcaacaagaatgaaactccatctcaaaaaaaaaaaaaaaaaaaaaaagaaagaaaagaaaagaagcacttaaagaggctgaggcaggcagatcacttgaagtcaggagttcaagaccagcctggccaacatggtgaaacctagtctctactaaaaatactagaattagccagacgtggtggcacatgcctgtaatcccagctactcgggaggctgaggcatgagacttgcttgaacctaggaggtggaggttgcagtgagctgagaacatgccattgcactccagcctgggcaacagagcaagactccatctcaagaaaaaagaaagagagagaaagagaaatatagacatatgttattagtcccccaggacctcttcaaaagatctctcttttcctttcttatttctaattaccatactacataccacctccattatgtcagaatgtgccctatttacgtggatttgtctaaagtcatggaactctgagttacattccccgtatttcacagagacacaatcagagcacacgctgatcttccgcctctcttcatttcctccatgtactctgtagctacctgttctcttctttgttcgtaggaagatctctgttgcgtggtgtgggagtttgtgccagctcatctttttattgtgctttctgtgagcagctatcaatgcctccaggtcttctctttaagaagaatgtttactctcaattgtttaaaaaactaactaaataggaagaagattgcaaatcctaacctttttggggttttatattgggggcaaaagttggcatctgcagtaaggattatttcattttcatctgaataaatgagcagtaataactttgctgacttgcagggctcattgtgatgccaaaggacattgtaaggtgcatatctgaattggaggaggaagaggggcatcctctgagccaggtgtggcaggaggtgacatcaaggagggttaggctgcgtaaacccagggacacaaggagctgcagtcggggaagaacaaggtctagcggatggagttggggggactgtgtctggagccaatgttctcagatgtgtctccggcaggcaggggcaagggctggtgcctggtttacagcattccttttggagtctggaatagtggggaaaactccctgtcttctctccccttaggagtctgcactatggaaacaacctgtcaatccagctcaaggcacacatagcccagacacccatgagaccctctccgtggggaccctagagcacctatcatga

SEQ ID NO: 6 - DNA, 50 bp, Homo sapiens
cggagctcaggtgggtggggaccctgggccaagaccctgtctcagtgcct

SEQ ID NO: 7 - DNA, 20 bp, Artificial Sequence
  Forward Primer Sequence
tccaatcccgcgccgggcca

SEQ ID NO: 8 - DNA, 23 bp, Artificial Sequence
  Reverse Primer Sequence
ctgtggcccggcgcgggattgga

SEQ ID NO: 9 - DNA, 31 bp, Artificial Sequence
  Forward Primer Sequence
gttcctttttgaattctgccagctcaacttg

SEQ ID NO: 10 - DNA, 33 bp, Artificial Sequence
  Reverse Primer Sequence
gttgagctggcagaattcaaaaaggaacagggc

SEQ ID NO: 11 - DNA, 19 bp, Artificial Sequence
  Forward Primer Sequence
gccatcgtgggagggggcc

SEQ ID NO: 12 - DNA, 20 bp, Artificial Sequence
  Reverse Primer Sequence
cccctcccacgatggctggg

SEQ ID NO: 13 - DNA, 26 bp, Artificial Sequence
  Forward Primer Sequence
gctgggtacagtggaccaaagctaga

SEQ ID NO: 14 - DNA, 25 bp, Artificial Sequence
  Reverse Primer Sequence
gctttggtccactgtacccagcggg

SEQ ID NO: 15 - DNA, 28 bp, Artificial Sequence
  Forward Primer Sequence
gcctgctgactttcttctgcgtctgcac

SEQ ID NO: 16 - DNA, 28 bp, Artificial Sequence
  Reverse Primer Sequence
cagacgcagaagaaagtcagcaggctgt

SEQ ID NO: 17 - DNA, 23 bp, Artificial Sequence
  Forward Primer Sequence
gcacctgccaccccttccttagc

SEQ ID NO: 18 - DNA, 22 bp, Artificial Sequence
  Reverse Primer Sequence
ggaaggggtggcaggtgcagga

SEQ ID NO: 19 - DNA, 29 bp, Artificial Sequence
  Forward Primer Sequence
cacctggtgaaagatttgcctctctgtcc

SEQ ID NO: 20 - DNA, 28 bp, Artificial Sequence
  Reverse Primer Sequence
cagagaggcaaatctttcaccaggtggc

SEQ ID NO: 21 - DNA, 28 bp, Artificial Sequence
  Forward Primer Sequence
gaaccagctggaaaatgaaggctgtcgg

SEQ ID NO: 22 - DNA, 28 bp, Artificial Sequence
  Reverse Primer Sequence
gacagccttcattttccagctggttccc

SEQ ID NO: 23 - DNA, 26 bp, Artificial Sequence
  Forward Primer Sequence
gctgccacctcagtcacctccacctc

SEQ ID NO: 24 - DNA, 26 bp, Artificial Sequence
  Reverse Primer Sequence
gtggaggtgactgaggtggcagcttc

SEQ ID NO: 25 - DNA, 24 bp, Artificial Sequence
  Forward Primer Sequence
gcagctgagccagatgggactgtc

SEQ ID NO: 26 - DNA, 23 bp, Artificial Sequence
  Reverse Primer Sequence
gtcccatctggctcagctgcagc

SEQ ID NO: 27 - DNA, 21 bp, Artificial Sequence
  Forward Primer Sequence
gagggcctcacccacctggca

SEQ ID NO: 28 - DNA, 21 bp, Artificial Sequence
  Reverse Primer Sequence
ccaggtgggtgaggccctcgg

SEQ ID NO: 29 - DNA, 25 bp, Artificial Sequence
  Forward Primer Sequence
cccaggccctggatggatcccccat

SEQ ID NO: 30 - DNA, 25 bp, Artificial Sequence
  Reverse Primer Sequence
caaatgggggatccatccagggcct

SEQ ID NO: 31 - DNA, 32 bp, Artificial Sequence
  Forward Primer Sequence
gcatccaagtcatctgcctctggaataacccc

SEQ ID NO: 32 - DNA, 33 bp, Artificial Sequence
  Reverse Primer Sequence
ggttattccagaggcagatgacttggatgctag

SEQ ID NO: 33 - DNA, 24 bp, Artificial Sequence
  Forward Primer Sequence
cctgaagagctaggagcccaggct

SEQ ID NO: 34 - DNA, 24 bp, Artificial Sequence
  Reverse Primer Sequence
ctgggctcctagctcttcaggtgc